(19) United States
(12) Patent Application Publication
(10) Pub. No.: US 2008/0275018 A1
(43) Pub. Date: Nov. 6, 2008

(54) ANTIBACTERIAL AMIDE-MACROCYCLES V

(75) Inventors: Rainer ENDERMANN, Wuppertal (DE); Kerstin EHLERT, Velbert (DE); Siegfried RADDATZ, Koeln (DE); Martin MICHELS, Koeln (DE); Yolanda CANCHO-GRANDE, Leverkusen (DE); Stefan WEIGAND, Penzberg (DE); Karin FISCHER, Solingen (DE)

Correspondence Address: MORRISON & FOERSTER LLP, 12531 HIGH BLUFF DRIVE, SUITE 100, SAN DIEGO, CA, 92130-2040, US

(73) Assignee: AiCuris GmbH & Co. KG, Wuppertal (DE)

(21) Appl. No.: 11/906,088

(22) Filed: Sep. 28, 2007

Related U.S. Application Data
(63) Continuation of application No. PCT/EP06/02617, filed on Mar. 22, 2006.

(30) Foreign Application Priority Data
Mar. 30, 2005 (DE) 10 2005 014 245.1

(51) Int. Cl. A61K 31/395; C07D 225/08; A61P 31/04

(52) U.S. Cl. 514/183; 540/461

(57) ABSTRACT

The invention relates to antibacterial amide-macrocycles of formula (I), in which R²⁶ represents hydrogen, halogen, amino or methyl, R⁷ represents a group of formula (II), (III), (IV) or (V), whereby R¹ represents hydrogen or hydroxy and * is the linkage site to the carbon atom, R² represents hydrogen or methyl and methods for their production, their use for the treatment and/or prophylaxis of diseases as well as their use for the production of medicaments for the treatment and/or prophylaxis of diseases, in particular of bacterial infections.

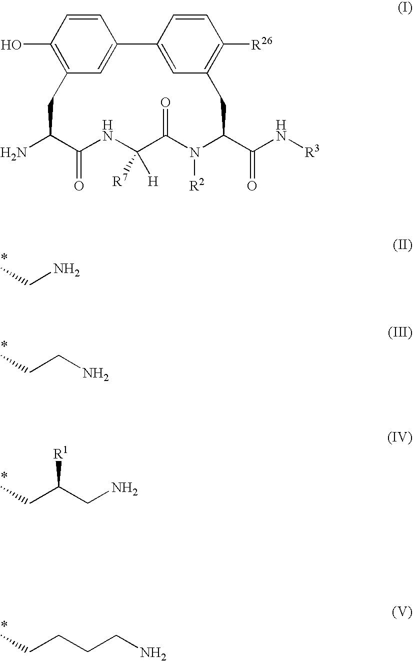

### CROSS REFERENCE TO RELATED APPLICATIONS

This application is a continuation of pending international application PCT/EP2006/002617, filed Mar. 22, 2006, designating US, which claims priority from German patent application DE 10 2005 014 245.1, filed Mar. 30, 2005. The contents of the above-referenced applications are incorporated herein by this reference in their entirety.

### BACKGROUND OF THE INVENTION

The invention relates to antibacterial amide macrocycles and methods for their preparation, their use for the treatment and/or prophylaxis of diseases, as well their use for the production of medicaments for the treatment and/or prophylaxis of diseases, in particular of bacterial infections.

WO 03/106480 and WO 04/012816 describe macrocycles of the biphenomycin B type which have antibacterial activity and have amide and ester substituents respectively.

U.S. Pat. No. 3,452,136, thesis of R. U. Meyer, Stuttgart University, Germany 1991, thesis of V. Leitenberger, Stuttgart University, Germany 1991, Synthesis (1992), (10), 1025-30, J. Chem. Soc., Perkin Trans. 1 (1992), (1), 123-30, J. Chem. Soc., Chem. Commun. (1991), (10), 744, Synthesis (1991), (5), 409-13, J. Chem. Soc., Chem. Commun. (1991), (5), 275-7, J. Antibiot. (1985), 38(11), 1462-8, J. Antibiot. (1985), 38(11), 1453-61 describe the natural product biphenomycin B as having antibacterial activity. Some steps in the synthesis of biphenomycin B are described in Synlett (2003), 4, 522-526.

Chirality (1995), 7(4), 181-92, J. Antibiot. (1991), 44(6), 674-7, J. Am. Chem. Soc. (1989), 111(19), 7323-7, J. Am. Chem. Soc. (1989), 111(19), 7328-33, J. Org. Chem. (1987), 52(24), 5435-7, Anal. Biochem. (1987), 165(1), 108-13, J. Org. Chem. (1985), 50(8), 1341-2, J. Antibiot. (1993), 46(3), C-2, J. Antibiot. (1993), 46(1), 135-40, Synthesis (1992), (12), 1248-54, Appl. Environ. Microbiol. (1992), 58(12), 3879-8, J. Chem. Soc., Chem. Commun. (1992), (13), 951-3 describe a structurally related natural product, biphenomycin A, which has a further substitution with a hydroxy group on the macrocycle.

The natural products in terms of their properties do not comply with the requirements for antibacterial medicaments. Although structurally different agents with antibacterial activity are available on the market, the development of resistance is a regular possibility. Novel agents for a good and more effective therapy are therefore desirable.

### SUMMARY OF THE INVENTION

One object of the present invention is therefore to provide novel and alternative compounds with the same or improved antibacterial activity for the treatment of bacterial diseases in humans and animals.

It has surprisingly been found that certain derivatives of these natural products in which the carboxy group of the natural product is replaced by an amide group which comprises a basic group have antibacterial activity against biphenomycin-resistant S. aureus Strains (RN4220Bi^{R} and T17).

In addition, the derivatives show an improved spontaneous resistance rate for S. aureus wild-type strains and biphenomycin-resistant S. aureus Strains.

The invention relates to compounds of formula

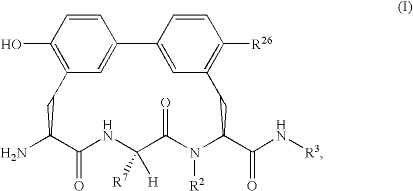

in which
R²⁶ represents hydrogen, halogen, amino or methyl,
R⁷ represents a group of formula

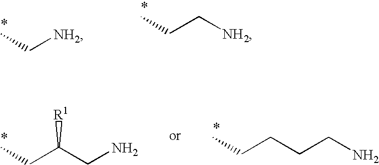

whereby
R¹ represents hydrogen or hydroxy,
* is the linkage site to the carbon atom,
   R² represents hydrogen or methyl,
   R³ represents a group of formula

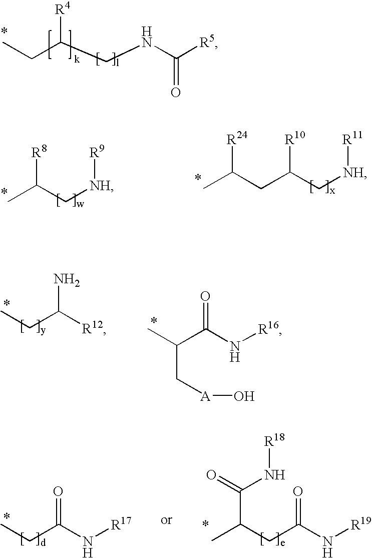

whereby
* is the linkage site to the nitrogen atom,
A represents a bond or phenyl,
R⁴ represents hydrogen, amino or hydroxy,
R⁵ represents a group of formula

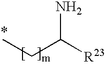

wherein
* is the linkage site to the carbon atom,
R²³ represents hydrogen or a group of formula *—(CH₂)ₙ—OH or *—(CH₂)ₒ—NH₂,
   wherein
   * is the linkage site to the carbon atom,
   n and o independently of one another are a number 1, 2, 3 or 4,
m is a number 0 or 1,
R⁸ and R¹² independently of one another represent a group of formula *—CONHR¹⁴ or *—CH₂CONHR¹⁵,
   wherein
   * is the linkage site to the carbon atom,
   R¹⁴ and R¹⁵ independently of one another represent a group of formula

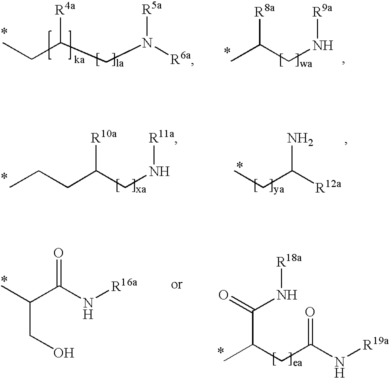

wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
   wherein
   * is the linkage site to the carbon atom,
   Z1a and Z2a independently of one another are a number 1, 2 or 3,
   R^{13a} represents hydrogen or methyl,
   and
   R^{14a} and R^{15a} independently of one another represent a group of formula

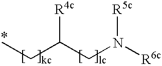

wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula

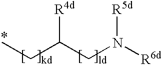

wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1, and
ld is a number 1, 2, 3 or 4,
R^{18a} and R^{19a} independently of one another represent hydrogen or a group of formula

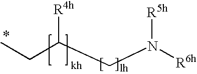

wherein
* is the linkage site to the nitrogen atom,
R^{4h} represents hydrogen, amino or hydroxy,
R^{5h} represents hydrogen, methyl or aminoethyl,
R^{6h} represents hydrogen or aminoethyl,
or
R^{5h} and R^{6h} together with the nitrogen atom to which they are bonded form a piperazine ring,
kh is a number 0 or 1,
and
lh is a number 1, 2, 3 or 4,
whereby R^{8a} and R^{19a} are not simultaneously hydrogen,
ka is a number 0 or 1,
ea is a number 1, 2 or 3,
and
la, wa, xa and ya independently of one another are a number 1, 2, 3 or 4,
R⁹ and R¹¹ independently of one another represent hydrogen, methyl, *—C(NH₂)═NH or a group of formula

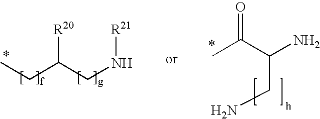

wherein
* is the linkage site to the nitrogen atom,
R²⁰ represents hydrogen or *—(CH₂)ᵢ—NHR²²,
   wherein
   R²² represents hydrogen or methyl,
   and
   i is a number 1, 2 or 3,
R²¹ represents hydrogen or methyl,
f is a number 0, 1, 2 or 3,
g is a number 1, 2 or 3,
and
h is a number 1, 2, 3 or 4,
or
R⁸ represents *—(CH₂)_{Z1}—OH,
   wherein
   * is the linkage site to the carbon atom,
   Z1 is a number 1, 2 or 3,
and
R⁹ represents a group of formula

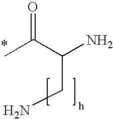

wherein
* is the linkage site to the nitrogen atom,
and
h is a number 1, 2, 3 or 4,
R¹⁰ represents amino or hydroxy,
R¹⁶ and R¹⁷ independently of one another represent a group of formula

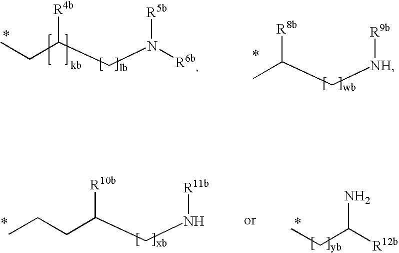

wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *—(CH₂)_{Z1b}—OH, *—(CH₂)_{Z2b}—NHR^{13b}, *—CONHR^{14b} or *—CH₂CONHR^{15b},
   wherein
   * is the linkage site to the carbon atom,
   R^{13b} represents hydrogen or methyl,
   and
   Z1b and Z2b independently of one another are a number 1, 2 or 3,
   and
   R^{14b} and R^{15b} independently of one another represent a group of formula

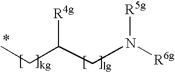

wherein
* is the linkage site to the nitrogen atom,
R^{4g} represents hydrogen, amino or hydroxy,
R^{5g} represents hydrogen, methyl or aminoethyl,
R^{6g} represents hydrogen or aminoethyl,
kg is a number 0 or 1, and
lg is a number 1, 2, 3 or 4,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
R¹⁸ and R¹⁹ independently of one another represent hydrogen or a group of formula

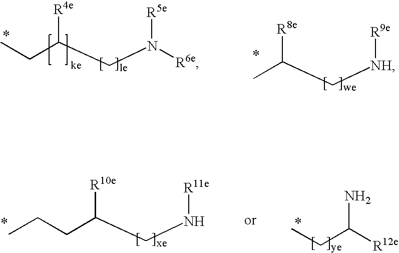

wherein
* is the linkage site to the nitrogen atom,
R^{4e} represents hydrogen, amino or hydroxy,
R^{5e} represents hydrogen, methyl or aminoethyl,
R^{6e} represents hydrogen or aminoethyl,
or
R^{5e} and R^{6e} together with the nitrogen atom to which they are bonded form a piperazine ring,
R^{8e} and R^{12e}, independently of one another represent *—(CH₂)_{Z1e}—OH or *—(CH₂)_{Z2e}—NHR^{13e},
   wherein
   * is the linkage site to the carbon atom,
   R^{13e} represents hydrogen or methyl,
   and
   Z1e and Z2e independently of one another are a number 1, 2 or 3,
R^{9e} and R^{11e} independently of one another represent hydrogen or methyl,
R^{10e} represents amino or hydroxy,
ke is a number 0 or 1,
and
le, we, xe and ye independently of one another are a number 1, 2, 3 or 4,
whereby R¹⁸ and R¹⁹ are not simultaneously hydrogen,
R²⁴ represents a group of formula *—CONHR
   wherein
   * is the linkage site to the carbon atom,
   R²⁵ represents a group of formula

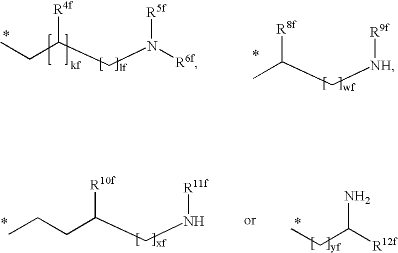

wherein
the linkage site to the nitrogen atom,
R^{4f} represents hydrogen, amino or hydroxy,
R^{5f} represents hydrogen, methyl or aminoethyl,
R^{6f} represents hydrogen or aminoethyl,
or
R^{5f} and R^{6f} together with the nitrogen atom to which they are bonded form a piperazine ring,
R^{8f} and R^{12f} independently of one another represent *—(CH₂)_{Z1f}—OH or *—(CH₂)_{Z2f}—NHR^{13f},
   wherein
   * is the linkage site to the carbon atom, R^{13f} represents hydrogen or methyl,
   and
   Z1f and Z2f independently of one another are a number 1, 2 or 3,
R^{9f} and R^{11f} independently of one another represent hydrogen or methyl,
R^{10f} represents amino or hydroxy,
kf is a number 0 or 1,
and
lf, wf, xf and yf independently of one another are a number 1, 2, 3 or 4,
d and e independently of one another are a number 1, 2 or 3,
k is a number 0 or 1,
l, w, x and y independently of one another are a number 1, 2, 3 or 4,

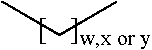

independently of one another may when w, x or y equals 3 carry a hydroxy group,

and their salts, their solvates and the solvates of their salts.

Compounds of the invention are the compounds of formula (I) and the salts, solvates and solvates of the salts thereof, as well as the compounds which are encompassed by formula (I) and are mentioned hereinafter as exemplary embodiment(s), and the salts, solvates and solvates of the salts thereof, insofar as the compounds which are encompassed by formula (I) and are mentioned hereinafter are not already salts, solvates and solvates of the salts.

The compounds of the invention may, depending on their structure, exist in stereoisomeric forms (enantiomers, diastereomers). The invention therefore relates to the enantiomers or diastereomers and their respective mixtures. The stereoisomerically pure constituents can be isolated from such mixtures of enantiomers and/or diastereomers in a known way by known processes such as chromatography on a chiral phase or crystallization using chiral amines or chiral acids.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates for the purposes of the invention refer to those forms of the compounds which form a complex in the solid or liquid state through coordination with solvent molecules. Hydrates are a special form of solvates in which coordination takes place with water.

Halogen stands for fluorine, chlorine, bromine and iodine.

A symbol # on a carbon atom means that the compound is in enantiopure form with respect to the configuration at this carbon atom, meaning in the context of the present invention an enantiomeric excess of more than 90% (>90% ee).

In the formulae of the groups which R³ can represent, the end point of the line beside which there is in each case an * does not represent a carbon atom or a CH₂ group but forms part of the bond to the nitrogen atom to which R³ is bonded.

In the formulae of the groups which R⁷ can represent, the end point of the line beside which there is in each case an * does not represent a carbon atom or a CH₂ group but forms part of the bond to the carbon atom to which R⁷ is bonded.

Preference is given in the context of the present invention to compounds of formula (I) in which
R²⁶ represents hydrogen, halogen, amino or methyl,
R⁷ represents a group of formula

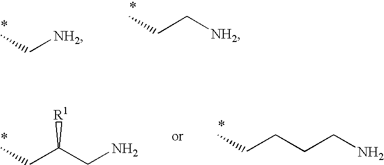

whereby
R¹ represents hydrogen or hydroxy,
* is the linkage site to the carbon atom,
   R² represents hydrogen or methyl,
   R³ represents a group of formula

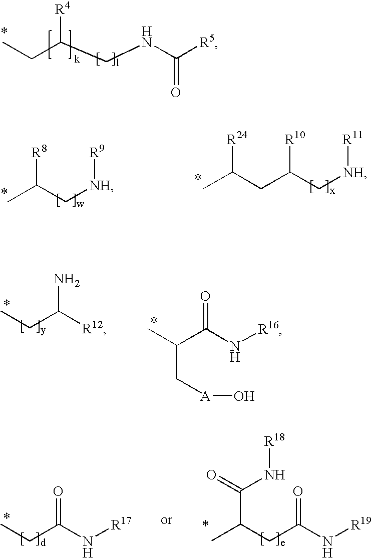

whereby
* is the linkage site to the nitrogen atom,
A represents a bond or phenyl,
R⁴ represents hydrogen, amino or hydroxy,
R⁵ represents a group of formula

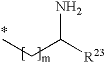

wherein
* is the linkage site to the carbon atom,
R²³ represents hydrogen or a group of formula *—(CH₂)ₙ—OH or *—(CH₂)ₒ—NH₂,
   wherein
   * is the linkage site to the carbon atom,
   n and o independently of one another are a number 1, 2, 3 or 4,
m is a number 0 or 1,
R⁸ and R¹² independently of one another represent a group of formula *—CONHR¹⁴ or *—CH₂CONHR¹⁵,
   wherein
   * is the linkage site to the carbon atom,
   R¹⁴ and R¹⁵ independently of one another represent a group of formula

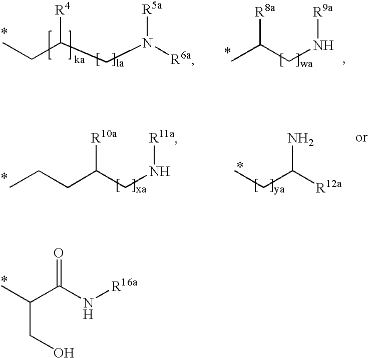

wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
   wherein
   * is the linkage site to the carbon atom,
   Z1a and Z2a independently of one another are a number 1, 2 or 3,
   R^{13a} represents hydrogen or methyl,
   and
   R^{14a} and R^{15a} independently of one another represent a group of formula

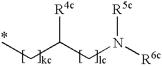

wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula

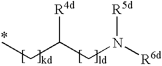

wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
R⁹ and R¹¹ independently of one another represent hydrogen, methyl, *—C(NH₂)═NH or a group of formula

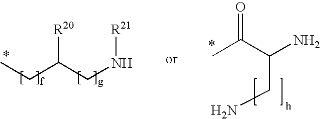

wherein
* is the linkage site to the nitrogen atom,
R²⁰ represents hydrogen or *—(CH₂)ᵢ—NHR²²,
   wherein
   R²² represents hydrogen or methyl,
   and
   i is a number 1, 2 or 3,
R²¹ represents hydrogen or methyl,
f is a number 0, 1, 2 or 3,
g is a number 1, 2 or 3, and
h is a number 1, 2, 3 or 4,
or
R⁸ represents *—(CH₂)_{Z1}—OH,
   wherein
   * is the linkage site to the carbon atom,
   Z1 is a number 1, 2 or 3,
and
R⁹ represents a group of formula

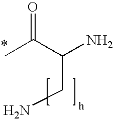

wherein
* is the linkage site to the nitrogen atom,
and
h is a number 1, 2, 3 or 4,
R¹⁰ represents amino or hydroxy,
R¹⁶ and R¹⁷ independently of one another represent a group of formula

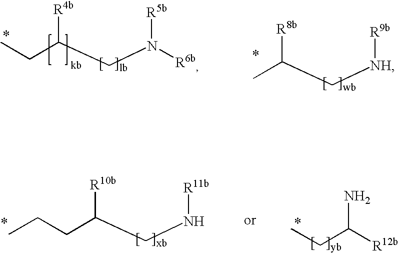

wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *—(CH₂)_{Z1b}—OH, *—(CH₂)_{Z2b}—NHR^{13b}, *—CONHR^{14b} or *—CH₂CONHR^{15b},
wherein
* is the linkage site to the carbon atom,
R^{13b} represents hydrogen or methyl,
and
Z1b and Z2b independently of one another are a number 1, 2 or 3, and
R^{14b} and R^{15b} independently of one another represent a group of formula

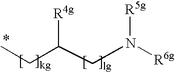

wherein
* is the linkage site to the nitrogen atom,
R^{4g} represents hydrogen, amino or hydroxy,
R^{5g} represents hydrogen, methyl or aminoethyl,
R^{6g} represents hydrogen or aminoethyl,
kg is a number 0 or 1 and
lg is a number 1, 2, 3 or 4,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
R¹⁸ and R¹⁹ independently of one another represent hydrogen or a group of formula

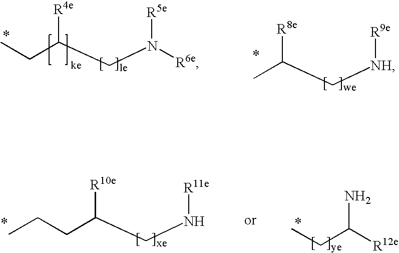

wherein
* is the linkage site to the nitrogen atom,
R^{4e} represents hydrogen, amino or hydroxy,
R^{5e} represents hydrogen, methyl or aminoethyl,
R^{6e} represents hydrogen or aminoethyl,
or
R^{5e} and R^{6e} together with the nitrogen atom to which they are bonded form a piperazine ring,
R^{8e} and R^{12e} independently of one another represent *—(CH₂)_{Z1e}—OH or *—(CH₂)_{Z2e}—NHR^{13e},
   wherein
   * is the linkage site to the carbon atom,
   R^{3e} represents hydrogen or methyl,
   and
   Z1e and Z2e independently of one another are a number 1, 2 or 3,
R^{9e} and R^{11e} independently of one another represent hydrogen or methyl,
R^{10e} represents amino or hydroxy,
ke is a number 0 or 1,
and
le, we, xe and ye independently of one another are a number 1, 2, 3 or 4,
whereby R¹⁸ and R¹⁹ are not simultaneously hydrogen,
R²⁴ represents a group of formula *—CONHR²⁵,
   wherein
   * is the linkage site to the carbon atom,
   R²⁵ represents a group of formula

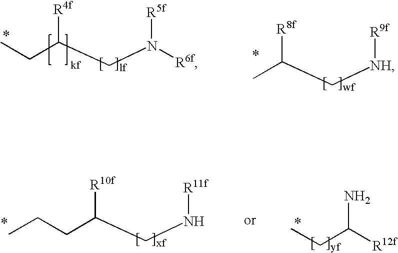

wherein
* is the linkage site to the nitrogen atom,
R^{4f} represents hydrogen, amino or hydroxy,
R^{5f} represents hydrogen, methyl or aminoethyl,
R^{6f} represents hydrogen or aminoethyl,
or
R^{5f} and R^{6f} together with the nitrogen atom to which they are bonded form a piperazine ring,
R^{8f} and R^{12f} independently of one another represent *—(CH₂)_{Z1f}—OH or *—(CH₂)_{Z2f}—NHR^{3f},
   wherein
   * is the linkage site to the carbon atom,
   R^{13f} represents hydrogen or methyl,
   and
   Z1f and Z2f independently of one another are a number 1, 2 or 3,
R^{9f} and R^{11f} independently of one another represent hydrogen or methyl,
R^{10f} represents amino or hydroxy,
kf is a number 0 or 1,
and
lf, wf, xf and yf independently of one another are a number 1, 2, 3 or 4,
d and e independently of one another are a number 1, 2 or 3,
k is a number 0 or 1,
l, w, x and y independently of one another are a number 1, 2, 3 or 4,

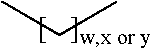

independently of one another may when w, x or y equals 3 carry a hydroxy group,
and their salts, their solvates and the solvates of their salts.

Preference is also given in the context of the present invention to compounds of formula

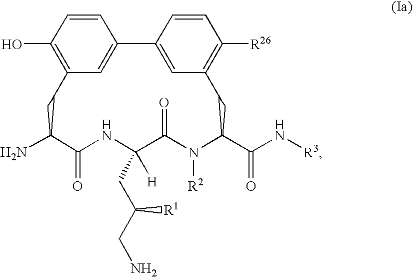

in which
R²⁶ represents hydrogen, halogen, amino or methyl,
R¹ represents hydrogen or hydroxy,
R² represents hydrogen or methyl,
R³ is as defined above,
and their salts, their solvates and the solvates of their salts.

Preference is also given in the context of the present invention to compounds of formula (I) or (Ia) in which
R²⁶ represents hydrogen, chlorine or methyl.

Preference is also given in the context of the present invention to compounds of formula (I) or (Ia) in which
R²⁶ represents hydrogen.

Preference is also given in the context of the present invention to compounds of formula (I) or (Ia) in which
R³ represents a group of formula

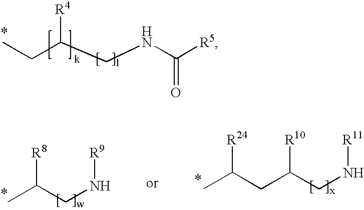

whereby
* is the linkage site to the nitrogen atom,
R⁴ represents hydrogen, amino or hydroxy,
R⁵ represents a group of formula

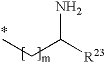

wherein
* is the linkage site to the carbon atom,
R²³ represents hydrogen or a group of formula *—(CH₂)ₙ—OH or *—(CH₂)ₒ—NH₂,
   wherein
   * is the linkage site to the carbon atom,
   n and o independently of one another are a number 1, 2, 3 or 4,
m is a number 0 or 1,
R⁸ represents a group of formula *—CONHR¹⁴ or *—CH₂CONHR¹⁵,
   wherein
   * is the linkage site to the carbon atom,
   R¹⁴ and R¹⁵ independently of one another represent a group of formula

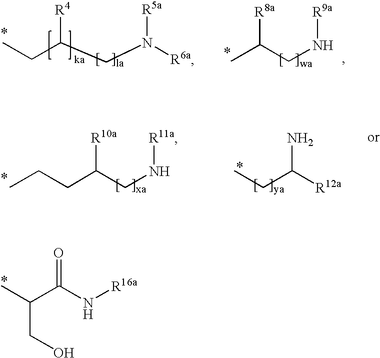

wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
   wherein
   * is the linkage site to the carbon atom,
   Z1a and Z2a independently of one another are a number 1, 2 or 3,
   R^{13a} represents hydrogen or methyl,
   and
   R^{14a} and R^{15a} independently of one another represent a group of formula

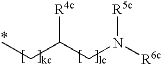

wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula

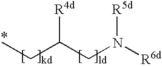

wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
R⁹ and R¹¹ independently of one another represent hydrogen, methyl, *—C(NH₂)═NH or a group of formula

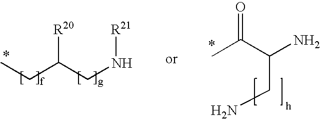

wherein
* is the linkage site to the nitrogen atom,
R²⁰ represents hydrogen or *—(CH₂)ᵢ—NHR²²,
   wherein
   R²² represents hydrogen or methyl, and
   i is a number 1, 2 or 3,
   R²¹ represents hydrogen or methyl,
f is a number 0, 1, 2 or 3,
g is a number 1, 2 or 3, and
h is a number 1, 2, 3 or 4,
or
R⁸ represents *—(CH₂)_{Z1}—OH
   wherein
   * is the linkage site to the carbon atom,
   Z1 is a number 1, 2 or 3,
and
R⁹ represents a group of formula

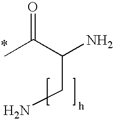

wherein
* is the linkage site to the nitrogen atom,
and
h is a number 1, 2, 3 or 4,
R¹⁰ represents amino or hydroxy,
R²⁴ represents a group of formula *—CONHR²⁵,
   wherein
   * is the linkage site to the carbon atom,
   R²⁵ represents a group of formula

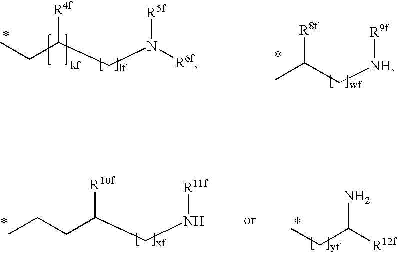

wherein
* is the linkage site to the nitrogen atom,
R^{4f} represents hydrogen, amino or hydroxy,
R^{5f} represents hydrogen, methyl or aminoethyl,
R^{6f} represents hydrogen or aminoethyl,
or
R^{5r} and R^{6f} together with the nitrogen atom to which they are bonded form a piperazine ring,
R^{8f} and R^{12f} independently of one another represent *—(CH₂)_{Z1f}—OH or *—(CH₂)_{Z2f}—NHR^{13f},
   wherein
   * is the linkage site to the carbon atom,
   R^{13f} represents hydrogen or methyl,
   and
   Z1f and Z2f independently of one another are a number 1, 2 or 3,
R^{9f} and R^{11f} independently of one another represent hydrogen or methyl,
R^{10f} represents amino or hydroxy,
kf is a number 0 or 1, and
lf, wf, xf and yf independently of one another are a number 1, 2, 3 or 4,
k is a number 0 or 1,
l, w and x independently of one another are a number 1, 2, 3 or 4,

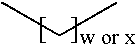

independently of one another may when w or x equals 3 carry a hydroxy group,
and their salts, their solvates and the solvates of their salts.

Particular preference is given in the context of the present invention to compounds of formula (I) or (Ia) in which
R³ represents a group of formula

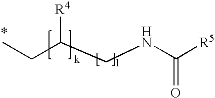

whereby
* is the linkage site to the nitrogen atom,
R⁴ represents hydrogen, amino or hydroxy,
R⁵ represents a group of formula

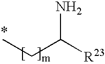

wherein
* is the linkage site to the carbon atom,
   R²³ represents hydrogen or a group of formula *—(CH₂)ₙ—OH or *—(CH₂), NH₂,
      wherein
      * is the linkage site to the carbon atom, n and o independently of one another are a number 1, 2, 3 or 4,
   m is a number 0 or l,
k is a number 0 or 1,
l is a number 1, 2, 3 or 4,
and their salts, their solvates and the solvates of their salts.

Particular preference is also given in the context of the present invention to compounds of formula (I) or (Ia) in which
R³ represents a group of formula

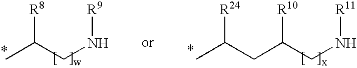

whereby
* is the linkage site to the nitrogen atom,
R⁸ represents a group of formula *—CONHR¹⁴ or *—CH₂CONHR¹⁵,
   wherein
* is the linkage site to the carbon atom,
   R¹⁴ and R¹⁵ independently of one another represent a group of formula

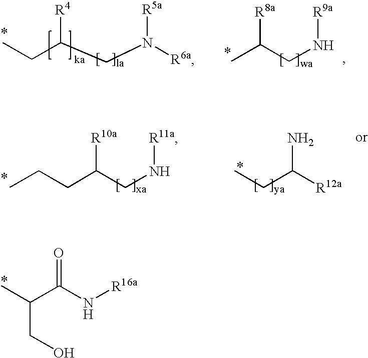

wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
   wherein
   * is the linkage site to the carbon atom,
   Z1a and Z2a independently of one another are a number 1, 2 or 3,
   R^{13a} represents hydrogen or methyl,
   and
   R^{14a} and R^{15a} independently of one another represent a group of formula

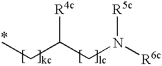

wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula

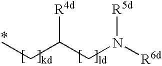

wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
R⁹ and R¹¹ independently of one another represent hydrogen, methyl, *—C(NH₂)═NH or a group of formula

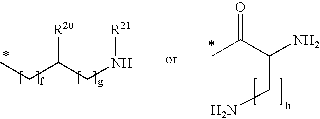

wherein
* is the linkage site to the nitrogen atom,
R²⁰ represents hydrogen or *—(CH₂)ᵢ—NHR²²,
   wherein
   R²² represents hydrogen or methyl, and
   i is a number 1, 2 or 3,
R²¹ represents hydrogen or methyl,
f is a number 0, 1, 2 or 3,
g is a number 1, 2 or 3,
and
h is a number 1, 2, 3 or 4,
or
R⁸ represents *—(CH₂)_{Z1}—OH,
   wherein
   * is the linkage site to the carbon atom,
   Z1 is a number 1, 2 or 3,
and
R⁹ represents a group of formula

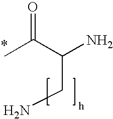

wherein
* is the linkage site to the nitrogen atom,
and
h is a number 1, 2, 3 or 4,
R¹⁰ represents amino or hydroxy,
R²⁴ represents a group of formula *—CONHR²⁵,
   wherein
   * is the linkage site to the carbon atom,
   R²⁵ represents a group of formula

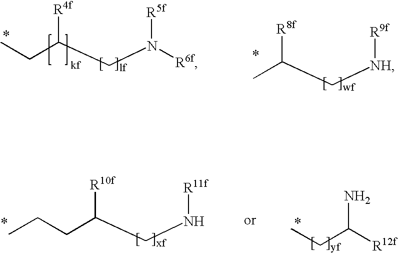

wherein
* is the linkage site to the nitrogen atom,
R^{4f} represents hydrogen, amino or hydroxy,
R^{5f} represents hydrogen, methyl or aminoethyl,
R^{6f} represents hydrogen or aminoethyl,
or
R^{5f} and R^{6f} together with the nitrogen atom to which they are bonded form a piperazine ring,
R^{8f} and R^{12f} independently of one another represent *—(CH₂)_{Z1f}—OH or *—(CH₂)_{Z2f}—NHR^{13f},
   wherein
   * is the linkage site to the carbon atom,
   R^{13f} represents hydrogen or methyl, and
   Z1f and Z2f independently of one another are a number 1, 2 or 3,
R^{9f} and R^{11f} independently of one another represent hydrogen or methyl,
R^{10f} represents amino or hydroxy,
kf is a number 0 or 1,
and
lf, wf, xf and yf independently of one another are a number 1, 2, 3 or 4,
w and x independently of one another are a number 1, 2, 3 or 4,

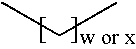

independently of one another may when w or x equals 3 carry a hydroxy group,
and their salts, their solvates and the solvates of their salts.

Preference is also given in the context of the present invention to compounds of formula (I) or (Ia) in which
R³ represents a group of formula

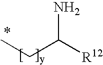

whereby
   * is the linkage site to the nitrogen atom,
R¹² represents a group of formula *—CONHR¹⁴ or *—CH₂CONHR¹⁵,
   wherein
   * is the linkage site to the carbon atom,
   R¹⁴ and R¹⁵ independently of one another represent a group of formula

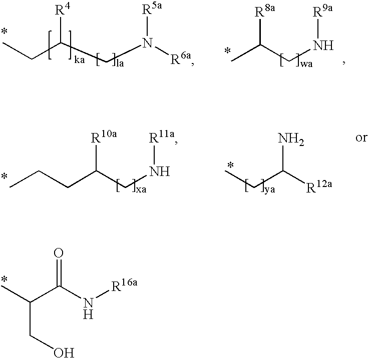

wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
   wherein
   * is the linkage site to the carbon atom,
   Z1a and Z2a independently of one another are a number 1, 2 or 3,
   R^{13a} represents hydrogen or methyl,
   and
   R^{14a} and R^{15a} independently of one another represent a group of formula

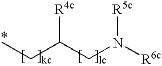

wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula

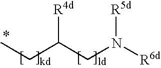

wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
y is a number 1, 2, 3 or 4,

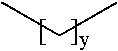

may when y equals 3 carry a hydroxy group,
and their salts, their solvates and the solvates of their salts.

Preference is also given in the context of the present invention to compounds of formula (I) or (Ia) in which
R³ represents a group of formula

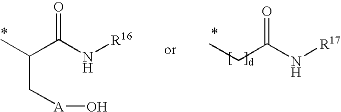

whereby
* is the linkage site to the nitrogen atom,
A represents a bond or phenyl,
R¹⁶ and R¹⁷ independently of one another represent a group of formula

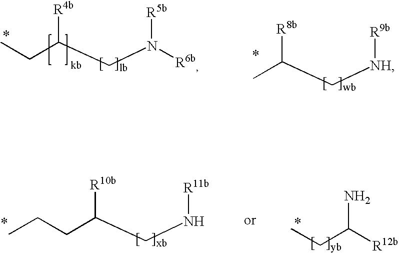

wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *—(CH₂)_{Z1b}—OH or *—(CH₂)_{Z2b}—NHR^{13b},
   wherein
   * is the linkage site to the carbon atom,
   R^{13b} represents hydrogen or methyl,
   and
   Z1b and Z2b independently of one another are a number 1, 2 or 3,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
d is a number 1, 2 or 3,
and their salts, their solvates and the solvates of their salts.

Among these, particularly preferred compounds are those in which R³ represents a group of formula

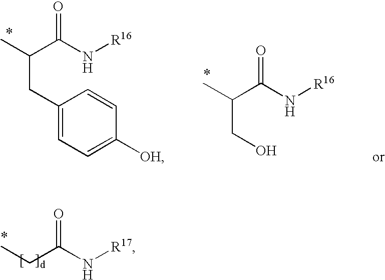

in particular a group of formula

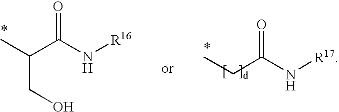

Preference is also given in the context of the present invention to compounds of or (Ia) in which
R³ represents a group of formula

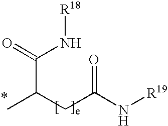

whereby
* is the linkage site to the nitrogen atom,
R¹⁸ and R¹⁹ independently of one another represent hydrogen or a group of formula

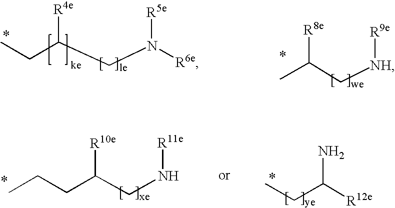

wherein
* is the linkage site to the nitrogen atom,
R^{4e} represents hydrogen, amino or hydroxy,
R^{5e} represents hydrogen, methyl or aminoethyl,
R^{6e} represents hydrogen or aminoethyl,
or
R^{5e} and R^{6e} together with the nitrogen atom to which they are bonded form a piperazine ring,
R^{8e} and R^{12e} independently of one another represent *—(CH₂)_{Z1e}—OH or *—(CH₂)_{Z2e}—NHR^{13e},
   wherein
   * is the linkage site to the carbon atom,
   R^{13e} represents hydrogen or methyl,
   and
   Z1e and Z2e independently of one another are a number 1, 2 or 3,
R^{9e} and R^{11e} independently of one another represent hydrogen or methyl,
R^{10e} represents amino or hydroxy,
ke is a number 0 or 1,
and
le, we, xe and ye independently of one another are a number 1, 2, 3 or 4,
whereby R¹⁸ and R¹⁹ are not simultaneously hydrogen,
e is a number 1, 2 or 3,
and their salts, their solvates and the solvates of their salts.

The invention further relates to a method for preparing the compounds of formula (I) or their salts, their solvates or the solvates of their salts, whereby according to method

### [A] Compounds of Formula

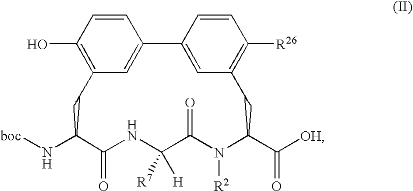

wherein R², R⁷ and R²⁶ have the meaning mentioned above, and boc is tert-butoxycarbonyl,

are reacted in a two-stage process firstly in the presence of one or more dehydrating reagents with compounds of formula

H₂NR³ (III),

wherein R³ has the abovementioned meaning,

and subsequently with an acid and/or by hydrogenolysis,

or

### [B] Compounds of Formula

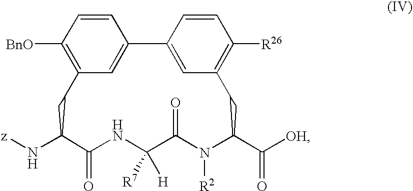

wherein R², R⁷ and R²⁶ have the meaning mentioned above, and Z is benzyloxycarbonyl, are reacted in a two-stage process firstly in the presence of one or more dehydrating reagents with compounds of formula

H₂NR³ (III),

in which R³ has the meaning mentioned above,

and subsequently with an acid or by hydrogenolysis.

The free base of the salts can be obtained for example by chromatography on a reversed phase column with an acetonitrile-water gradient with the addition of a base, in particular by using an RP18 Phenomenex Luna C18(2) column and diethylamine as base.

The invention further relates to a method for preparing the compounds of formula (I) or the solvates thereof according to claim 1 in which salts of the compounds or solvates of the salts of the compounds are converted into the compounds by chromatography with the addition of a base.

The hydroxy group on R¹ is where appropriate protected with a tert-butyldimethylsilyl group during the reaction with compounds of formula (III) which group is removed in the second reaction step.

Reactive functionalities in the radical R³ of compounds of formula (III) are introduced into the synthesis already protected, with preference for acid-labile protecting groups (e.g. boc). After reaction has taken place to give compounds of formula (I), the protecting groups can be removed by a deprotection reaction. This takes place by standard methods of protecting group chemistry. Deprotection reactions under acidic conditions or by hydrogenolysis are preferred.

The reaction in the first stage of methods [A] and [B] generally takes place in inert solvents, where appropriate in the presence of a base, preferably in a temperature range from 0° C. to 40° C. under atmospheric pressure.

Examples of suitable dehydrating reagents in this connection are carbodiimides such as, for example, N,N′-diethyl-,N,N′-dipropyl-,N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or mixtures thereof, or mixtures thereof together with bases.

Examples of bases are alkali metal carbonates such as, for example, sodium or potassium carbonate, or sodium or potassium bicarbonate, or organic bases such as trialkylamines, e.g. triethylamine, N-methylmorpholine, N-methylpiperidine, 4-dimethylaminopyridine or diisopropylethylamine.

The condensation is preferably carried out with HATU in the presence of a base, in particular diisopropylethylamine, or with EDC and HOBt in the presence of a base, in particular triethylamine.

Examples of inert solvents are halohydrocarbons such as dichloromethane or trichloromethane, hydrocarbon such as benzene, or nitromethane, dioxane, dimethylformamide or acetonitrile. It is likewise possible to employ mixtures of the solvents. Dimethylformamide is particularly preferred.

The reaction with an acid in the second stage of methods [A] and [B] preferably takes place in a temperature range from 0° C. to 40° C. under atmospheric pressure.

Suitable acids in this connection are hydrogen chloride in dioxane, hydrogen bromide in acetic acid or trifluoroacetic acid in methylene chloride.

The hydrogenolysis in the second stage of method [B] generally takes place in a solvent in the presence of hydrogen and palladium on activated carbon, preferably in a temperature range from 0° C. to 40° C. under atmospheric pressure.

Examples of solvents are alcohols such as methanol, ethanol, n-propanol or isopropanol, in a mixture with water and glacial acetic acid, with preference for a mixture of ethanol, water and glacial acetic acid.

The compounds of formula (III) are known or can be prepared in analogy to known methods.

The compounds of formula (II) are known or can be prepared by reacting compounds of formula

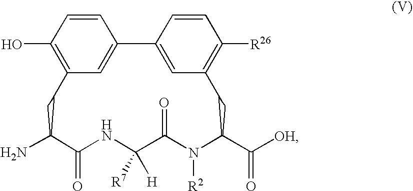

wherein R², R⁷ and R²⁶ have the meaning mentioned above, with di(tert-butyl) dicarbonate in the presence of a base.

The reaction generally takes place in a solvent, preferably in a temperature range from 0° C. to 40° C. under atmospheric pressure.

Examples of bases are alkali metal hydroxides such as sodium or potassium hydroxide, or alkali metal carbonates such as cesium carbonate, sodium or potassium carbonate, or other bases such as DBU, triethylamine or diisopropylethylamine, with preference for sodium hydroxide or sodium carbonate.

Examples of solvents are halohydrocarbons such as methylene chloride or 1,2-dichloroethane, alcohols such as methanol, ethanol or isopropanol, or water.

The reaction is preferably carried out with sodium hydroxide in water or sodium carbonate in methanol.

The compounds of formula (V) are known or can be prepared by reacting compounds of formula

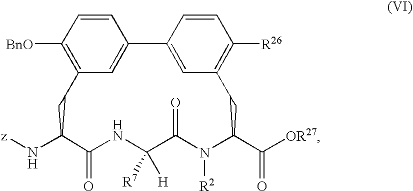

wherein R², R⁷ and R²⁶ have the meaning mentioned above, and

R²⁷ represents benzyl, methyl or ethyl,

with an acid or by hydrogenolysis as described for the second stage of method [B], where appropriate by subsequent reaction with a base to hydrolyse the methyl or ethyl ester.

The hydrolysis can for example take place as described for the reaction of compounds of formula (VI) to give compounds of formula (IV).

The compounds of formula (IV) are known or can be prepared by hydrolysing the benzyl, methyl or ethyl ester in compounds of formula (VI).

The reaction generally takes place in a solvent in the presence of a base, preferably in a temperature range from 0° C. to 40° C. under atmospheric pressure.

Examples of bases are alkali metal hydroxide such as lithium, sodium or potassium hydroxide, with preference for lithium hydroxide.

Examples of solvents are halohydrocarbons such as dichloromethane or trichloromethane, ethers, such as tetrahydrofuran or dioxane, or alcohols such as methanol, ethanol or isopropanol, or dimethylformamide. It is likewise possible to employ mixtures of the solvents or mixtures of the solvents with water. Tetrahydrofuran or a mixture of methanol and water are particularly preferred.

The compounds of formula (VI) are known or can be prepared by reacting compounds of formula

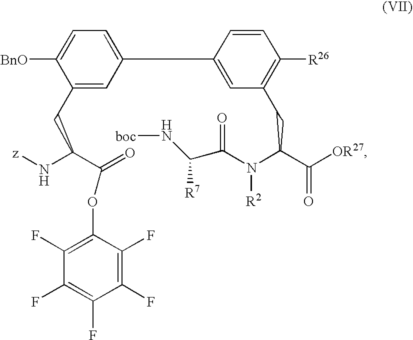

wherein R², R⁷, R²⁶ and R²⁷ have the meaning mentioned above, in the first stage with acids as described for the second stage of methods [A] and [B], and in the second stage with bases.

In the second stage the reaction with bases generally takes place in a solvent, preferably in a temperature range from 0° C. to 40° C. under atmospheric pressure.

Examples of bases are alkali metal hydroxides such as sodium or potassium hydroxide, or alkali metal carbonates such as cesium carbonate, sodium or potassium carbonate, or other bases such as DBU, triethylamine or diisopropylethylamine, with preference for triethylamine.

Examples of solvents are halohydrocarbons such as chloroform, methylene chloride or 1,2-dichloroethane, or tetrahydrofuran, or mixtures of the solvents, with preference for methylene chloride or tetrahydrofuran.

The compounds of formula (VII) are known or can be prepared by reacting compounds of formula

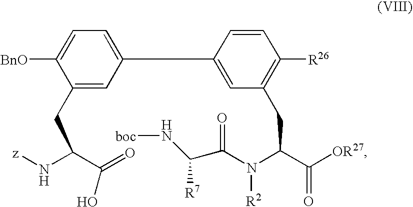

wherein R², R⁷, R²⁶ and R²⁷ have the meaning mentioned above, with pentafluorophenol in the presence of dehydrating reagents as described for the first stage of methods [A] and [B].

The reaction preferably takes place with DMAP and EDC in dichloromethane in a temperature range from −40° C. to 40° C. under atmospheric pressure.

The compounds of formula (VIII) are known or can be prepared by reacting compounds of formula

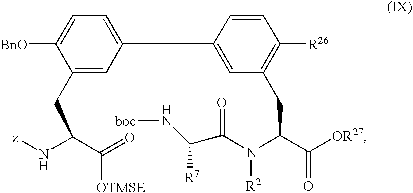

wherein R², R⁷, R²⁶ and R²⁷ have the meaning mentioned above, with fluoride, in particular with tetrabutylammonium fluoride.

The reaction generally takes place in a solvent, preferably in a temperature range from −10° C. to 30° C. under atmospheric pressure.

Examples of inert solvents are halohydrocarbons such as dichloromethane, or hydrocarbons such as benzene or toluene, or ethers such as tetrahydrofuran or dioxane, or dimethylformamide. It is likewise possible to employ mixtures of the solvents. Tetrahydrofuran and dimethylformamide are preferred solvents.

The compounds of formula (IX) are known or can be prepared by reacting compounds of formula

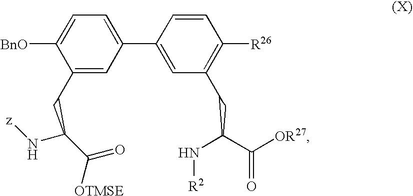

wherein R², R²⁶ and R²⁷ have the meaning mentioned above,

with compounds of formula

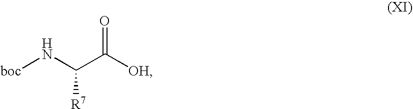

wherein R⁷ has the meaning mentioned above,
in the presence of dehydrating reagents as described for the first stage of methods [A] and [B].

The compounds of formula (X) are known or can be prepared in analogy to the methods described in the examples section.

The compounds of formula (XI) are known or can be prepared in analogy to known methods.

The compounds of the invention show a valuable range of pharmacological and pharmacokinetic effects which could not have been predicted.

They are therefore suitable for use as medicaments for the treatment and/or prophylaxis of diseases in humans and animals.

The compounds of the invention can, due to of their pharmacological properties, be employed alone or in combination with other active ingredients for the treatment and/or prophylaxis of infectious diseases, especially of bacterial infections.

For example, it is possible to treat and/or prevent local and/or systemic diseases caused by the following pathogens or by mixtures of the following pathogens: gram-positive cocci, e.g. staphylococci (Staph. aureus, Staph. epidermidis) and streptococci (Strept. agalactiae, Strept. faecalis, Strept. pneumoniae, Strept. pyogenes); gram-negative cocci (neisseria gonorrhoeae) as well as gram-negative rods such as enterobacteriaceae, e.g. Escherichia coli, Haemophilus influenzae, Citrobacter (Citrob. freundii, Citrob. divemis), Salmonella and Shigella; furthermore klebsiellas (Klebs. pneumoniae, Klebs. oxytocy), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, as well as the genus Acinetobacter. The antibacterial range additionally includes the genus Pseudomonas (Ps. aeruginosa, Ps. maltophilia) and strictly anaerobic bacteria such as Bacteroides fragilis, representatives of the genus Peptococcus, Peptostreptococcus, as well as the genus Clostridium; furthermore mycoplasmas (M. pneumoniae, M. hominis, M. urealyticum) as well as mycobacteria, e.g. Mycobacterium tuberculosis.

The above list of pathogens is merely by way of example and is by no means to be interpreted restrictively. Examples which may be mentioned of diseases which are caused by the pathogens mentioned or mixed infections and can be prevented, improved or healed by the topically applicable preparations of the invention, are:

infectious diseases in humans such as, for example, septic infections, bone and joint infections, skin infections, postoperative wound infections, abscesses, phlegmon, wound infections, infected burns, burn wounds, infections in the oral region, infections after dental operations, septic arthritis, mastitis, tonsillitis, genital infections and eye infections.

Apart from humans, bacterial infections can also be treated in other species. Examples which may be mentioned are:

Pigs: coli diarrhea, enterotoxemia, sepsis, dysentery, salmonellosis, metritis-mastitis-agalactiae syndrome, mastitis;

Ruminants (cattle, sheep, goats): diarrhea, sepsis, bronchopneumonia, salmonellosis, pasteurellosis, mycoplasmosis, genital infections;

Horses: bronchopneumonias, joint ill, puerperal and postpuerperal infections, salmonellosis;

Dogs and cats: bronchopneumonia, diarrhea, dermatitis, otitis, urinary tract infections, prostatitis;

Poultry (chickens, turkeys, quail, pigeons, ornamental birds and others): mycoplasmosis, E. coli infections, chronic airway diseases, salmonellosis, pasteurellosis, psittacosis.

It is likewise possible to treat bacterial diseases in the rearing and management of productive and ornamental fish, in which case the antibacterial spectrum is extended beyond the pathogens mentioned above to further-pathogens such as, for example, Pasteurella, Brucella, Campylobacter, Listeria, Erysipelothris, corynebacteria, Borellia, Treponema, Nocardia, Rikettsie, Yersinia.

The present invention further relates to the use of the compounds of the invention for the treatment and/or prophylaxis of diseases, preferably of bacterial diseases, especially of bacterial infections.

The present invention further relates to the use of the compounds of the invention for the treatment and/or prophylaxis of diseases, especially of the aforementioned diseases.

The present invention further relates to the use of the compounds of the invention for the production of a medicament for the treatment and/or prophylaxis of diseases, especially of the aforementioned diseases.

The present invention further relates to a method for the treatment and/or prophylaxis of diseases, especially of the aforementioned diseases, using an antibacterially effective amount of the compounds of the invention.

The compounds of the invention may act systemically and/or locally. For this purpose, they can be administered in a suitable way such as, for example, orally, parenterally, pulmonarily, nasally, sublingually, lingually, buccally, rectally, dermally, transdermally, conjuctivally or otically or as an implant or stent.

For these administration routes the compounds of the invention can be administered in suitable administration forms.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having coatings which are resistant to gastric juice or dissolve with a delay or are insoluble and control the release of the compound of the invention), tablets or films/wafers, which disintegrate rapidly in the oral cavity, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilisates or sterile powders.

Suitable for the other administration routes are, for example, pharmaceutical forms for inhalation (inter alia powder inhalers, nebulizers), nasal drops, solutions, sprays; tablets, films/wafers or capsules for lingual, sublingual or buccal administration, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, nontoxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and taste and/or odor corrigents.

The present invention further relates to medicaments which comprise at least one compound of the invention, usually together with one or more inert, nontoxic, pharmaceutically suitable excipients, and to the use thereof for the aforementioned purposes.

It has generally proved advantageous on parenteral administration to administer amounts of about 5 to 250 mg/kg of body weight per 24 h to achieve effective results. The amount on oral administration is about 5 to 100 mg/kg of body weight per 24 h.

It may nevertheless be necessary where appropriate to deviate from the stated amounts, in particular as a function of the body weight, administration route, individual behavior towards the active ingredient, nature of the preparation and time or interval over which administration takes place. Thus, it may be sufficient in some cases to make do with less than the aforementioned minimum amount, whereas in other cases the stated upper limit must be exceeded. Where larger amounts are administered, it may be advisable to divide these into a plurality of single doses over the day.

The percentage data in the following tests and examples are percentages by weight unless otherwise indicated; parts are parts by weight. Solvent ratios, dilution ratios and concentration data for liquid/liquid solutions are in each case based on volume.

### DETAILED DESCRIPTION OF PREFERRED EMBODIMENTS

### A. Examples

### Abbreviations Used

abs. absolute
aq. aqueous
Bn benzyl
boc tert-butoxycarbonyl
CDCl₃ chloroform
CH cyclohexane
d doublet (in ¹H-NMR)
dd doublet of doublets (in ¹H-NMR)
DCC dicyclohexylcarbodiimide
DIC diisopropylcarbodiimide
DIEA diisopropylethylamine (Hünig's base)
DMSO dimethyl sulfoxide

### DMAP 4-N,N-dimethylaminopyridine

DMF dimethylformamide
EA ethyl acetate (acetic acid ethyl ester)
EDC N′-(3-dimethylaminopropyl)-N-ethylcarbodiimide×HCl
ESI electrospray ionization (in MS)
Ex. example
Fmoc 9-fluorenylmethoxycarbonyl
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HOBt 1-hydroxy-1H-benzotriazole×H₂O
h hour(s)
HPLC high pressure, high performance liquid chromatography
LC-MS coupled liquid chromatography-mass spectroscopy
m multiplet (in ¹H-NMR) min minute
MS mass spectroscopy
NMR nuclear magnetic resonance spectroscopy
MTBE methyl tert-butyl ether
Pd/C palladium/carbon
PFP pentafluorophenol
q quartet (in ¹H-NMR)
R_{f} retention index (in TLC)
RP reverse phase (in HPLC)
RT room temperature
Rₜ retention time (in HPLC)
singlet (in ¹H-NMR)
sat saturated
t triplet (in ¹H-NMR)
TBS tert-butyldimethylsilyl
TFA trifluoroacetic acid

THF tetrahydrofuran

TLC thin-layer chromatography
TMSE 2-(trimethylsilyl)ethyl
TPTU 2-(2-oxo-1(2H)-pyridyl)-1,1,3,3,-tetramethyluronium tetrafluoroborate
Z benzyloxycarbonyl

### LC-MS and HPLC Methods:

Method 1 (LC-MS): Instrument: Micromass Quattro LCZ with HPLC Agilent series 1100; column: Phenomenex Synergi 2 μHydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 2 (LC-MS): MS instrument type: Micromass ZQ; HPLC instrument type: Waters Alliance 2795; column: Phenomenex Synergi 2 μl Hydro-RP Mercury 20×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 3 (LC-MS): MS instrument type: Micromass ZQ; HPLC instrument type: HP 1100 Series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4 (LC-MS): Instrument: Micromass Platform LCZ with HPLC Agilent series 1100; column: Grom-SIL1200DS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 5 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 6 (LC-MS): MS instrument type: Micromass ZQ; HPLC instrument type: HP 1100 Series; UV DAD; column: Grom-Sil 1200DS-4 HE 50 mm×2 mm, 3.0 μm; eluent A: water+500 μl of 50% formic acid/l, eluent B: acetonitrile+500 μl of 50% formic acid/l; gradient: 0.0 min 0% B→2.9 min 70% B→3.1 min 90% B→4.5 min 90% B; oven: 50° C., flow rate: 0.8 ml/min, UV detection: 210 nm.

Method 7 LC-MS): MS instrument type: Micromass ZQ; HPLC instrument type: Waters Alliance 2790; column: Grom-Sil 1200DS-4 HE 50 mm×2 mm, 3.0 μm; eluent A: water+500 μl of 50% formic acid; eluent B: acetonitrile+500 μl of 50% formic acid/l; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min 5.5 min→5.5 min 1.25 ml/min; UV detection: 210 nm.

Method 8 (LC-MS): Instrument: Micromass Platform LCZ with HPLC Agilent series 1100; column: Thermo HyPURITY Aquastar, 3μ 50 mm×2.1 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 9 (LC-MS): MS instrument type: Micromass ZQ; HPLC instrument type: Waters Alliance 2790; column: Grom-Sil 1200DS-4 HE 50×2 mm, 3.0 μm; eluent B: acetonitrile+0.05% formic acid, eluent A: water+0.05% formic acid; gradient: 0.0 min 70% B -4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 10 (LC-MS): Instrument: Micromass Platform LCZ with HPLC agilent series 1100; column: Thermo Hypersil GOLD-3μ 20×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 11 (HPLC): Instrument: HP 1100 with DAD detection; column: Kromasil RP-18, 60 mm×2 mm, 3.5 μm; eluent A: 5 ml of HClO₄/l of water, eluent B: acetonitrile; gradient: 0 min 2% B, 0.5 min 2% B, 4.5 min 90% B, 6.5 min 90% B; flow rate: 0.75 ml/min; oven: 30° C.; UV detection: 210 nm.

Method 12 (HPLC): Instrument: HP 1100 with DAD detection; column: Kromasil RP-18, 60 mm×2 mm, 3.5 μm; eluent A: 5 ml of HClO₄/l of water, eluent B: acetonitrile; gradient: 0 min 2% B, 0.5 min 2% B, 4.5 min 90% B, 15 min 90% B; flow rate: 0.75 ml/min; oven: 30° C.; UV detection: 210 nm.

### Starting Compounds

### Example 1A

### 5-Bromo-2-methylbenzaldehyde

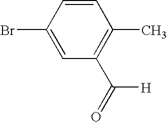

77.7 g (583 mmol) of aluminum trichloride are suspended in 200 ml of dichloromethane and cooled to 0° C. 40.0 g (333 mmol) of 2-methylbenzaldehyde are added dropwise over the course of 30 min. Then, 53.2 g (333 mmol) of bromine are added over the course of 6 h at 0° C., the mixture is allowed to warm to RT and then stirred for 12 h. The reaction solution is added to 500 ml of ice-water. The aqueous phase is extracted a number of times with dichloromethane. The combined organic phases are washed successively with 2N hydrochloric acid, a saturated aqueous sodium bicarbonate solution and a saturated aqueous sodium chloride solution. The organic phase is dried over sodium sulfate and concentrated in vacuo. The residue is purified by silica gel chromatography and then via crystallization from cyclohexane. The precipitated product is collected by filtration.

Yield: 3.2 g (5% of theory)

LC-MS (Method 7): Rₜ=3.26 min

MS (EI): m/z=199 (M+H)⁺

### Example 2A

### Methyl (2Z)-3-(3-bromophenyl)-2-[(tert-butoxycarbonyl)amino]acrylate

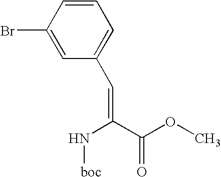

7.48 ml (59.5 mmol) of N,N,N,N-tetramethylguanidine are added to a solution, cooled to −70° C., of 10 g (54.1 mmol) of 3-bromobenzaldehyde and 17.7 g (59.5 mmol) of methyl [(tert-butoxycarbonyl)amino](dimethoxyphosphoryl)acetate in 200 ml of anhydrous tetrahydrofuran. After stirring for 4 h at −70° C., the reaction mixture is stirred for 15 h at RT. 500 ml of water and 500 ml of ethyl acetate are added to the mixture. The organic phase is washed with water, dried over sodium sulfate and concentrated. The crude product is purified by column chromatography on silica gel (mobile phase: cyclohexane:ethyl acetate 4:1).

Yield: quant.

LC-MS (Method 3): Rₜ=2.61 min.

MS (EI): m/z=356 (M+H)⁺.

¹H-NMR (300 MHz, DMSO-d₆): δ=1.40 (s, 9H), 3.73 (s, 3H), 7.15 (br.s, 1H), 7.48 (m, 1H), 7.56 (dd, 1H), 7.63 (dd, 1H), 7.86 (s, 1H), 8.82 (br.s, 1H).

Example 3A is prepared from the corresponding starting materials in analogy to the above procedure:

| | | Prepared in | |
|---|---|---|---|
| Example | | analogy to | |
| No. | Structure | Example No. | Analytical Data |
| 3A | 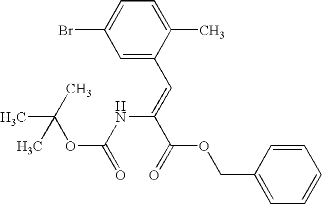 | 2Afrom Ex. IA andbenzyl [(tert-butoxycarbonyl)-amino](dimethoxy-phosphoryl)acetate | LC-MS (Method 4): Rₜ = 3.38 min.MS (EI): m/z = 446 (M + H)⁺¹H-NMR (300 MHz, CDCl₃):δ = 1.35 (s, 9H), 2.28 (s, 3H), 5.30(s, 2H), 6.21 (br. s, 1H), 7.04 (d,1H), 7.21-7.46 (m, 7H), 7.10 (d,1H). |

### Example 4A

### Methyl 3-bromo-N-(tert-butoxycarbonyl)-L-phenylalaninate

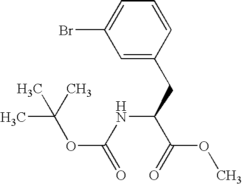

10 g (28.1 mmol) of methyl-(2Z)-3-(3-bromophenyl)-2-[(tert-butoxycarbonyl)amino]acrylate (Example 2A) are dissolved in a mixture of 150 ml of ethanol and 100 ml of dioxane. Under an argon atmosphere, 100 mg (0.14 mmol) of hydrogenation catalyst [(+)-1,2-bis((2S,5S)-2,5-diethylphospholano)benzene(cyclooctadiene)rhodium(I) trifluoromethanesulfonate] are added, and argon is passed through the solution for 30 min. Hydrogenation is then carried out for 5 days under a hydrogen pressure of 3 bar. The mixture is filtered through silica gel, and careful afterwashing with ethanol is carried out. The filtrate is concentrated in vacuo and the crude product is dried under high vacuum.

Yield: 9.2 g (89% of theory)

LC-MS (Method 3): Rₜ=2.63 min.

MS (EI): m/z=358 (M+H)⁺

¹H-NMR (400 MHz, DMSO-d₆): δ=1.32 (s, 9H), 2.74 (m_{c}, 1H), 3.03 (m_{c}, 1H), 3.62 (s, 3H), 4.70 (m_{c}, 1H), 7.20-7.5 (m, 5H).

Example 5A is prepared from the corresponding starting materials in analogy to the above procedure:

| | | Prepared in | |
|---|---|---|---|
| Example | | analogy to | |
| No. | Structure | Example No. | Analytical Data |
| 5A | 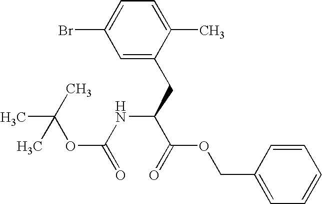 | 4Afrom Ex. 3A | LC-MS (Method 6): Rₜ = 3.81 min.MS (EI): m/z = 448 (M + H)⁺¹H-NMR (300 MHz, CDCl₃):δ = 1.39 (s, 9H), 2.24 (s, 3H), 2.83-3.15(m, 2H), 4.57 (m_{c}, 1H), 5.00 (br. s, 1H),5.09 (dd, 2H), 6.97 (d, 1H), 7.14-7.48(m, 7H). |

### Example 6A

### Methyl 3-bromo-N-(tert-butoxycarbonyl)-N-methyl-L-phenylalaninate

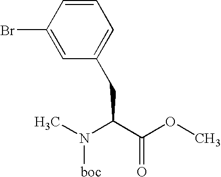

49.8 g (350.86 mmol) of iodomethane and 2.28 g (57.01 mmol) of sodium hydride are added to a solution of 16.5 g (43.86 mmol) of methyl 3-bromo-N-(tert-butoxycarbonyl)-L-phenylalaninate (Example 4A) in 220 ml of anhydrous tetrahydrofuran. The reaction mixture is stirred overnight at RT. 1000 ml of water and 1000 ml of ethyl acetate are added to the mixture. The organic phase is washed successively with water and a saturated sodium chloride solution, dried over sodium sulfate and concentrated. The crude product is purified by column chromatography on silica gel (mobile phase: cyclohexane:ethyl acetate 3:1).

Yield: quant.

HPLC (Method 11): Rₜ=5.1 min.

MS (DCI(NH₃)): m/z=390 (M+H)⁺.

¹H-NMR (400 MHz, CDCl₃): δ=1.48 (d, 9H), 2.23 (d, 3H), 3.09 (dd, 1H), 3.30 (dd, 1H), 3.75 (s, 3H), 4.70 (ddd, 1H), 6.92 (dd, 1H), 7.30 (m, 2H).

### Example 7A

### Methyl (2S)-3-(4′-(benzyloxy)-3′-{(2S)-2-{[(benzyloxy)carbonyl]amino}-3-oxo-3-[2-(trimethylsilyl)ethoxy]propyl}biphenyl-3-yl)-2-[(tert-butoxycarbonyl)amino]propanoate

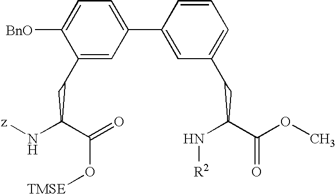

A solution of 6.0 g (16.8 mmol) of methyl 3-bromo-N-(tert-butoxycarbonyl)-N-methyl-L-phenylalaninate (Example 4A) and 11.7 g (18.4 mmol) of 2-(trimethylsilyl)ethyl-2-(benzyloxy)-N-[(benzyloxy)carbonyl]-5-(4,4,5,5-tetramethyl-1,3,2-dioxaborolan-2-yl)-L-phenylalaninate (Example 84A from WO03/106480) in 80 ml of 1-methyl-2-pyrrolidone and 4 ml of water is rendered inert and saturated with argon. 1.37 g (1.67 mmol) of bis(diphenylphosphino)ferrocenepalladium(II) chloride (PdCl₂(dppf)) and 11 g (34 mmol) of cesium carbonate are then added. Argon is gently passed over the reaction mixture, which is stirred for 10 h at 50° C. The mixture is cooled, taken up in dichloromethane and washed with water. The organic phase is dried over magnesium sulfate and the solvent is concentrated in vacuo. The residue is purified by column chromatography on silica gel (cyclohexane:ethyl acetate 15:1→7:1).

Yield: 6.82 g (52% of theory.).

LC-MS (Method 1): Rₜ=3.41 min

MS (EI): m/z=783 (M+H)⁺.

Examples 8A and 9A listed in the following table are prepared from the corresponding starting materials in analogy to the above procedure:

| | | Prepared in | |
|---|---|---|---|
| Example | | analogy to | |
| No. | Structure | Example No. | Analytical Data |
| 8A | 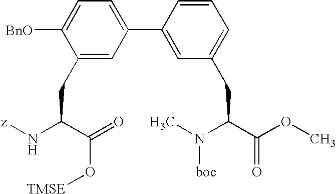 | 7Afrom Ex. 4A andEx. 84A fromWO03/106480 | HPLC (Method 12): Rₜ = 6.62 min.MS (ES): m/z = 819 (M + Na)⁺ |
| 9A | 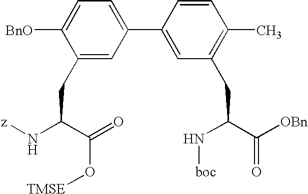 | 7Afrom Ex. 5A andEx. 84A fromWO03/106480 | LC-MS (Method 9): Rₜ = 4.01 min.MS (ES): m/z = 873 (M + H)⁺ |

### Example 10A

### Methyl (2S)-2-amino-3-(4′-(benzyloxy)-3′-{(2S)-2-{[(benzyloxy)carbonyl]amino}-3-oxo-3-[2-(trimethylsilyl)ethoxy]propyl}biphenyl-3-yl)propanoate hydrochloride

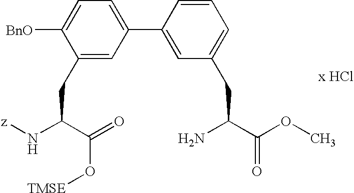

54 ml of a 4M hydrogen chloride-dioxane solution are added to a solution, cooled to 0° C., of 4.0 g (3.6 mmol) of the compound from Example 7A in 10 ml of anhydrous dioxane. After stirring for 3 h, the solvent is concentrated in vacuo, coevaporated several times with dichloromethane and dried to constant weight under high vacuum. The crude product is reacted without further purification.

Yield: quant.

LC-MS (Method 2): Rₜ=2.24 min.

MS (EI): m/z=683 (M−HCl+H)⁺.

Examples 11A and 12A listed in the following table are prepared from the corresponding starting materials in analogy to the above procedure:

| | | Prepared in | |
|---|---|---|---|
| Example | | analogy to | |
| No. | Structure | Example No. | Analytical Data |
| 11A | 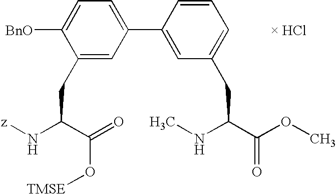 | 10Afrom Ex. 8A | Crude product was reacted withoutfurther purification |
| 12A | 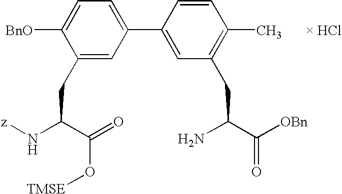 | 10Afrom Ex. 9A | LC-MS (Method 6): Rₜ = 3.10 min.MS (ES): m/z 773 (M − HCl + H)⁺ |

### Example 13A

### 2-(Trimethylsilyl)ethyl (2S)-3-(4-(benzyloxy)-3′-{(2S)-2-[((2S,4R)-5-{[(benzyloxy)carbonyl]amino}-2-[(tert-butoxycarbonyl)amino]-4-{[tert-butyl(dimethyl)silyl]oxy}pentanoyl)amino]-3-methoxy-3-oxopropyl}biphenyl-3-yl)-2-{[(benzyloxy)carbonyl]amino}propanoate

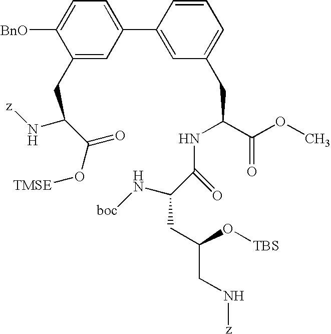

At 0° C. (bath temperature), 1.26 g (3.32 mmol) of HATU and 1.1 ml (6.2 mmol) of Hünig's base are added to a solution of 1.91 g (2.66 mmol) of the compound from Example 10A and 1.45 g (2.92 mmol) of (2S,4R)-5-{[(benzyloxy)carbonyl]amino}-2-[(tert-butoxycarbonyl)amino]-4-{[tert-butyl(dimethyl)silyl]oxy}pentanoic acid (Example 14A from WO03/106480) in 20 ml of abs. DMF. The mixture is stirred for 30 min at this temperature, then a further 0.55 ml (1.1 mmol) of Hünig's base are added and the temperature is allowed to rise to RT. After reaction overnight, everything is concentrated to dryness in vacuo and the residue is taken up in dichloromethane. The organic phase is washed with water and a saturated sodium chloride solution, dried over sodium sulfate and concentrated. The crude product is purified by chromatography on silica gel (mobile phase: cyclohexane/ethyl acetate 5:1→3:1).

Yield: 1.89 g (61% of theory)

LC-MS (Method 3): Rₜ=3.66 min.

MS (EI): m/z=1161 (M+H)⁺

### Example 14A

### 2-(Trimethylsilyl)ethyl-(2S)-3-{4-(benzyloxy)-3′-[(2S)-2-({(2S)-5-{[(benzyloxy)carbonyl]amino}-2-[(tert-butoxycarbonyl)amino]pentanoyl}amino)-3-methoxy-3-oxopropyl]biphenyl-3-yl}-2-{[(benzyloxy)carbonyl]amino}propanoate

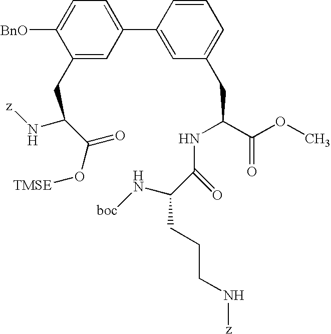

At 0° C. (bath temperature), 1.03 g (2.7 mmol) of HATU and 1.1 ml (6.1 mmol) of Hünig's base are added to a solution of 1.55 g (2.16 mmol) of the compound from Example 10A and 0.95 g (2.59 mmol) of N⁵-[(benzyloxy)carbonyl]-N²-(tert-butoxycarbonyl)-L-ornithine in 28 ml of abs. DMF. The mixture is stirred for 30 min at this temperature, then a further 0.3 ml (1.5 mmol) of Hünig's base are added and the temperature is allowed to rise to RT. After reaction overnight, everything is concentrated to dryness in vacuo and the residue is taken up in dichloromethane. The organic phase is washed with water and a saturated sodium chloride solution, dried over sodium sulfate and concentrated. The crude product is purified by chromatography on silica gel (mobile phase: dichloromethane/ethyl acetate 30:1-5:1).

Yield: 1.67 g (75% of theory)

LC-MS (Method 1): Rₜ=3.40 min.

MS (EI): m/z=1031 (M+H)⁺

Examples 15A to 17A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 15A | 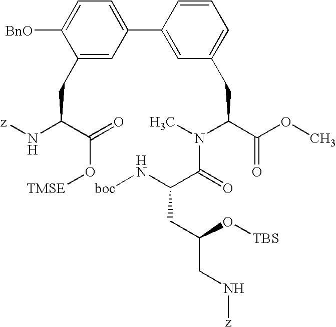 | 13Afrom Ex. 11Aand Ex. 14AfromWO03/106480 | LC-MS (Method 5): Rₜ = 3.47 min.MS (ES): m/z = 1175 (M + H)⁺ |
| 16A | 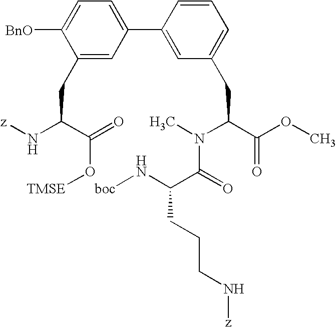 | 14Afrom Ex. 11Aand N⁵-[(benzyl-oxy)carbonyl]-N²-(tert-butoxy-carbonyl)-L-ornithine | LC-MS (Method 3): Rₜ = 3.52 min.MS (ES): m/z = 1045 (M + H)⁺ |
| 17A | 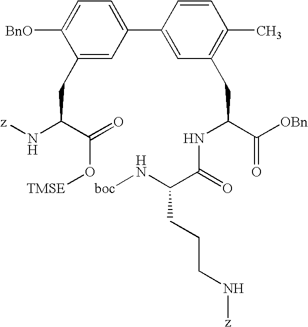 | 14Afrom Ex. 12Aand N⁵-[(Benzyl-oxy)carbonyl]-N²-(tert-butoxy-carbonyl)-L-ornithine | LC-MS (Method 3): Rₜ = 3.54 min.MS (ES): m/z = 1121 (M + H)⁺ |

### Example 18A

### (2S)-3-{4-(Benzyloxy)-3′-[(2S)-2-({(2S,4R)-5-{[(benzyloxy)carbonyl]amino}-2-[(tert-butoxycarbonyl)amino]-4-hydroxypentanoyl}amino)-3-methoxy-3-oxopropyl]-biphenyl-3-yl}-2-{[(benzyloxy)carbonyl]amino}propanoic acid

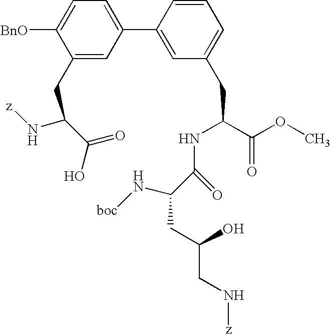

4.88 ml (4.88 mmol) of a 1N tetra-n-butylammonium fluoride solution in THF are added to a solution of 1.89 g (1.63 mmol) of the compound from Example 13A in 10 ml of abs. DMF with stirring. After 2 h at RT, the mixture is cooled to 0° C., and ice-water and some 0.5 N hydrochloric acid are added. The mixture is immediately extracted with ethyl acetate. The organic phase is dried over magnesium sulfate, concentrated in vacuo and dried under high vacuum. The crude product is reacted without further purification.

Yield: quant.

LC-MS (Method 3): Rₜ=2.90 min

MS (EI): m/z=947 (M+H)⁺

### Example 19A

(2S)-3-{4-(Benzyloxy)-3′-[(2S)-2-({(2S)-5-{[(benzyloxy)carbonyl]amino}-2-[(tert-butoxy-carbonyl)amino]pentanoyl}amino)-3-methoxy-3-oxopropyl]biphenyl-3-yl}-2-{[(benzyloxy)carbonyl]amino}propanoic acid

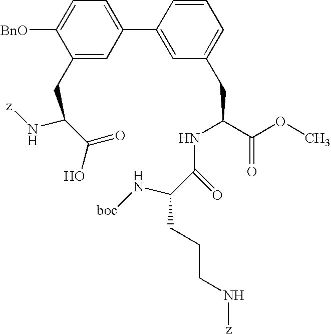

3.58 ml of a 1N tetra-n-butylammonium fluoride solution in THF are added dropwise to a solution of 2.38 g (1.79 mmol) of the compound from Example 14A in 35 ml of absolute DMF. After 2 h at RT, the mixture is cooled to 0° C., and ice-water and some 0.5 N hydrochloric acid are added. The mixture is immediately extracted with ethyl acetate. The organic phase is dried over magnesium sulfate, concentrated in vacuo and dried under high vacuum. The crude product is reacted without further purification.

Yield: quant.

LC-MS (Method 2): Rₜ=2.88 min.

MS (EI): m/z=931 (M+H)⁺.

Examples 20A to 22A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 20A | 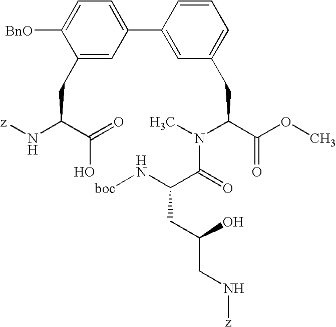 | 18Afrom Ex.15A | Crude product was reacted withoutfurther purification |
| 21A | 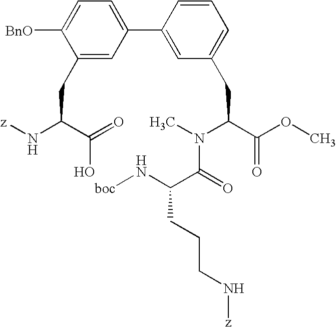 | 19Afrom Ex.16A | Crude product was reacted withoutfurther purification |
| 22A | 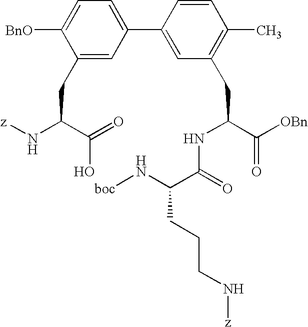 | 19Afrom Ex.17A | LC-MS (Method 6): Rₜ = 3.90 minMS (ES): m/z = 1021 (M + H)⁺ |

### Example 23A

### Pentafluorophenyl (2S)-3-{4-(benzyloxy)-3′-[(2S)-2-({(2S,4R)-5-{[(benzyloxy)carbonyl]amino}-2-[(tert-butoxycarbonyl)amino]-4-hydroxypentanoyl}amino)-3-methoxy-3-oxopropyl]biphenyl-3-yl}-2-{[(benzyloxy)carbonyl]amino}propanoate

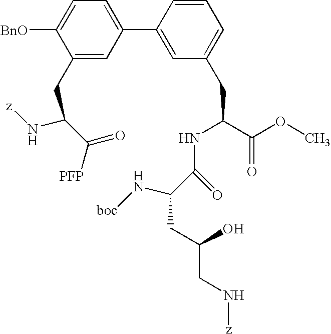

A solution of 1.54 g (1.63 mmol) of the compound from Example 18A in 50 ml of abs. dichloromethane is cooled to −20° C., and, with stirring, 1.2 g (6.52 mmol) of pentafluorophenyl, 0.02 g (0.16 mmol) of DMAP and 0.48 g (2.12 mmol) of EDC are added. The temperature is allowed to slowly rise to RT and the mixture is stirred overnight. The mixture is concentrated in vacuo and the crude product is dried to constant weight under high vacuum.

Yield: 1.8 g (99% of theory)

LC-MS (Method 2): Rₜ=3.14 min

MS (EI): m/z=1113 (M+H)⁺

### Example 24A

### Pentafluorophenyl (2S)-3-{4-(benzyloxy)-3′-[(2S)-2-({(2S)-5-{[(benzyloxy)carbonyl]-amino}-2-[(tert-butoxycarbonyl)amino]pentanoyl}amino)-3-methoxy-3-oxopropyl]biphenyl-3-yl}-2-{[(benzyloxy)carbonyl]amino}propanoate

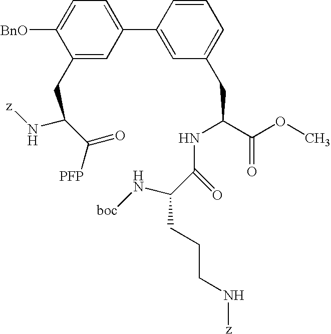

A solution of 1.67 g (1.79 mmol) of the compound from Example 19A in 70 ml of abs. dichloromethane is cooled to −20° C., and 1.65 g (8.95 mmol) of pentafluorophenyl, 0.025 g (0.18 mmol) of DMAP and 0.53 g (2.33 mmol) of EDC are added with stirring. The temperature is allowed to rise slowly to RT and the mixture is stirred overnight. The mixture is concentrated in vacuo and the crude product is dried to constant weight under high vacuum.

Yield: quant.

LC-MS (Method 3): Rₜ=3.47 min

MS (EI): m/z=1097 (M+H)⁺

Examples 25A to 27A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 25A | 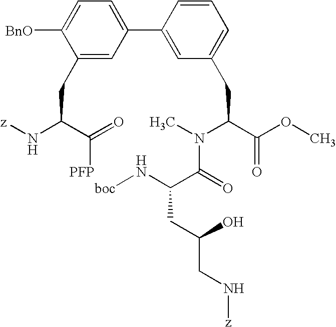 | 23Afrom Ex.20A | Crude product was reacted withoutfurther purification |
| 26A | 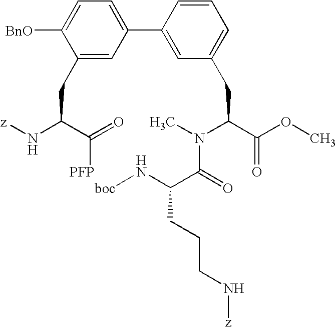 | 24Afrom Ex.21A | Crude product was reacted withoutfurther purification |
| 27A | 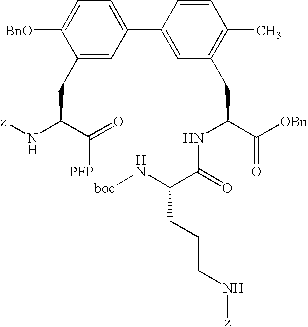 | 24Afrom Ex.22A | LC-MS (Method 5): Rₜ = 3.32 minMS (ES): m/z = 1187 (M + H)⁺ |

### Example 28A

### Methyl (2S)-2-[((2S,4R)-2-amino-5-{[(benzyloxy)carbonyl]amino}-4-hydroxypentanoyl)amino]-3-{4′-(benzyloxy)-3′-[(2S)-2-{[(benzyloxy)carbonyl]amino}-3-oxo-3-(pentafluorophenoxy)propyl]biphenyl-3-yl}propanoate hydrochloride

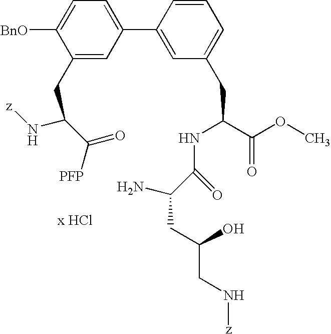

With stirring at 0° C., 20 ml of a 4N hydrogen chloride-dioxane solution are added to a solution of 1.81 g (1.63 mmol) of the compound from Example 23A in 10 ml of dioxane. The mixture is stirred for 30 min at 0° C., the temperature is allowed to rise to RT, the mixture is stirred for a further hour and then everything is concentrated to dryness in vacuo. After drying under high vacuum to constant weight the product is obtained.

Yield: quant.

LC-MS (Method 3): Rₜ=2.62 min

MS (EI): m/z=1013 (M−HCl+H)⁺

### Example 29A

### Methyl (2S)-2-[((2S)-2-amino-5-{[(benzyloxy)carbonyl]amino}pentanoyl)amino]-3-{4′-(benzyloxy)-3′-[(2S)-2-{[(benzyloxy)carbonyl]amino}-3-oxo-3-(pentafluorophenoxy)-propyl]biphenyl-3-yl}propanoate hydrochloride

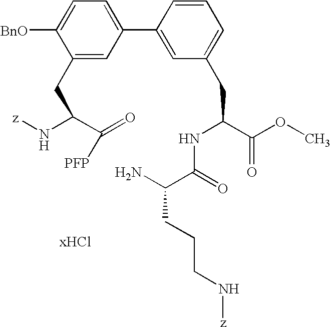

With stirring at 0° C., 60 ml of a 4N hydrogen chloride-dioxane solution are added to a solution of 1.96 g (1.79 mmol) of the compound from Example 24A in 20 ml of dioxane. The mixture is stirred for 60 min at 0° C., the temperature is allowed to rise to RT, the mixture is stirred for a further hour and then everything is concentrated to dryness in vacuo. After drying under high vacuum to constant weight the product is obtained.

Yield: quant.

LC-MS (Method 1): Rₜ=2.73 min

MS (EI): m/z=997 (M−HCl+H)⁺

Examples 30A to 32A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 30A | 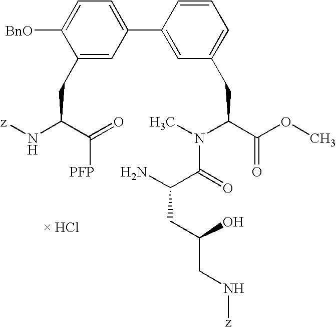 | 28Afrom Ex.25A | Crude product was reacted withoutfurther purification |
| 31A | 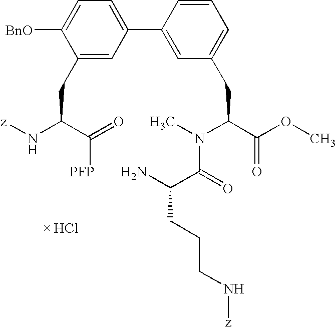 | 29Afrom Ex.26A | Crude product was reacted withoutfurther purification |
| 32A | 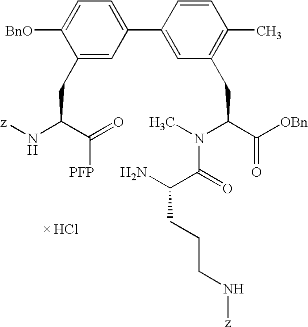 | 29Afrom Ex.27A | LC-MS (Method 5): Rₜ = 3.32 minMS (ES): m/z =1087 (M − HCl + H)⁺ |

### Example 33A

### Methyl (8S,11S,14S)-17-(benzyloxy)-14-{[(benzyloxy)carbonyl]amino}-11-((2R)-3-{[(benzyloxy)carbonyl]amino}-2-hydroxypropyl)-10,13-dioxo-9,12-diazatricyclo[14.3.1.1^{2,6}]henicosa-1(20),2(21),3,5,16,18-hexaene-8-carboxylate

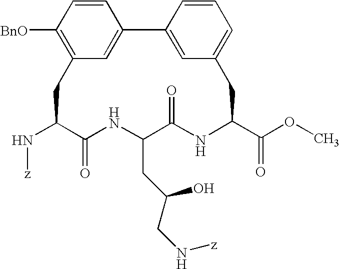

A solution of 4.5 ml (32.6 mmol) of triethylamine in 150 ml of dichloromethane is added dropwise, with vigorous stirring, to a solution of 1.71 g (1.63 mmol) of the compound from Example 28A in 600 ml of abs. dichloromethane over the course of 20 min. The mixture is stirred further overnight and then everything is concentrated in vacuo (bath temperature about 40° C.). The residue is stirred with acetonitrile and the remaining solid is collected by filtration and dried to constant weight under high vacuum.

Yield: 0.611 g (45% of theory)

LC-MS (Method 3): Rₜ=2.92 min

MS (EI): m/z=829 (M+H)⁺

### Example 34A

### Methyl (8S,11S,14S)-17-(benzyloxy)-14-{[(benzyloxy)carbonyl]amino}-1′-(3-{[(benzyloxy)carbonyl]amino}propyl)-10,13-dioxo-9,12-diazatricyclo[14.3.1.1^{2,6}]henicosa-1(20),2(21),3,5,16,18-hexaene-8-carboxylate

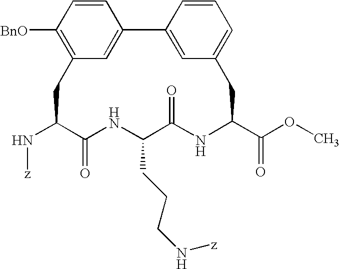

A solution of 5 ml (35.8 mmol) of triethylamine in 150 ml of chloroform is added dropwise, with vigorous stirring, to a solution of 1.85 g (1.79 mmol) of the compound from Example 29A in 600 ml of abs. chloroform over the course of 20 min. The mixture is stirred further overnight and everything is concentrated in vacuo (bath temperature about 40° C.). The residue is stirred with acetonitrile and the remaining solid is collected by filtration and dried to constant weight under high vacuum.

Yield: 1.21 g (83% of theory)

LC-MS (Method 1): Rₜ=3.0 min

MS (EI): m/z=813 (M+H)⁺

Examples 35A to 37A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 35A | 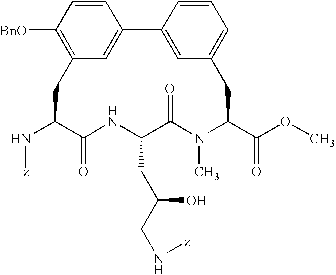 | 33Afrom Ex.30A | LC-MS (Method 2): Rₜ = 2.83 minMS (EI): m/z = 843 (M + H)⁺ |
| 36A | 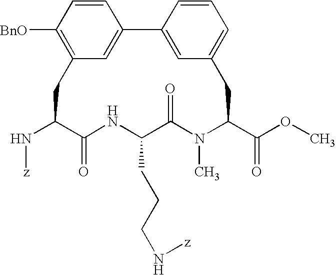 | 34Afrom Ex.31A | LC-MS (Method 3): Rₜ = 3.23 minMS (EI): m/z = 827 (M + H)⁺ |
| 37A | 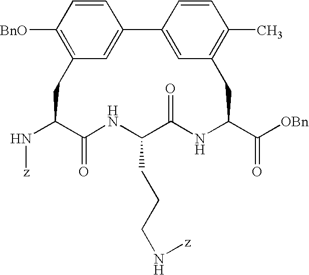 | 34Afrom Ex.32A | LC-MS (Method 1): Rₜ = 3.23 minMS (EI): m/z = 903 (M + H)⁺ |

### Example 38A

### Methyl (8S,11S,14S)-14-amino-11-[(2R)-3-amino-2-hydroxypropyl]-17-hydroxy-10,13-dioxo-9,12-diazatricyclo[14.3.1.1^{2,6}]henicosa-1(20),2(21),3,5,16,18-hexaene-8-carboxylate dihydroacetate

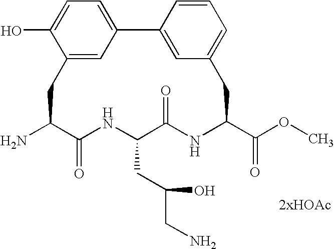

0.50 g (0.61 mmol) of the compound from Example 33A are added to a mixture of 60 ml of acetic acid/water/ethanol (4:1:1). 100 mg of palladium on activated carbon (10%) are added and the mixture is then hydrogenated for 36 h at RT under atmospheric pressure. The reaction mixture is filtered through prewashed kieselguhr, and washed with ethanol, and the filtrate is concentrated on a rotary evaporator in vacuo. The residue is dried to constant weight under high vacuum.

Yield: quant.

LC-MS (Method 2): Rₜ=0.88 min

MS (EI): m/z=471 (M-2HOAc+H)⁺.

### Example 39A

### Methyl (8S,11S,14S)-14-amino-11-(3-aminopropyl)-17-hydroxy-10,13-dioxo-9,12-diazatricyclo[14.3.1.1^{2,6}]henicosa-1(20),2(21),3,5,16,18-hexaene-8-carboxylate dihydroacetate

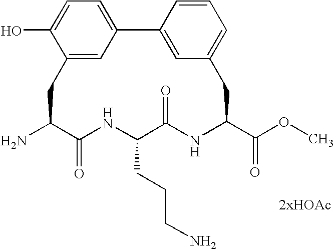

1.19 g (1.46 mmol) of the compound from Example 34A are added to a mixture of 440 ml of acetic acid/water/ethanol (4:1:1). 200 mg of palladium on activated carbon (10%) are added and the mixture is then hydrogenated for 36 h at RT under atmospheric pressure. The reaction mixture is filtered through prewashed kieselguhr, and washed with ethanol, and the filtrate is concentrated on a rotary evaporator in vacuo. The residue is dried to constant weight under high vacuum.

Yield: quant.

LC-MS (Method 8): Rₜ=2.33 min

MS (EI): m/z=455 (M-2HOAc+H)⁺.

Examples 40A to 42A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 40A | 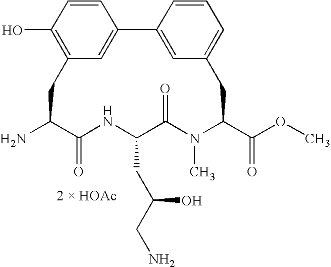 | 38Afrom Ex.35A | LC-MS (Method 3): Rₜ = 1.22 minMS (EI): m/z = 485(M − 2HOAc + H)⁺. |
| 41A | 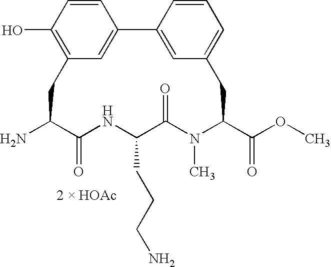 | 39Afrom Ex.36A | LC-MS (Method 10): Rₜ = 2.33 minMS (EI): m/z = 469(M − 2HOAc + H)⁺. |
| 42A | 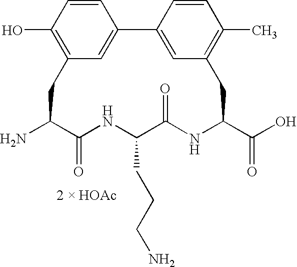 | 39Afrom Ex.37A | LC-MS (Method 2): Rₜ = 0.96 minMS (EI): m/z = 455(M − 2HOAc + H)⁺. |

### Example 43A

### 8S,11S,14S)-14-[(tert-Butoxycarbonyl)amino]-11-{(2R)-3-[(tert-butoxycarbonyl)amino]-2-hydroxypropyl}-17-hydroxy-10,13-dioxo-9,12-diazatricyclo[14.3.1^{2,6}]-henicosa-1(20),2(21),3,5,16,18-hexaene-8-carboxylic acid

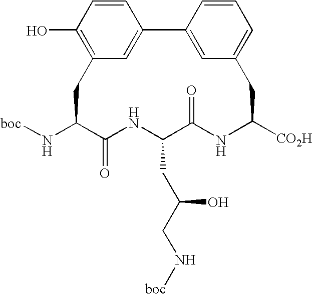

1.3 ml of a 1N sodium hydroxide solution is added to a solution of 150 mg (0.26 mmol) of the compound from Example 38A in 1 ml of water. With stirring, a solution of 170 mg (0.78 mmol) of di-tert-butyl dicarbonate in 0.5 ml of methanol is added at RT and the mixture is stirred for 4 h. The mixture is added to 15 ml of water, the pH of the mixture is adjusted to 3 using 0.1N hydrochloric acid and the mixture is extracted twice by shaking with ethyl acetate. The organic phases are combined, dried with magnesium sulfate and concentrated to dryness in vacuo. The remaining solid is purified by chromatography (Sephadex LH₂O, mobile phase: methanol/acetic acid (0.25%)).

Yield: 137 mg (81% of theory)

LC-MS (Method 1): Rₜ=1.94 min

MS (EI): m/z=657 (M+H)⁺

### Example 44A

### (8S,11S,14S)-14-[(tert-Butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaene-8-carboxylic acid

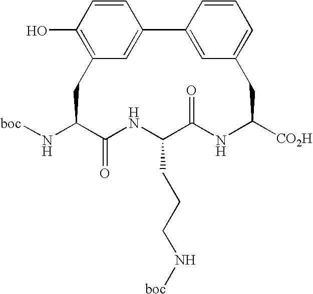

7.3 ml of a 1N sodium hydroxide solution are added to a solution of 0.85 g (1.45 mmol) of the compound from Example 39A in 5 ml of water. With stirring, a solution of 0.95 g (4.36 mmol) of di-tert-butyl dicarbonate in 2 ml of methanol is added at RT and the mixture is stirred for 6 h. The mixture is added to 25 ml of water, the pH of the mixture is adjusted to 3 using 0.1N hydrochloric acid and the mixture is extracted twice by shaking with ethyl acetate. The organic phases are combined, dried with magnesium sulfate and concentrated to dryness in vacuo. The remaining solid is purified to constant weight under high vacuum.

Yield: 0.75 g (81% of theory)

LC-MS (Method 1): Rₜ=2.20 min

MS (EI): m/z=641 (M+H)⁺

Examples 45A to 47A listed in the following table are prepared from the corresponding starting materials in analogy to the specified procedures:

| | | Prepared in | |
|---|---|---|---|
| Example | | analogy to | |
| No. | Structure | Example No. | Analytical Data |
| 45A | 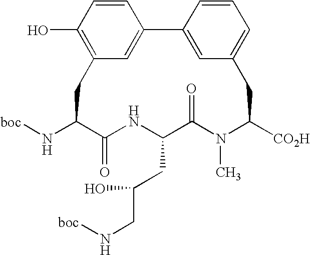 | 43Afrom Ex.40A | LC-MS (Method 2): Rₜ = 1.96 minMS (EI): m/z = 671 (M + H)⁺ |
| 46A | 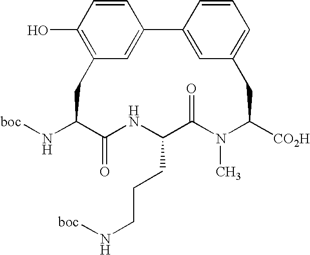 | 44Afrom Ex.41A | LC-MS (Method 2): Rₜ = 2.08 minMS (EI): m/z = 655 (M + H)⁺ |
| 47A | 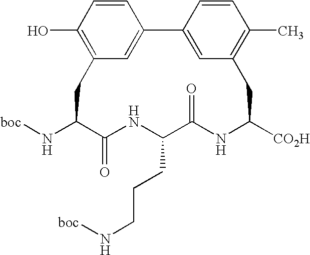 | 44Afrom Ex.42A | LC-MS (Method 2): Rₜ = 2.06 minMS (EI): m/z = 655 (M + H)⁺ |

### Example 48A

Benzyl {(1S)-4-[(tert-butoxycarbonyl)amino]-1-[({2-[(tert-butoxycarbonyl)amino]ethyl}amino)carbonyl]butyl}carbamate

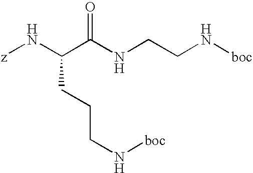

Under argon, 300 mg (0.82 mmol) of N²-[(benzyloxy)carbonyl]-N⁵-(tert-butoxycarbonyl)-L-ornithine and 171 mg (1.06 mmol) of tert-butyl-(2-aminoethyl)carbamate are dissolved in 6 ml of dimethylformamide. Then, at 0° C. (ice bath), 204 mg (1.06 mmol) of EDC and 33 mg (0.25 mmol) of HOBt are added. The mixture is slowly warmed to RT and stirred at RT for 12 h. The solution is concentrated in vacuo and the residue is taken up with ethyl acetate. The organic phase is washed successively with saturated sodium bicarbonate and sodium chloride solutions, dried over magnesium sulfate and concentrated in vacuo. The remaining solid is dried under high vacuum.

Yield: 392 mg (94% of theory)

LC-MS (Method 2): Rₜ=2.36 min

MS (ESI): m/z=509 (M+H)⁺

### Example 49A

N⁵-(tert-Butoxycarbonyl)-N-{2-[(tert-butoxycarbonyl)amino]ethyl}-L-ornithinamide

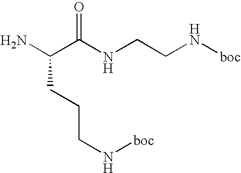

A solution of 390 mg (0.77 mmol) of benzyl {(1S)-4-[(tert-butoxycarbonyl)amino]-1-[({2-[(tert-butoxycarbonyl)amino]ethyl}amino)carbonyl]butyl}carbamate (Example 48A) in 50 ml of ethanol is hydrogenated after the addition of 40 mg of palladium on activated carbon (10%) at RT under atmospheric pressure for 4 h. The mixture is filtered through kieselguhr, and the residue is washed with ethanol. The filtrate is concentrated to dryness in vacuo. The product is reacted without further purification.

Yield: 263 mg (91% of theory)

MS (ESI): m/z=375 (M+H)⁺; 397 (M+Na)⁺.

### Example 50A

### tert-Butyl [(1S)-4-[(tert-butoxycarbonyl)amino]-1-(hydroxymethyl)butyl]carbamate

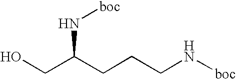

At −10° C., 91 mg (0.90 mmol) of 4-methylmorpholine and 98 mg (0.90 mmol) of ethyl chloroformate are added to a solution of 300 mg (0.90 mmol) of N²,N⁵-bis(tert-butoxycarbonyl)-L-ornithine in 10 ml of tetrahydrofuran, and the mixture is stirred for 30 min. At this temperature, 1.81 ml (1.81 mmol) of a 1M solution of lithium aluminium hydride in tetrahydrofuran are slowly added dropwise. The mixture is slowly warmed to RT and stirred at RT for 12 h. While cooling in ice, 0.1 ml of water and 0.15 ml of a 4.5% sodium hydroxide solution are cautiously added, and the mixture is stirred at RT for a further 3 h. The mixture is filtered and the filtrate is concentrated in vacuo. The residue is dissolved in ethyl acetate, washed with water, dried over magnesium sulfate and again concentrated to dryness in vacuo. The product is reacted without further purification.

Yield: 239 mg (83% of theory)

MS (ESI): m/z=319 (M+H)⁺; 341 (M+Na)⁺.

### Example 51A

### (2S)-2,5-Bis[(tert-butoxycarbonyl)amino]pentyl methanesulfonate

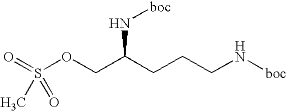

103 mg (0.90 mmol) of methanesulfonyl chloride and 0.21 ml (1.5 mmol) of triethylamine are added to a solution of 240 mg (0.75 mmol) of tert-butyl [(1S)-4-[(tert-butoxycarbonyl)amino]-1-(hydroxymethyl)butyl]carbamate (Example 50A) in 20 ml of dichloromethane, and the mixture is stirred at RT for 16 h. The mixture is diluted with dichloromethane and washed twice with 0.1N hydrochloric acid. The organic phase is dried over magnesium sulfate and concentrated to dryness in vacuo. The product is reacted without further purification.

Yield: 218 mg (73% of theory)

MS (ESI): m/z=419 (M+Na)⁺.

### Example 52A

### tert-Butyl-{(4S)-5-azido-4-[(tert-butoxycarbonyl)amino]pentyl}carbamate

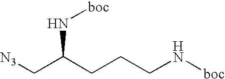

36 mg (0.55 mmol) of sodium azide are added to a solution of 218 mg (0.55 mmol) of (2S)-2,5-bis[(tert-butoxycarbonyl)amino]pentyl methanesulfonate (Example 51A) in 15 ml of dimethylformamide and the mixture is stirred at 70° C. for 12 h. Most of the solvent is distilled off in vacuo, and the residue is diluted with ethyl acetate. The mixture is washed several times with a saturated sodium bicarbonate solution, dried over magnesium sulfate and concentrated to dryness in vacuo. The product is reacted without further purification.

Yield: 188 mg (99% of theory)

MS (ESI): m/z=344 (M+H)⁺.

### Example 53A

### tert-Butyl {(4S)-5-amino-4-[(tert-butoxycarbonyl)amino]pentyl}carbamate

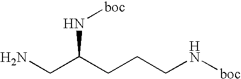

A solution of 188 mg (0.55 mmol) of tert-butyl {(4S)-5-azido-4-[(tert-butoxycarbonyl)amino]pentyl}carbamate (Example 52A) in ethanol is hydrogenated after the addition of 20 mg of palladium on activated carbon (10%) at RT under atmospheric pressure for 12 h. The mixture is filtered through kieselguhr, and the residue is washed with ethanol. The filtrate is concentrated to dryness in vacuo. The product is reacted without further purification.

Yield: 102 mg (59% of theory)

MS (ESI): m/z=318 (M+H)⁺; 340 (M+Na)⁺.

### Example 54A

Benzyl [2-({(2S)-2,5-bis[(tert-butoxycarbonyl)amino]pentyl}amino)-2-oxoethyl]carbamate

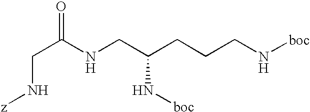

Preparation takes place in analogy to Example 48A from 92 mg (0.44 mmol) of N-[(benzyloxy)carbonyl]glycine and 181 mg (0.57 mmol) of tert-butyl {(4S)-5-amino-4-[(tert-butoxycarbonyl)amino]pentyl}carbamate (Example 53A) in 6 ml of dimethylformamide with the addition of 110 mg (0.57 mmol) of EDC and 18 mg (0.13 mmol) of HOBt. The product is purified by preparative RP-HPLC (mobile phase water/acetonitrile gradient: 90:10→5:95).

Yield: 105 mg (47% of theory)

LC-MS (Method 2): Rₜ=2.12 min.

MS (ESI): m/z=509 (M+H)⁺

### Example 55A

### tert-Butyl {(4S)-5-[(aminoacetyl)amino]-4-[(tert-butoxycarbonyl)amino]pentyl}carbamate

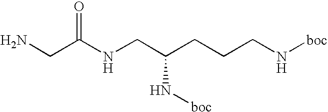

Preparation takes place in analogy to Example 49A from 105 mg (0.21 mmol) of benzyl [2-({(2S)-2,5-bis[(tert-butoxycarbonyl)amino]pentyl}amino)-2-oxoethyl]carbamate (Example 54A) in 50 ml of ethanol with the addition of 11 mg of palladium on activated carbon (10%). The product is reacted without further purification.

Yield: 64 mg (83% of theory)

MS (ESI): m/z=375 (M+H)⁺

### Example 56A

Benzyl {(1S)-1-[({(2S)-2,5-bis[(tert-butoxycarbonyl)amino]pentyl}amino)carbonyl]-4-[(tert-butoxycarbonyl)amino]butyl}carbamate

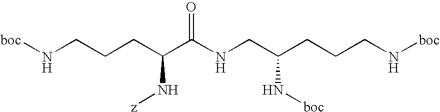

Preparation takes place in analogy to Example 48A from 120 mg (0.33 mmol) of N⁵-(tert-butoxycarbonyl)-N²-[(benzyloxy)carbonyl]-L-ornithine and 136 mg (0.43 mmol) of tert-butyl {(4S)-5-amino-4-[(tert-butoxycarbonyl)amino]pentyl}carbamate (Example 53A) in 6 ml of dimethylformamide with the addition of 82 mg (0.43 mmol) of EDC and 13 mg (0.1 mmol) of HOBt. The product is purified by preparative RP-HPLC (mobile phase water/acetonitrile gradient: 90:10→5:95).

Yield: 132 mg (61% of theory)

LC-MS (Method 3): Rₜ=2.68 min.

MS (ESI): m/z=666 (M+H)⁺

### Example 57A

### tert-Butyl [(45)-4-amino-5-({(2S)-2,5-bis[(tert-butoxycarbonyl)amino]pentyl}amino)-5-oxopentyl]carbamate

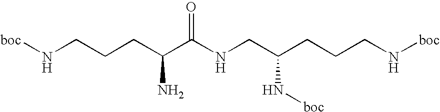

Preparation takes place in analogy to Example 49A from 132 mg (0.20 mmol) of benzyl {(1S)-1-[({(2S)-2,5-bis[(tert-butoxycarbonyl)amino]pentyl}amino)carbonyl]-4-[(tert-butoxycarbonyl)amino]butyl}carbamate (Example 56A) in 50 ml of ethanol with the addition of 13 mg of palladium on activated carbon (10%). The product is reacted without further purification.

Yield: quant.

MS (ESI): m/z=532 (M+H)⁺

### Example 58A

Benzyl [(1S)-1-[(benzyloxy)methyl]-2-({(2S)-2,5-bis[(tert-butoxycarbonyl)amino]pentyl}amino)-2-oxoethyl]carbamate

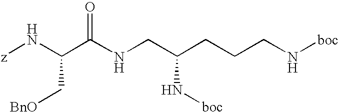

Preparation takes place in analogy to Example 48A from 150 mg (0.46 mmol) of O-benzyl-N-[(benzyloxy)carbonyl]-L-serine and 188 mg (0.59 mmol) of tert-butyl {(4S)-5-amino-4-[(tert-butoxycarbonyl)amino]pentyl}carbamate (Example 53A) in 6 ml of dimethylformamide with the addition of 114 mg (0.57 mmol) of EDC and 18 mg (0.13 mmol) of HOBt. The product is purified by preparative RP-HPLC (mobile phase water/acetonitrile gradient: 90:10→5:95).

Yield: 129 mg (45% of theory)

LC-MS (Method 3): Rₜ=2.81 min.

MS (ESI): m/z=629 (M+H)⁺

### Example 59A

### tert-Butyl {(4S)-5-{[(2S)-2-amino-3-hydroxypropanoyl]amino}-4-[(tert-butoxycarbonyl)amino]pentyl}carbamate

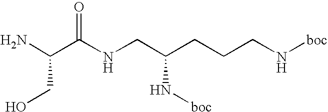

A solution of 128 mg (0.77 mmol) of benzyl [(1S)-1-[(benzyloxy)methyl]-2-({(2S)-2,5-bis[(tert-butoxycarbonyl)amino]pentyl}amino)-2-oxoethyl]carbamate (Example 58A) in 50 ml of ethanol is hydrogenated after the addition of 13 mg of palladium on activated carbon (10%) at RT under atmospheric pressure for 48 h. The mixture is filtered through kieselguhr and the residue is washed with ethanol. The filtrate is concentrated to dryness in vacuo. The product is purified by preparative RP-HPLC (mobile phase water/acetonitrile gradient: 90:10→5:95).

Yield: 22 mg (27% of theory)

LC-MS (Method 1): Rₜ=1.43 min

MS (ESI): m/z=405 (M+H)⁺

### Example 60A

### Benzyl [2-({(3S)-3-{[(benzyloxy)carbonyl]amino}-6-[(tert-butoxycarbonyl)amino]hexanoyl}amino)ethyl]carbamate

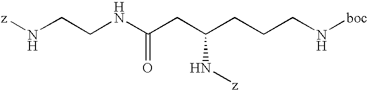

549.7 mg (1.446 mmol) of HATU and 339.7 mg (2.629 mmol) of N,N-diisopropylethylamine are added to a solution of 500 mg (1.31 mmol) of (3S)-3-{[(benzyloxy)carbonyl]amino}-6-[(tert-butoxycarbonyl)amino]hexanoic acid in 25 ml of anhydrous DMF. After stirring at RT for 15 min, 333.5 mg (1.446 mmol) of benzyl (2-aminoethyl)carbamate hydrochloride are added. The reaction mixture is stirred at RT for 15 h. The solvent is then concentrated and the residue is taken up in dichloromethane. The organic phase is washed with water, dried over magnesium sulfate and concentrated. The crude product is purified by preparative HPLC.

Yield 556.6 mg (44% of theory)

LC-MS (Method 3): Rₜ=2.41 min

MS (ESI): m/z=557 (M+H)⁺.

### Example 61A

Benzyl ((1S)-4-amino-1-{2-[(2-{[(benzyloxy)carbonyl]amino}ethyl)amino]-2-oxoethyl}butyl)carbamate hydrochloride

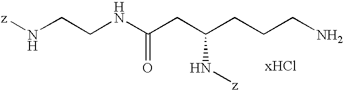

At 0° C., 8 ml of a 4M hydrogen chloride-dioxane solution are added to a solution of 320 mg (0.287 mmol) of benzyl [2-({(3S)-3-{[(benzyloxy)carbonyl]amino}-6-[(tert-butoxycarbonyl)amino]hexanoyl}amino)ethyl]carbamate (Example 60A) in 2 ml of dioxane. After 1 h at RT, the reaction solution is concentrated in vacuo, coevaporated several times with dichloromethane and dried under high vacuum. The crude product is reacted without further purification.

Yield: quant.

LC-MS (Method 2): Rₜ=2.84 min.

MS (ESI): m/z=457 (M−HCl+H)⁺.

### Example 62A

Benzyl {2-[((3S)-3-{[(benzyloxy)carbonyl]amino}-6-{[N⁵-[(benzyloxy)carbonyl]-N²-(tert-butoxycarbonyl)-L-ornithyl]amino}hexanoyl)amino]ethyl}carbamate

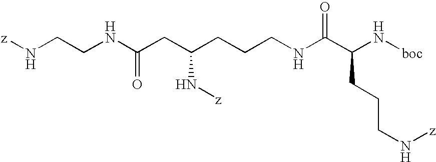

89.5 mg (0.235 mmol) of HATU and 55.3 mg (0.428 mmol) of N,N-diisopropylethylamine are added to a solution of 78.4 mg (0.214 mmol) of N⁵-[(benzyloxy)carbonyl]-N²-(tert-butoxycarbonyl)-L-ornithine in 5 ml of anhydrous DMF. After stirring at RT for 15 min, a solution of 116 mg (0.235 mmol) of benzyl ((1S)-4-amino-1-{2-[(2-{[(benzyloxy)carbonyl]amino}ethyl)amino]-2-oxoethyl}butyl)carbamate hydrochloride (Example 61A) in 5 ml of anhydrous DMF is added. The reaction mixture is stirred at RT for 15 h. The solvent is then concentrated and the residue is taken up in dichloromethane. The organic phase is washed with water, dried over magnesium sulfate and concentrated. The crude product is purified by preparative HPLC.

Yield 48 mg (28% of theory)

LC-MS (Method 2): Rₜ=2.33 min

MS (ESI): m/z=805 (M+H)⁺.

### Example 63A

Benzyl ((4S,10S)-4-amino-10-{[(benzyloxy)carbonyl]amino}-5,12,17-trioxo-19-phenyl-18-oxa-6,13,16-triazanonadec-1-yl)carbamate hydrochloride

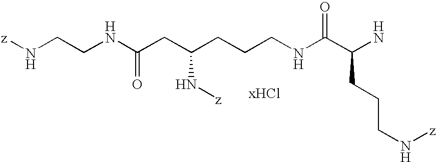

At RT, 2.5 ml of a 4M hydrogen chloride-dioxane solution are added to a solution of 48 mg (0.060 mmol) of benzyl {2-[((3S)-3-{[(benzyloxy)carbonyl]amino}-6-{[N⁵-[(benzyloxy)carbonyl]-N²-(tert-butoxycarbonyl)-L-ornithyl]amino}hexanoyl)amino]ethyl}carbamate (Example 62A) in 1 ml of dioxane. After 4 h at RT, the reaction solution is concentrated in vacuo, coevaporated several times with dichloromethane and dried under high vacuum. The crude product is reacted without further purification.

Yield: quant.

LC-MS (Method 2): Rₜ=1.69 min

MS (ESI): m/z=705 (M−HCl+H)⁺.

### Example 64A

Benzyl [(5S)-5-[(tert-butoxycarbonyl)amino]-7-({2-[(tert-butoxycarbonyl)amino]ethyl}amino)-7-oxoheptyl]carbamate

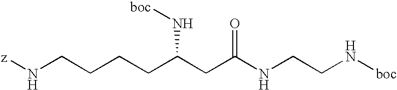

Under argon, 1 g (2.54 mmol) of (3S)-7-{[(benzyloxy)carbonyl]amino}-3-[(tert-butoxycarbonyl)amino]heptanecarboxylic acid, 406 mg (2.54 mmol) of tert-butyl (2-aminoethyl)carbamate and 0.96 ml of triethylamine (6.85 mmol) are dissolved in 20 ml of dimethylformamide. Then, at 0° C. (ice bath), 826 mg (4.3 mmol) of EDC and 113 mg (0.84 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried under high vacuum.

Yield: quant.

LC-MS (Method 2): Rₜ=2.21 min.

MS (ESI): m/z=537 (M+H)⁺

### Example 65A

### tert-Butyl ((1S)-5-amino-1-{2-[(2-{[(benzyloxy)carbonyl]amino}ethyl)amino]-2-oxoethyl}pentyl)carbamate hydroacetate

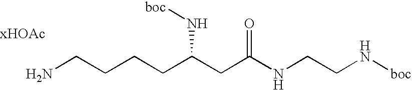

1.3 g (2.42 mmol) of benzyl [(5S)-5-[(tert-butoxycarbonyl)amino]-7-({2-[(tert-butoxycarbonyl)amino]ethyl}amino)-7-oxoheptyl]carbamate (Example 64A) are dissolved in 100 ml of a glacial acetic acid/water mixture 4/1. 70 mg of palladium on activated carbon (10%) are added thereto, and the mixture is then hydrogenated under atmospheric pressure for 15 h. The reaction mixture is filtered through prewashed kieselguhr and the filtrate is concentrated on a rotary evaporator in vacuo. The crude product is reacted without further purification.

Yield: quant.

LC-MS (Method 1): Rₜ=1.35 min.

MS (ESI): m/z=403 (M−HOAc+H)⁺

### Example 66A

Benzyl tert-butyl[(2S)-3-({(2S)-2,5-bis[(tert-butoxycarbonyl)amino]pentyl}amino)-3-oxopropane-1,2-diyl]biscarbamate

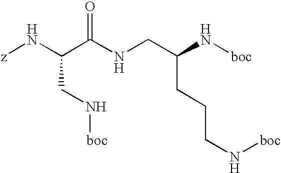

Under argon, 0.127 g (0.37 mmol) of N-[(benzyloxy)carbonyl]-3-[(tert-butoxycarbonyl)amino]-L-alanine and 0.193 g (0.49 mmol) of tert-butyl {(4S)-5-amino-4-[(tert-butoxycarbonyl)amino]pentyl}carbamate (Example 53A) are dissolved in 6 ml of dimethylformamide. Then, at 0° C. (ice bath), 0.093 g (0.419 mmol) of EDC and 0.015 g (0.11 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is purified by preparative HPLC (Kromasil, mobile phase acetonitrile/0.25% aqueous trifluoroacetic acid 5:95→95:5).

Yield: 0.126 g (53% of theory)

LC-MS (Method 1): Rₜ=2.65 min.

MS (ESI): m/z=638 (M+H)⁺

### Example 67A

### tert-Butyl [(2S)-2-amino-3-({(2S)-2,5-bis[(tert-butoxycarbonyl)amino]pentyl}amino)-3-oxopropyl]carbamate

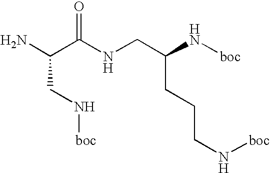

20 mg of palladium on activated carbon (10%) are added to a mixture of 0.122 g (0.19 mmol) of the compound from Example 66A in 50 ml of ethanol, and the mixture is then hydrogenated under atmospheric pressure for 4 h. The reaction mixture is filtered through kieselguhr, and the filtrate is concentrated in vacuo and dried under high vacuum. The crude product is reacted without further purification.

Yield: quant.

MS (ESI): m/z=504 (M+H)⁺

### Example 68A

Benzyl {(1S)-4-[(tert-butoxycarbonyl)amino]-1-[2-({2-[(tert-butoxycarbonyl)amino]ethyl}amino)-2-oxoethyl]butyl}carbamate

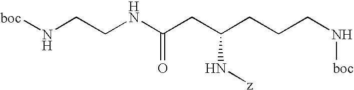

836.5 mg (2.2 mmol) of HATU and 517.0 mg (4 mmol) of N,N-diisopropylethylamine are added to a solution of 760.9 mg (2 mmol) of (3S)-3-{[(benzyloxy)carbonyl]amino}-6-[(tert-butoxycarbonyl)amino]hexanoic acid in 25 ml of anhydrous DMF. After stirring at RT for 15 min, 352.5 mg (2.2 mmol) of tert-butyl (2-aminoethyl)carbamate hydrochloride are added. The reaction mixture is stirred at RT for 15 h. The solvent is then concentrated and the residue is taken up in dichloromethane. The organic phase is washed with water, dried over magnesium sulfate and concentrated. The crude product is purified by preparative HPLC.

Yield 400 mg (38% of theory)

LC-MS (Method 1): Rₜ=2.33 min

MS (EI): m/z=523 (M+H)⁺.

### Example 69A

### tert-Butyl [(4S)-4-amino-6-({2-[(tert-butoxycarbonyl)amino]ethyl}amino)-6-oxohexyl]carbamate

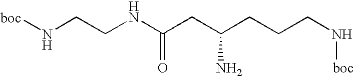

400 mg (0.765 mmol) of benzyl {(1S)-4-[(tert-butoxycarbonyl)amino]-1-[2-({2-[(tert-butoxycarbonyl)amino]ethyl}amino)-2-oxoethyl]butyl}carbamate (Example 68A) are dissolved in 50 ml of ethanol. 80 mg of palladium on activated carbon (10%) are added thereto, and the mixture is then hydrogenated under atmospheric pressure for 15 h. The reaction mixture is filtered through prewashed kieselguhr, and the filtrate is concentrated on a rotary evaporator in vacuo. The crude product is reacted without further purification.

Yield: quant.

LC-MS (Method 3): Rₜ=1.42 min

MS (ESI): m/z=389 (M+H)⁺.

### Example 70A

### Benzyl ((1S,4S)-1,4-bis{3-[(tert-butoxycarbonyl)amino]propyl}-13,13-dimethyl-2,6,11-trioxo-12-oxa-3,7,10-triazatetradec-1-yl)carbamate

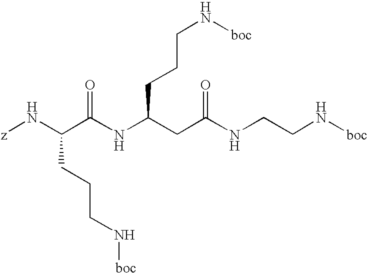

Under argon, 72 mg (0.197 mmol) of N²-[(benzyloxy)carbonyl]-N⁵-(tert-butoxycarbonyl)-L-ornithine and 100 mg (0.26 mmol) of the compound from Example 69A are dissolved in 8 ml of dimethylformamide. Then, at 0° C. (ice bath), 49 mg (0.26 mmol) of EDC and 8 mg (0.059 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried under high vacuum.

Yield 121 mg (83% of theory)

LC-MS (Method 1): Rₜ=2.24 min

MS (ESI): m/z=737 (M+H)⁺.

### Example 71A

### tert-Butyl [(4S)-4-({(2S)-2-amino-5-[(tert-butoxycarbonyl)amino]pentanoyl}amino)-6-({2-[(tert-butoxycarbonyl)amino]ethyl}amino)-6-oxohexyl]carbamate

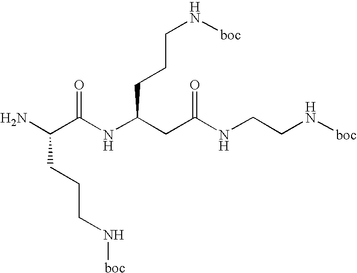

120 mg (0.16 mmol) of the compound from Example 70A are dissolved in 10 ml of ethanol. 15 mg of palladium on activated carbon (10%) are added thereto, and the mixture is then hydrogenated under atmospheric pressure for 15 h. The reaction mixture is filtered through prewashed kieselguhr and the filtrate is concentrated on a rotary evaporator in vacuo. The crude product is reacted without further purification.

Yield: quant.

MS (ESI): m/z=603 (M+H)⁺.

### Example 72A

### Benzyl [(4S)-4-[(tert-butoxycarbonyl)amino]-6-({2-[(tert-butoxycarbonyl)amino]ethyl}amino)-6-oxohexyl]carbamate

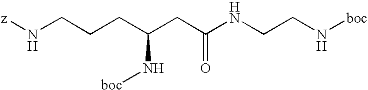

Under argon, 100 mg (0.26 mmol) of (3S)-6-{[(Benzyloxy)carbonyl]amino}-3-[(tert-butoxycarbonyl)amino]hexanoic acid and 55 mg (0.34 mmol) of tert-butyl (2-aminoethyl)carbamate are dissolved in 6 ml of dimethylformamide. Then, at 0° C. (ice bath), 66 mg (0.34 mmol) of EDC and 11 mg (0.08 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried under high vacuum.

Yield: 71 mg (51% of theory)

LC-MS (Method 3): Rₜ=2.43 min

MS (ESI): m/z=523 (M+H)⁺

### Example 73A

### tert-Butyl {(1S)-4-amino-1-[2-({2-[(tert-butoxycarbonyl)amino]ethyl}amino)-2-oxoethyl]butyl}carbamate

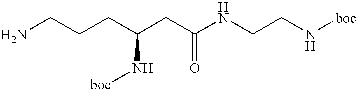

A solution of 71 mg (0.135 mmol) of the compound from Example 72A in 10 ml of ethanol is hydrogenated, after the addition of 15 mg of palladium on activated carbon (10%), for 12 h at RT under atmospheric pressure. The mixture is filtered through kieselguhr and the residue is washed with ethanol. The filtrate is concentrated to dryness in vacuo. The product is reacted without further purification.

Yield: quant.

MS (ESI): m/z=389 (M+H)⁺.

### Example 74A

Benzyl ((1S,7S)-7-[(tert-butoxycarbonyl)amino]-1-{3-[(tert-butoxycarbonyl)amino]propyl}-16,16-dimethyl-2,9,14-trioxo-15-oxa-3,10,13-triazaheptadec-1-yl)carbamate

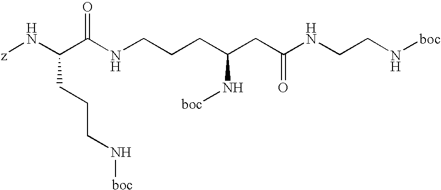

Under argon, 40 mg (0.11 mmol) of N²-[(benzyloxy)carbonyl]-N⁵-(tert-butoxycarbonyl)-L-ornithine and 55 mg (0.14 mmol) of the compound from Example 73A are dissolved in 8 ml of dimethylformamide. Then, at 0° C. (ice bath), 27 mg (0.14 mmol) of EDC and 4.4 mg (0.033 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried under high vacuum.

Yield: 72 mg (89% of theory)

LC-MS (Method 1): Rₜ=2.2 min

MS (ESI): m/z=737 (M+H)⁺

### Example 75A

### tert-Butyl {(4S,10S)-4-amino-10-[(tert-butoxycarbonyl)amino]-19,19-dimethyl-5,12,17-trioxo-18-oxa-6,13,16-triazaicos-1-yl}carbamate

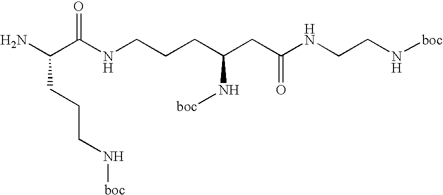

A solution of 72 mg (0.097 mmol) of the compound from Example 74A in 10 ml of ethanol is hydrogenated, after the addition of 10 mg of palladium on activated carbon (10%), for 12 h at RT under atmospheric pressure. The mixture is filtered through kieselguhr and the residue is washed with ethanol. The filtrate is concentrated to dryness in vacuo. The product is reacted without further purification.

Yield: quant.

MS (ESI): m/z=603 (M+H)⁺.

### Example 76A

Benzyl {(4S)-6-({(2S)-2,5-bis[(tert-butoxycarbonyl)amino]pentyl}amino)-4-[(tert-butoxycarbonyl)amino]-6-oxohexyl}carbamate

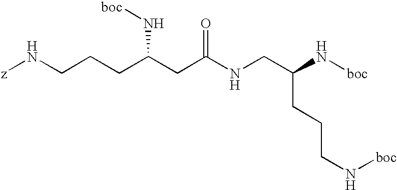

Under argon, 0.1 g (0.263 mmol) of (3S)-6-{[(benzyloxy)carbonyl]amino}-3-[(tert-butoxycarbonyl)amino]hexanecarboxylic acid (Bioorg. Med. Chem. Lett. 1998, 8, 1477-1482) and 0.108 g (0.342 mmol) of tert-butyl {(4S)-5-amino-4-[(tert-butoxycarbonyl)amino]pentyl}carbamate (Example 53A) are dissolved in 6 ml of dimethylformamide. Then, at 0° C. (ice bath), 0.066 g (0.342 mmol) of EDC and 0.011 g (0.079 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried to constant weight under high vacuum.

Yield: 0.127 g (71% of theory)

LC-MS (Method 1): Rₜ=2.36 min

MS (ESI): m/z=680 (M+H)⁺

### Example 77A

### tert-Butyl {(1S)-4-amino-1-[2-({(2S)-2,5-bis[(tert-butoxycarbonyl)amino]pentyl}amino)-2-oxoethyl]butyl}carbamate

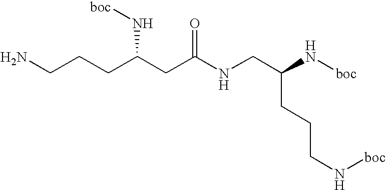

20 mg of palladium on activated carbon (10%) are added to a mixture of 0.127 g (0.19 mmol) of the compound from Example 76A in 10 ml of ethanol, and the mixture is then hydrogenated for 12 h under atmospheric pressure. The reaction mixture is filtered through kieselguhr, the filtrate is concentrated in vacuo and dried under high vacuum. The crude product is reacted without further purification.

Yield: quant.

MS (ESI): m/z=546 (M+H)⁺

### Example 78A

Benzyl ((1S,7S,12S)-7,12-bis[(tert-butoxycarbonyl)amino]-1-{3-[(tert-butoxycarbonyl)amino]propyl}-19,19-dimethyl-2,9,17-trioxo-18-oxa-3,10,16-triazaicos-1-yl)carbamate

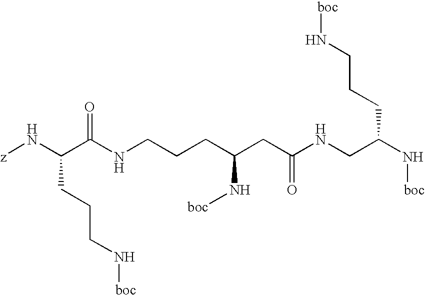

Under argon, 44 mg (0.12 mmol) of N²-[(benzyloxy)carbonyl]-N⁵-(tert-butoxycarbonyl)-L-ornithine and 85 mg (0.16 mmol) of the compound from Example 77A are dissolved in 8 ml of dimethylformamide. Then, at 0° C. (ice bath), 30 mg (0.16 mmol) of EDC and 4.9 mg (0.036 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried under high vacuum.

Yield: 91 mg (85% of theory)

LC-MS (Method 1): Rₜ=2.35 min.

MS (ESI): m/z=894 (M+H)⁺

### Example 79A

### tert-Butyl {(4S,10S,15S)-4-amino-10,15-bis[(tert-butoxycarbonyl)amino]-22,22-dimethyl-5,12,20-trioxo-21-oxa-6,13,19-triazatricos-1-yl}carbamate

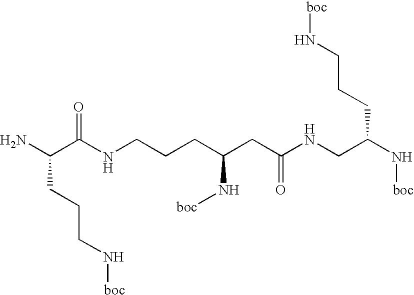

A solution of 91 mg (0.10 mmol) of the compound from Example 78A in 10 ml of ethanol is hydrogenated, after the addition of 10 mg of palladium on activated carbon (10%), for 12 h at RT under atmospheric pressure. The mixture is filtered through kieselguhr and the residue is washed with ethanol. The filtrate is concentrated to dryness in vacuo. The product is reacted without further purification.

Yield: quant.

MS (ESI): m/z=760 (M+H)⁺.

### Example 80A

Benzyl {(1S)-1-[2-({(25)-2,5-bis[(tert-butoxycarbonyl)amino]pentyl}amino)-2-oxoethyl]-4-[(tert-butoxycarbonyl)amino]butyl}carbamate

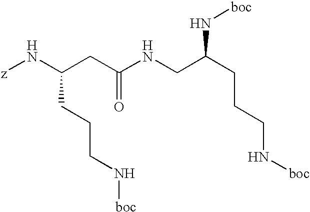

Under argon, 0.1 g (0.26 mmol) of (3S)-3-{[(benzyloxy)carbonyl]amino}-6-[(tert-butoxycarbonyl)amino]hexanoic acid (J. Med. Chem. 2002, 45, 4246-4253) and 0.11 g (0.34 mmol) of tert-butyl {(4S)-5-amino-4-[(tert-butoxycarbonyl)amino]pentyl}carbamate (Example 53A) are dissolved in 6 ml of dimethylformamide. Then, at 0° C. (ice bath), 0.065 g (0.34 mmol) of EDC and 0.011 g (0.079 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried to constant weight under high vacuum.

Yield: 0.146 g (82% of theory)

LC-MS (Method 2): Rₜ=2.5 min

MS (ESI): m/z=680 (M+H)⁺

### Example 81A

### tert-Butyl [(4S)-4-amino-6-({(2S)-2,5-bis[(tert-butoxycarbonyl)amino]pentyl}amino)-6-oxohexyl]carbamate

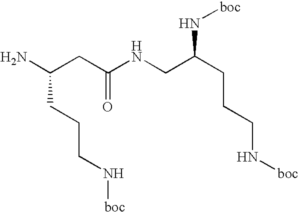

22 mg of palladium on activated carbon (10%) are added to a mixture of 0.146 g (0.22 mmol) of the compound from Example 80A in 10 ml of ethanol, and the mixture is then hydrogenated under atmospheric pressure for 12 h. The reaction mixture is filtered through kieselguhr, the filtrate is concentrated in vacuo and dried under high vacuum. The crude product is reacted without further purification.

Yield: quant.

MS (ESI): m/z=546 (M+H)⁺

### Example 82A

### Benzyl ((1S,4S,9S)-9-[(tert-butoxycarbonyl)amino]-1,4-bis{3-[(tert-butoxycarbonyl)amino]propyl}-16,16-dimethyl-2,6,14-trioxo-15-oxa-3,7,13-triazaheptadec-1-yl)carbamate

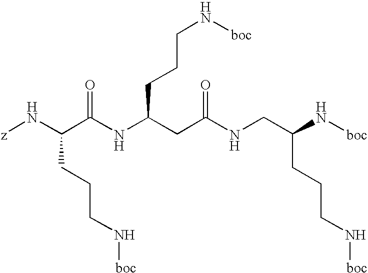

Under argon, 40 mg (0.11 mmol) of N²-[(benzyloxy)carbonyl]-N⁵-(tert-butoxycarbonyl)-L-ornithine and 77 mg (0.14 mmol) of the compound from Example 81A are dissolved in 8 ml of dimethylformamide. Then, at 0° C. (ice bath), 27 mg (0.14 mmol) of EDC and 4.4 mg (0.032 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried under high vacuum.

Yield: 78 mg (81% of theory)

LC-MS (Method 1): Rₜ=2.43 min

MS (ESI): m/z=894 (M+H)⁺

### Example 83A

### tert-Butyl ((1S,6S,9S)-9-amino-1,6-bis{3-[(tert-butoxycarbonyl)amino]propyl}-16,16-dimethyl-4,8,14-trioxo-15-oxa-3,7,13-triazaheptadec-1-yl)carbamate

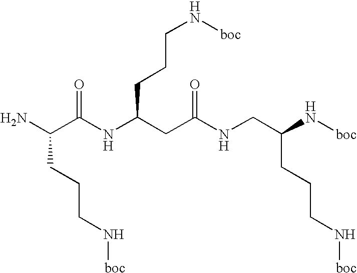

A solution of 78 mg (0.088 mmol) of the compound from Example 82A in 10 ml of ethanol is hydrogenated, after the addition of 10 mg of palladium on activated carbon (10%), for 12 h at RT under atmospheric pressure. The mixture is filtered through kieselguhr and the residue is washed with ethanol. The filtrate is concentrated to dryness in vacuo. The product is reacted without further purification.

Yield: quant.

MS (ESI): m/z=760 (M+H)⁺.

### Example 84A

### N⁵-[N²-[(Benzyloxy)carbonyl]-N⁵-(tert-butoxycarbonyl)-D-ornithyl]-N²-(tert-butoxycarbonyl)-N-{2-[(tert-butoxycarbonyl)amino]ethyl}-L-ornithinamide

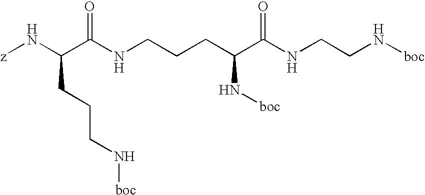

Under argon, 286 mg (0.78 mmol) of N²-[(benzyloxy)carbonyl]-N⁵-(tert-butoxycarbonyl)-D-ornithine and 439 mg (1.17 mmol) of the compound from Example 104A are dissolved in 16 ml of dimethylformamide. Then, at 0° C. (ice bath), 255 mg (1.33 mmol) of EDC and 106 mg (0.78 mmol) of HOBt are added. The mixture is slowly warmed to RT and stirred at RT for 48 h. The solution is concentrated in vacuo and the residue is taken up in dichloromethane and washed with a saturated aqueous sodium bicarbonate solution, 0.1 N hydrochloric acid and water. The combined organic phases are concentrated in vacuo and the solid obtained in this way is reacted further without purification.

Yield: 0.58 g (quant.)

LC-MS (Method 3): Rₜ=2.59 min.

MS (ESI): m/z=723 (M+H)⁺

### Example 85A

### N⁵-[N⁵-(tert-Butoxycarbonyl)-D-ornithyl]-N²-(tert-butoxycarbonyl)-N-{2-[(tert-butoxycarbonyl)amino]ethyl}-L-ornithinamide

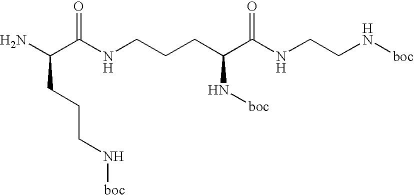

0.58 g (0.80 mmol) of the compound from Example 84A are dissolved in 27 ml of ethanol, and 0.06 g (0.06 mmol) of Pd/C are added. The mixture is hydrogenated under atmospheric pressure for 12 h and filtered through celite, and the filtrate is concentrated in vacuo. The solid obtained in this way is reacted further without purification.

Yield: 0.47 g (97% of theory)

LC-MS (Method 1): Rₜ=1.61 min.

MS (ESI): m/z=589 (M+H)⁺

### Example 86A

Benzyl [(2S)-2-[(tert-butoxycarbonyl)amino]-3-({2-[(tert-butoxycarbonyl)amino]ethyl}amino)-3-oxopropyl]carbamate

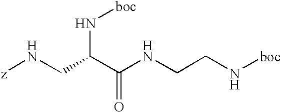

Under argon, 0.50 g (0.96 mmol) of 3-{[(benzyloxy)carbonyl]amino}-N-(tert-butoxycarbonyl)-L-alanine-N-cyclohexylcyclohexanamine (1:1) and 0.154 g (0.96 mmol) of tert-butyl (2-aminoethyl)carbamate are dissolved in 10 ml of dimethylformamide and 0.5 ml of triethylamine. Then, at 0° C. (ice bath), 0.314 g (1.64 mmol) of EDC and 0.043 g (0.32 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried to constant weight under high vacuum.

Yield: 0.41 g (88% of theory)

LC-MS (Method 2): Rₜ=2.17 min

MS (ESI): m/z=481 (M+H)⁺

### Example 87A

### 3-Amino-N²-(tert-butoxycarbonyl)-N-{2-[(tert-butoxycarbonyl)amino]ethyl}-L-alaninamide hydroacetate

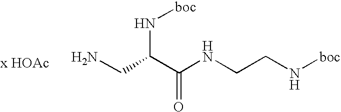

50 mg of palladium on activated carbon (10%) are added to a mixture of 0.41 g (0.847 mmol) of the compound from Example 86A in 80 ml of acetic acid/ethanol/water (4:1:1), and the mixture is then hydrogenated under atmospheric pressure for 12 h. The reaction mixture is filtered through kieselguhr, and the filtrate is concentrated in vacuo and dried under high vacuum. The crude product is reacted without further purification.

Yield: quant.

LC-MS (Method 2): Rₜ=1.09 min

MS (ESI): m/z=347 (M−HOAc+H)⁺

### Example 88A

### N⁵-{N-[(Benzyloxy)carbonyl]glycyl}-N²-(tert-butoxycarbonyl)-N-{2-[(tert-butoxycarbonyl)amino]ethyl}-L-ornithinamide

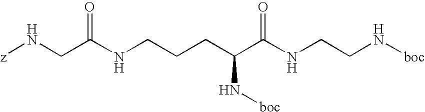

Under argon, 300 mg (1.43 mmol) of N-[(benzyloxy)carbonyl]glycine and 830 mg (2.15 mmol) of the compound from Example 104A are dissolved in 28 ml of dimethylformamide. Then, at 0° C. (ice bath), 467 mg (2.44 mmol) of EDC and 194 mg (1.43 mmol) of HOBt are added. The mixture is slowly warmed to RT and stirred at RT for 48 h. The solution is concentrated in vacuo and the residue is taken up in dichloromethane and washed with a saturated sodium bicarbonate solution, 0.1N hydrochloric acid and water. The combined organic phases are concentrated in vacuo, and the solid obtained in this way is reacted further without purification.

Yield: quant.

LC-MS (Method 2): Rₜ=1.98 min.

MS (ESI): m/z=566 (M+H)⁺

### Example 89A

### N⁵-Glycyl-N²-(tert-butoxycarbonyl)-N-{2-[(tert-butoxycarbonyl)amino]ethyl}-L-ornithinamide

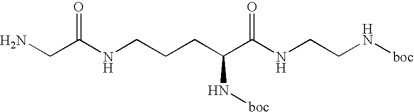

1.03 g (1.82 mmol) of the compound from Example 88A are dissolved in 60 ml of ethanol, and 100 mg (0.09 mmol) of Pd/C (10%) are added. The mixture is hydrogenated under atmospheric pressure overnight, and filtered through celite, and the filtrate is concentrated in vacuo. The solid obtained in this way is reacted further without purification.

Yield: 693 mg (84% of theory)

LC-MS (Method 3): Rₜ=1.41 min.

MS (ESI): m/z=432 (M+H)⁺

### Example 90A

Benzyl tert-butyl-[5-({(2S)-2,5-bis[(tert-butoxycarbonyl)amino]pentyl}amino)-5-oxopentane-1,3-diyl]biscarbamate

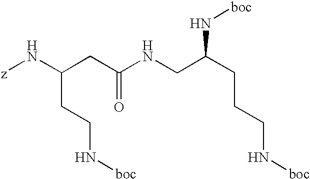

0.146 g (0.40 mmol) of 3-{[(benzyloxy)carbonyl]amino}-5-[(tert-butoxycarbonyl)amino]pentanoic acid (Bioorg. Med. Chem. 2003, 13, 241-246) and 0.164 g (0.52 mmol) of tert-butyl {(4S)-5-amino-4-[(tert-butoxycarbonyl)amino]pentyl}carbamate (Example 53A) are dissolved in 8 ml of dimethylformamide under argon. Then, at 0° C. (ice bath), 0.10 g (0.52 mmol) of EDC and 0.009 g (0.12 mmol) of HOBt are added. The mixture is slowly warmed to RT and stirred at RT for 12 h. The solution is concentrated in vacuo and the residue is taken up in ethyl acetate. The organic phase is washed successively with saturated sodium bicarbonate and sodium chloride solutions, dried over magnesium sulfate and concentrated in vacuo. The remaining solid is dried to constant weight under high vacuum.

Yield: 0.232 g, (87% of theory)

LC-MS (Method 3): Rₜ=2.73 min

MS (ESI): m/z=666 (M+H)⁺

### Example 91A

### tert-Butyl [3-amino-5-({(2S)-2,5-bis[(tert-butoxycarbonyl)amino]pentyl}amino)-5-oxopentyl]carbamate

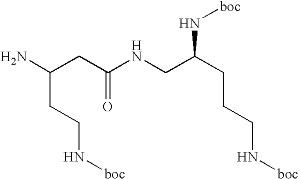

35 mg of palladium on activated carbon (10%) are added to a mixture of 0.232 g (0.35 mmol) of the compound from Example 90A in 10 ml of ethanol, and the mixture is then hydrogenated under atmospheric pressure for 12 h. The reaction mixture is filtered through kieselguhr, and the filtrate is concentrated in vacuo and dried under high vacuum. The crude product is reacted without further purification.

Yield: 0.175 g (94% of theory)

LC-MS (Method 3): Rₜ=1.8 min

MS (ESI): m/z=532 (M+H)⁺

Examples 92A and 93A listed in the following table are prepared from the corresponding starting compounds in analogy to the procedure for Example 50A detailed above:

| Ex. No. | Structure | Prepared from | Analytical Data |
|---|---|---|---|
| 92A | 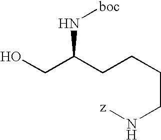 | N⁶-[(Benzyloxy)-carbonyl]-N²-(tert-butoxycarbonyl)-L-lysine | LC-MS (Method 2): Rₜ = 1.94 minMS (ESI): m/z = 367 (M + H)⁺ |
| 93A | 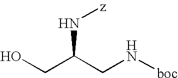 | N-[(Benzyloxy)-carbonyl]-3-[(tert-butoxycarbonyl)-amino]-L-alanine | LC-MS (Method 1): Rₜ = 1.98 minMS (ESI): m/z =325 (M + H)⁺ |

### Example 94A

### Benzyl [(1S)-2-amino-1-(hydroxymethyl)ethyl]carbamate hydrochloride

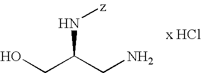

A mixture of 269 mg (0.83 mmol) of benzyl tert-butyl [(2S)-3-hydroxypropane-1,2-diyl]biscarbamate (Example 93A) and 5 ml of a 4M hydrogen chloride-dioxane solution is stirred at RT for 2 h. The reaction solution is concentrated, coevaporated several times with dichloromethane and dried under high vacuum. The crude product is reacted without further purification.

Yield: 212 mg (98% of theory)

LC-MS (Method 2): Rₜ=0.55 min

MS (ESI): m/z=225 (M−HCl+H)⁺.

Examples 95A to 102A listed in the following table are prepared from the corresponding starting materials in analogy to the procedure of Example 48A detailed above:

| Ex. No. | Structure | Prepared from | Analytical Data |
|---|---|---|---|
| 95A | 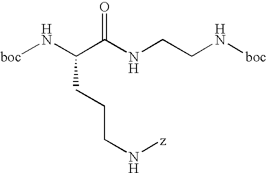 | N⁵-[(Benzyloxy)-carbonyl]-N²-(tert-butoxycarbonyl)-L-ornithineand tert-butyl-(2-aminoethyl)carbamate | LC-MS (Method 1):Rₜ = 2.33 minMS (ESI): m/z =509 (M + H)⁺ |
| 96A | 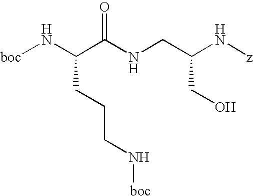 | N²,N⁵-Bis(tert-butoxycarbonyl)-L-ornithineand Ex. 94A | LC-MS (Method 1):Rₜ= 2.20 minMS (ESI): m/z =539 (M + H)⁺ |
| 97A | 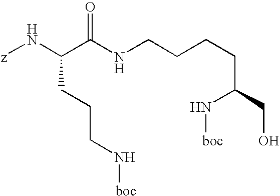 | N²-[(Benzyloxy)-carbonyl]-N⁵-(tert-butoxycarbonyl)-L-ornithineand Ex. 103A | LC-MS (Method 1):Rₜ = 2.31 minMS (ESI): m/z =581 (M + H)⁺ |
| 98A | 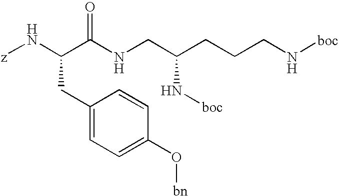 | O-Benzyl-N-[(benzyloxy)carbonyl]-L-tyrosineand Ex. 53A | LC-MS (Method 2):Rₜ = 2.79 minMS (ESI): m/z =705 (M + H)⁺ |
| 99A | 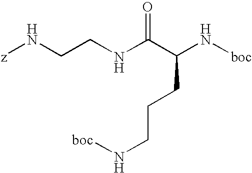 | N²,N⁵-Bis(tert-butoxycarbonyl)-L-ornithineand benzyl-(2-aminoethyl)carbamate | LC-MS (Method 2):Rₜ 2.15 minMS (ESI): m/z =509 (M + H)⁺ |
| 100A | 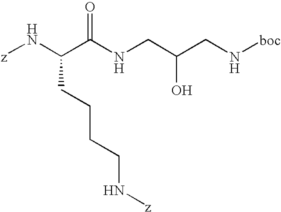 | N⁶-[(Benzyloxy)-carbonyl]-N²-(tert-butoxycarbonyl)-L-lysineand tert-butyl (3-amino-2-hydroxypropyl)carbamate | LC-MS (Method 3):Rₜ = 2.4 minMS (ESI): m/z =553 (M + H)⁺ |
| 101A | 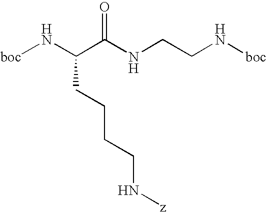 | N⁶-[(Benzyloxy)-carbonyl]-N²-(tert-butoxycarbonyl)-L-lysineand benzyl (2-aminoethyl)carbamate | LC-MS (Method 3):Rₜ = 2.49 minMS (ESI): m/z =523 (M + H)⁺ |
| 102A | 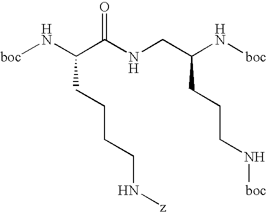 | N⁶-[(Benzyloxy)-carbonyl]-N²-(tert-butoxycarbonyl)-L-lysineand Ex. 53A | LC-MS (Method 2):Rₜ = 2.55 minMS (ESI): m/z =680 (M + H)⁺ |

Examples 103A to 111A listed in the following table are prepared from the corresponding starting materials in analogy to the procedure of Example 49A detailed above:

| | | Prepared from | |
|---|---|---|---|
| Ex. No. | Structure | Example | Analytical Data |
| 103A | 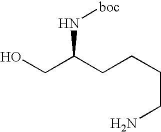 | 92A | MS (ESI): m/z = 233 (M + H)⁺ |
| 104A | 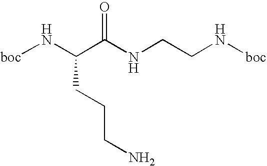 | 95A | MS (ESI): m/z = 375 (M + H)⁺ |
| 105A | 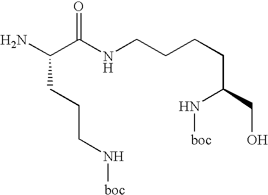 | 97A | MS (ESI): m/z = 447 (M + H)⁺ |
| 106A | 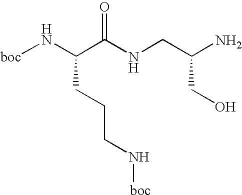 | 96A | MS (ESI): m/z = 405 (M + H)⁺ |
| 107A | 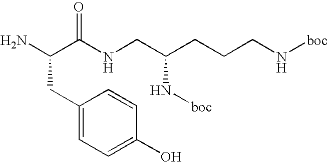 | 98A | LC-MS (Method 3): Rₜ = 1.67 minMS (ESI): m/z = 481 (M + H)⁺ |
| 108A | 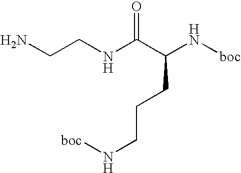 | 99A | MS (ESI): m/z = 375 (M + H)⁺ |
| 109A | 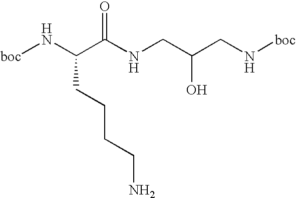 | 100A | MS (ESI): m/z = 419 (M + H)⁺ |
| 110A | 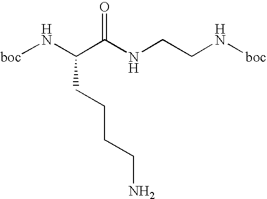 | 101A | MS (ESI): m/z = 388 (M + H)⁺ |
| 111A | 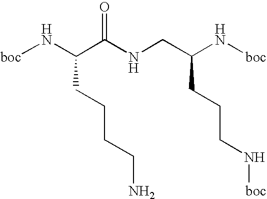 | 102A | MS (ESI): m/z = 546 (M + H)⁺ |

### Example 112A

### tert-Butyl (2-{[(2S)-2-[(tert-butoxycarbonyl)amino]-5-({[(8S,11S,14S)-14-[(tert-butoxycarbonyl)amino]-11-{3-[(tert-butoxycarbonyl)amino]propyl}-17-hydroxy-9-methyl-10,13-dioxo-9,12-diazatricyclo[14.3.1.1^{2,6}]henicosa-1(20),2(21),3,5,16,18-hexaen-8-yl]carbonyl}amino)pentanoyl]amino}ethyl)carbamate

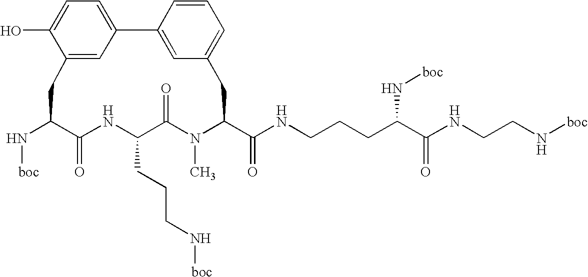

50 mg (0.05 mmol) of (8S,11S,14S)-14-[(tert-butoxycarbonyl)amino]-11-{3-[(tert-butoxycarbonyl)amino]propyl}-17-hydroxy-9-methyl-10,13-dioxo-9,12-diazatricyclo[14.3.1.1^{2,6}]-henicosa-1(20),2(21),3,5,16,18-hexaene-8-carboxylic acid (Example 46A) and 34 mg (0.09 mmol) of N²-(tert-butoxycarbonyl)-N-{2-[(tert-butoxycarbonyl)amino]ethyl}-L-ornithinamide (Example 104A) are dissolved in 2.5 ml of DMF and cooled to 0° C. 15 mg (0.08 mmol) of EDC and 6 mg (0.05 mmol) of HOBt are added and the mixture is stirred at room temperature for 12 h. The reaction mixture is concentrated on a rotary evaporator in vacuo. The crude product is reacted without further purification.

Yield: 215 mg (88% of theory)

LC-MS (Method 3): Rₜ=2.70 min

MS (ESI): m/z=1011 (M+H)⁺

### Example 113A

### tert-Butyl [(4S)-5-({(2S)-2,5-bis[(tert-butoxycarbonyl)amino]pentyl}amino)-4-({[(8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1(20),2(21),3,5,16,18-hexaen-8-yl]carbonyl}amino)-5-oxopentyl]carbamate

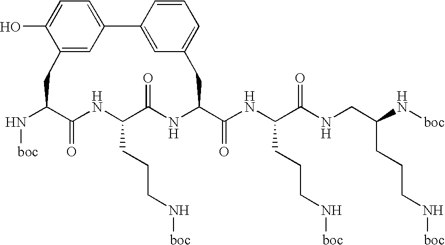

29 mg (0.05 mmol) of (8S,11S,14S)-14-[(tert-butoxycarbonyl)amino]-11-{3-[(tert-butoxycarbonyl)amino]propyl}-17-hydroxy-10,13-dioxo-9,12-diazatricyclo[14.3.1.1^{2,6}]henicosa-1 (20),2(21),3,5,16,18-hexaene-8-carboxylic acid (Example 44A) and 24 mg (0.05 mmol) of tert-butyl [(4S)-4-amino-5-({(2S)-2,5-bis[(tert-butoxycarbonyl)amino]pentyl}amino)-5-oxopentyl]carbamate (Example 57A) are dissolved in 2.0 ml DMF and cooled to 0° C. 15 mg (0.08 mmol) of EDC and 6 mg (0.05 mmol) of HOBt are added and the mixture is stirred at room temperature for 12 h. The reaction mixture is concentrated on a rotary evaporator in vacuo and purified by chromatography over Sephadex-LH20 (mobile phase: methanol/acetic acid 0.25%).

Yield: 53 mg (54% of theory)

LC-MS (Method 2): Rₜ=2.68 min

MS (ESI): m/z=1154 (M+H)⁺

### Example 114A

### tert-Butyl (2-{[(3S)-3-[(tert-butoxycarbonyl)amino]-7-({[(8S,11S,14S)-14-[(tert-butoxycarbonyl)amino]-11-{3-[(tert-butoxycarbonyl)amino]propyl}-17-hydroxy-9-methyl-10,13-dioxo-9,12-diazatricyclo[14.3.1.1^{2,6}]henicosa-1(20),2(21),3,5,16,18-hexaen-8-yl]carbonyl}amino)heptanoyl]amino}ethyl)carbamate

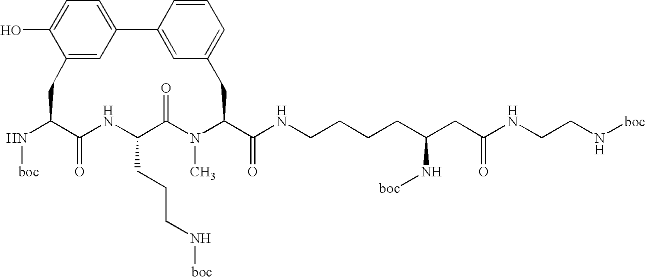

40 mg (0.06 mmol) of (8S,1S,14S)-14-[(tert-butoxycarbonyl)amino]-11-{3-[(tert-butoxycarbonyl)amino]propyl}-17-hydroxy-9-methyl-10,13-dioxo-9,12-diazatricyclo[14.3.1.1^{2,6}]henicosa-1 (20),2(21),3,5,16,18-hexaene-8-carboxylic acid (Example 46A) and 46 mg (0.08 mmol) of tert-butyl {(1S)-5-amino-1-[2-({2-[(tert-butoxycarbonyl)amino]ethyl}amino)-2-oxoethyl]pentyl}carbamate (Example 65A) are dissolved in 2.0 ml of DMF and cooled to 0° C. 15 mg (0.08 mmol) of EDC, 3 mg (0.02 mmol) of HOBt and 0.01 ml (0.08 mmol) of triethylamine are added and the mixture is stirred at room temperature for 12 h. The reaction mixture is concentrated on a rotary evaporator in vacuo and purified by a preparative HPLC.

Yield: 6 mg (9% of theory)

LC-MS (Method 2): Rₜ=2.47 min

MS (ESI): m/z=1039 (M+H)⁺

### Example 115A

### Benzyl ((1S)-4-{[(2S)-5-{[(benzyloxy)carbonyl]amino}-2-({[(8S,11S,14S)-14-[(tert-butoxycarbonyl)amino]-11-{3-[(tert-butoxycarbonyl)amino]propyl}-17-hydroxy-9-methyl-10,13-dioxo-9,12-diazatricyclo[14.3.1.1^{2,6}]henicosa-1(20),2(21),3,5,16,18-hexaen-8-yl]carbonyl}amino)pentanoyl]amino}-1-{2-[(2-{[(benzyloxy)carbonyl]amino}ethyl)amino]-2-oxoethyl}butyl)carbamate

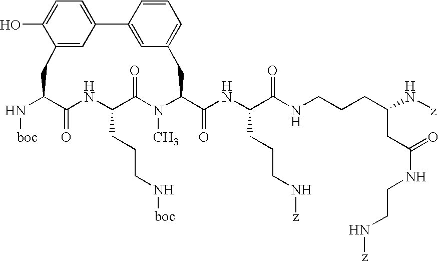

65 mg (0.06 mmol) of (8S,1S,14S)-14-[(tert-butoxycarbonyl)amino]-1′-({3-[(tert-butoxycarbonyl)amino]propyl}-17-hydroxy-9-methyl-10,13-dioxo-9,12-diazatricyclo[14.3.1.1^{2,6}]-henicosa-1(20),2(21),3,5,16,18-hexaene-8-carboxylic acid (Example 46A) and 120 mg (0.13 mmol) of benzyl ((5S,11S)-5-amino-11-{[(benzyloxy)carbonyl]amino}-6,13,18-trioxo-20-phenyl-1 g-oxa-7,14,17-triazaicos-1-yl)carbamate hydrochloride (Example 63A) are dissolved in 3.0 ml of DMF and cooled to 0° C. 25 mg (0.13 mmol) of EDC, 4 mg (0.03 mmol) of HOBt and 0.02 ml (0.13 mmol) of triethylamine are added and the mixture is stirred at room temperature for 12 h. The reaction mixture is concentrated on a rotary evaporator in vacuo and purified by preparative HPLC.

Yield: 50 mg (25% of theory).

LC-MS (Method 3): Rₜ=2.92 min

MS (ESI): m/z=1341 (M+H)⁺

### Example 116A

### tert-Butyl {3-[(8S,11S,14S)-8-[({(1S)-4-amino-1-[({(4S)-4-amino-6-[(2-aminoethyl)amino]-6-oxohexyl}amino)carbonyl]butyl}amino)carbonyl]-14-[(tert-butoxycarbonyl)amino]-17-hydroxy-9-methyl-10,13-dioxo-9,12-diazatricyclo[14.3.1.1^{2,6}]henicosa-1(20),2(21), 3,5,16,18-hexaen-11-yl]propyl}carbamate tris(hydrotrifluoracetate)

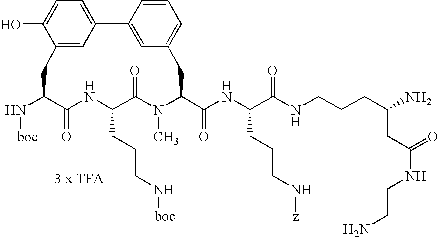

49 mg (0.04 mmol) of benzyl ((1S)-4-{[(2S)-5-{[(benzyloxy)carbonyl]amino}-2-({[(8S,11S,14S)-14-[(tert-butoxycarbonyl)amino]-11-{3-[(tert-butoxycarbonyl)amino]propyl}-17-hydroxy-9-methyl-10,13-dioxo-9,12-diazatricyclo[14.3.1.1^{2,6}]henicosa-1(20), 2(21),3,5,16,18-hexaen-8-yl]carbonyl}amino)pentanoyl]amino}-1-{2-[(2-{[(benzyloxy)carbonyl]amino}ethyl)amino]-2-oxoethyl}butyl)carbamate (Example 115A) are dissolved in 10 ml of glacial acetic acid/water (4:1), 5 mg of Pd/C (10%) are added and the mixture hydrogenated under atmospheric pressure and a hydrogen atmosphere for 12 h. Suction filtration is carried out, and the reaction mixture is concentrated in vacuo and purified by preparative HPLC (Kromasil 100 C18, 5 μm 250 mm×20 mm; mobile phase acetonitrile/0.2% aqueous trifluoroacetic acid 5:95→95:5).

Yield: 9 mg (19% of theory)

LC-MS (Method 3): Rₜ=1.45 min

MS (ESI): m/z=939 (M+H)⁺

### Example 117A

### tert-Butyl (2-{[(2S)-2-[(tert-butoxycarbonyl)amino]-5-({[(8S,11S,14S)-14-[(tert-butoxycarbonyl)amino]-11-{(2R)-3-[(tert-butoxycarbonyl)amino]-2-hydroxypropyl}-17-hydroxy-10,13-dioxo-9,12-diazatricyclo[14.3.1.1^{2,6}]henicosa-1 (20),2(21),3,5,16,18-hexaen-8-yl]carbonyl}amino)pentanoyl]amino}ethyl)carbamate

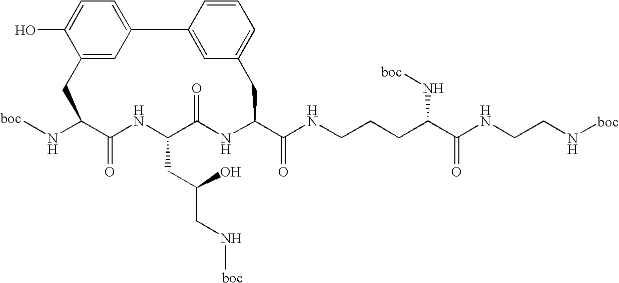

Under argon, 50 mg (0.076 mmol) of the compound from Example 43A and 37 mg (0.1 mmol) of N²-(tert-butoxycarbonyl)-N-{2-[(tert-butoxycarbonyl)amino]ethyl}-L-ornithinamide (Example 104A) are dissolved in 2 ml of dimethylformamide. Then, at 0° C. (ice bath), 19 mg (0.1 mmol) of EDC and 3.1 mg (0.023 mmol) of HOBt are added. The mixture is slowly warmed to RT and stirred at RT for 12 h. The solution is concentrated in vacuo and the residue is stirred with water. The remaining solid is collected by suction filtration and purified via preparative HPLC.

Yield: 6 mg (7% of theory)

LC-MS (Method 3): Rₜ=2.49 min

MS (ESI): m/z=1013 (M+H)⁺

### Example 118A

### Di-tert-butyl (5-{[(3S)-6-[(tert-butoxycarbonyl)amino]-3-({[(8S,11S,14S)-14-[(tert-butoxycarbonyl)amino]-11-{(2R)-3-[(tert-butoxycarbonyl)amino]-2-hydroxypropyl}17-hydroxy-9-methyl-10,13-dioxo-9,12-diazatricyclo[14.3.1.1^{2,6}]henicosa-1(20),2(21),3,5,16,18-hexaen-8-yl]carbonyl}amino)hexanoyl]amino}pentane-1,4-diyl)biscarbamate

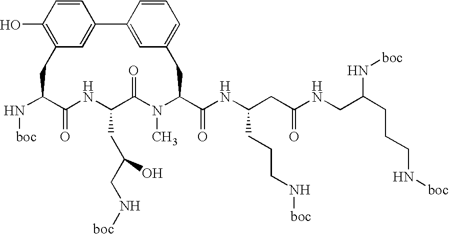

30.7 mg (0.046 mmol) of (8S,11S,14S)-14-[(tert-butoxycarbonyl)amino]-11-{(2R)-3-[(tert-butoxycarbonyl)amino]-2-hydroxypropyl}-17-hydroxy-9-methyl-10,13-dioxo-9,12-diazatricyclo[14.3.1.1^{2,6}]henicosa-1(20),2(21),3,5,16,18-hexaene-8-carboxylic acid (Example 45A) and 30 mg (0.055 mmol) of the compound from Example 81A are dissolved in 2.0 ml of DMF and cooled to 0° C. 11.4 mg (0.06 mmol) of EDC and 2 mg (0.015 mmol) of HOBt are added and the mixture is stirred at room temperature for 12 h. The reaction mixture is concentrated on a rotary evaporator in vacuo and purified by chromatography over Sephadex-LH20 (mobile phase: methanol/acetic acid 0.25%).

Yield: 13 mg (24% of theory)

LC-MS (Method 3): Rₜ=2.84 min

MS (ESI): m/z=1198 (M+H)⁺

Example 119A listed in the following table is prepared in analogy to the procedure of Example 112A.

| Example | Precursor | | |
|---|---|---|---|
| No. | Example | Structure | Analytical Data |
| 119A | 108A + 44A | 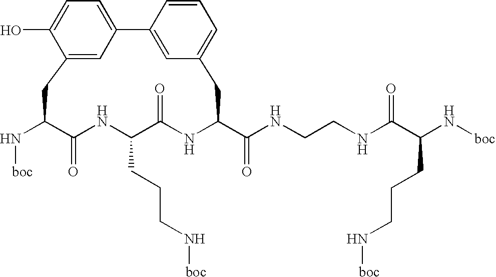 | LC-MS (Method 3): Rₜ = 2.57 minMS (ESI): m/z = 997 (M + H)⁺. |

Examples 120A to 126A listed in the following table are prepared in analogy to the procedure of Example 117A.

| Exam- | | | |
|---|---|---|---|
| ple | Precursor | | |
| No. | Example | Structure | Analytical Data |
| 120A | 49A + 43A | 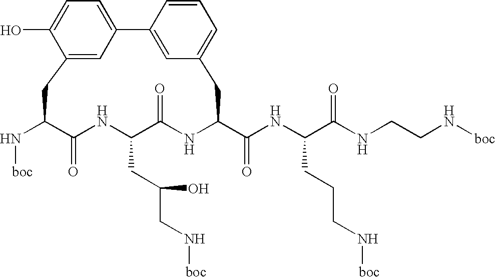 | LC-MS (Method 3): Rₜ = 2.57minMS (ESI): m/z = 1013(M + H)⁺. |
| 121A | 55A + 43A | 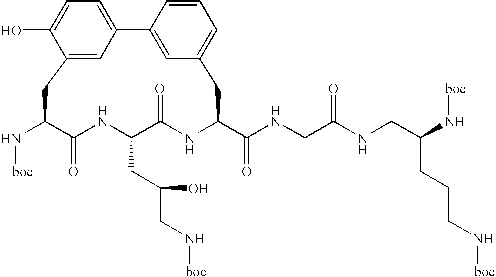 | LC-MS (Method 1): Rₜ = 2.5min.MS (ESI): m/z = 1013(M + H)⁺. |
| 122A | 106A + 43A | 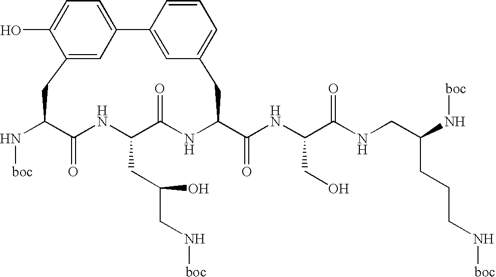 | LC-MS (Method 3): Rₜ = 2.46min.MS (ESI): m/z = 1043(M + H)⁺. |
| 123A | 85A + 46A | 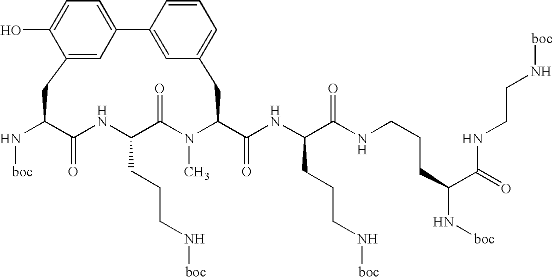 | LC-MS (Method 1): Rₜ 2.71minMS (ESI): m/z = 1225(M + H)⁺. |
| 124A | 89A + 46A | 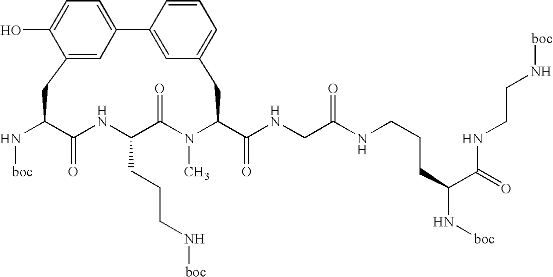 | LC-MS (Method 1): Rₜ = 2.46minMS (ESI): m/z = 1069(M + H)⁺. |
| 125A | 49A + 46A | 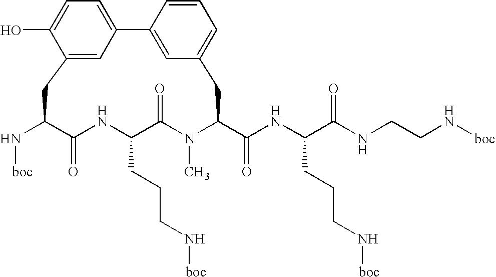 | LC-MS (Method 3): Rₜ = 2.74minMS (ESI): m/z = 1011(M + H)⁺. |
| 126A | 87A + 46A | 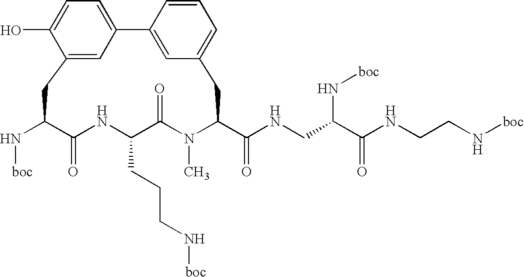 | LC-MS (Method 2): Rₜ = 2.47minMS (ESI): m/z 983 (M + H)⁺. |

Examples 127A to 149A listed in the following table are prepared in analogy to the procedure of Example 113A.

| Ex- | Pre- | | |
|---|---|---|---|
| am- | cursor | | |
| ple | Exam- | | |
| No. | ple | Structure | Analytical Data |
| 127A | 59A +44A | 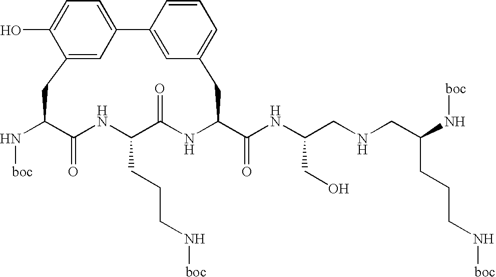 | LC-MS (Method 3): Rₜ =2.59 minMS (ESI): m/z =1027 (M + H)⁺. |
| 128A | 105A +44A | 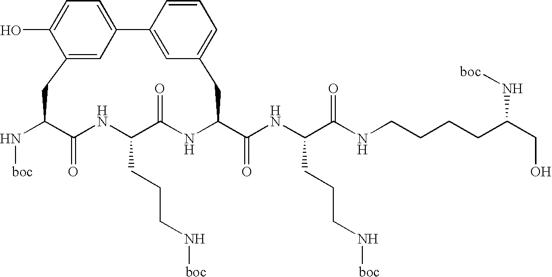 | LC-MS (Method 3): Rₜ =2.65 minMS (ESI): m/z =1069 (M + H)⁺. |
| 129A | 67A +44A | 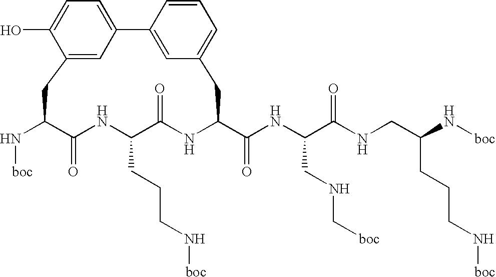 | LC-MS (Method 3): Rₜ =2.82 minMS (ESI): m/z =1126 (M + H)⁺. |
| 130A | 49A +44A | 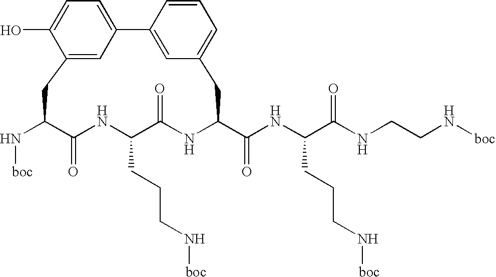 | LC-MS (Method 2): Rₜ =2.41 minMS (ESI): m/z =997 (M + H)⁺. |
| 131A | 55A +44A | 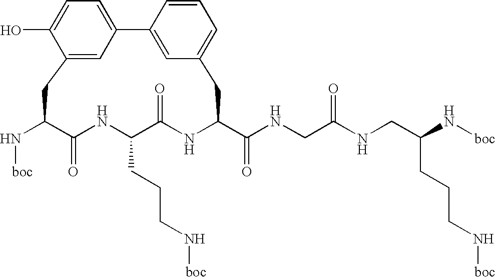 | LC-MS (Method 1): Rₜ =2.56 minMS (ESI): m/z =997 (M + H)⁺. |
| 132A | 107A +44A | 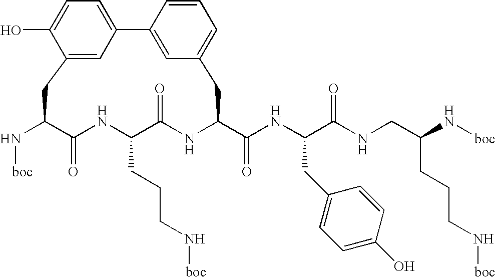 | LC-MS (Method 3): Rₜ =2.67 minMS (ESI): m/z =1103 (M + H)⁺. |
| 133A | 71A +44A | 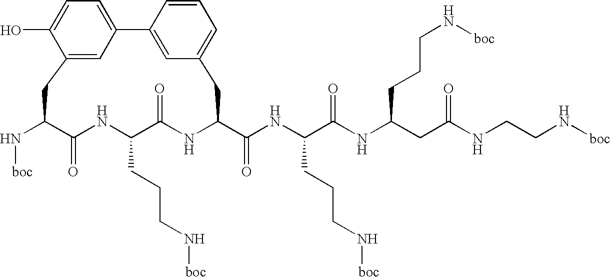 | LC-MS (Method 2): Rₜ =2.56 minMS (ESI): m/z =1225 (M + H)⁺. |
| 134A | 71A +43A | 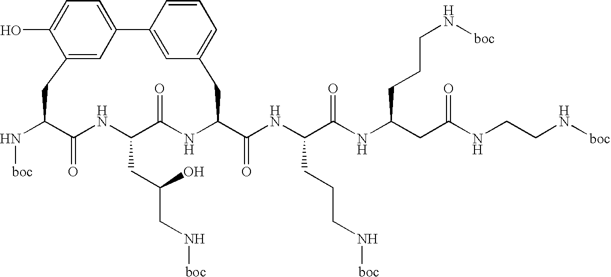 | LC-MS (Method 1): Rₜ =2.64 minMS (ESI): m/z =1241 (M + H)⁺. |
| 135A | 75A +43A | 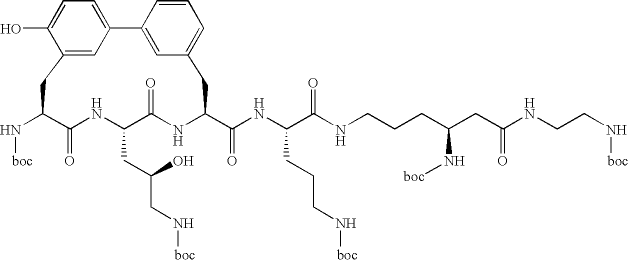 | LC-MS (Method 2): Rₜ =2.47 minMS (ESI): m/z =1241 (M + H)⁺. |
| 136A | 75A +44A | 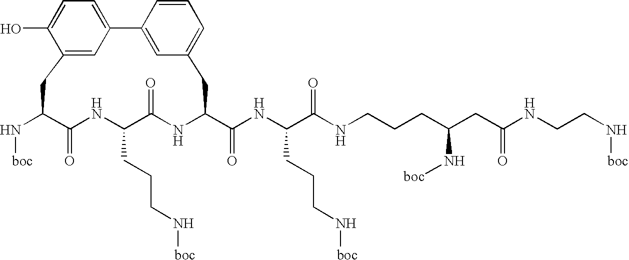 | LC-MS (Method 2): Rₜ =2.52 minMS (ESI): m/z =1225 (M + H)⁺. |
| 137A | 57A +43A | 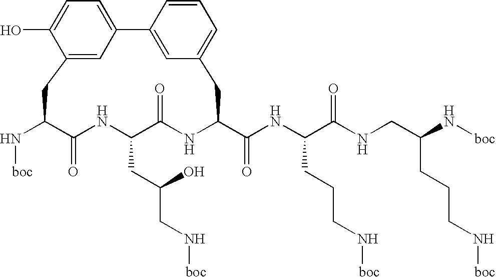 | LC-MS (Method 3): Rₜ =2.87 minMS (ESI): m/z =1170 (M + H)⁺. |
| 138A | 79A +43A | 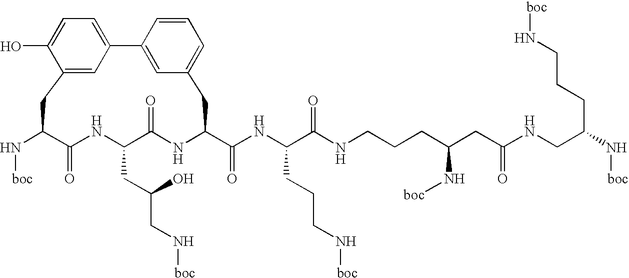 | LC-MS (Method 3): Rₜ =2.92 minMS (ESI): m/z =1398 (M + H)⁺. |
| 139A | 79A +44A | 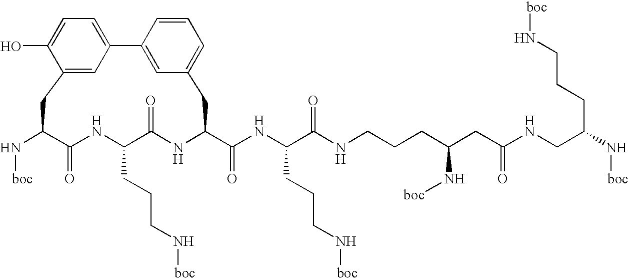 | LC-MS (Method 2): Rₜ =2.74 minMS (ESI): m/z =1382 (M + H)⁺. |
| 140A | 83A +44A | 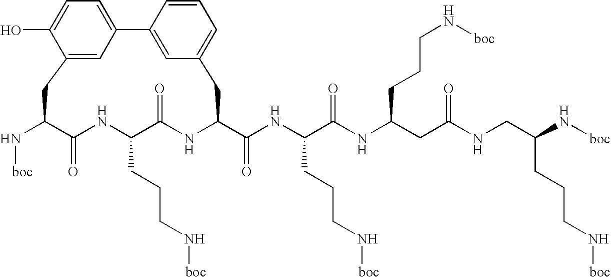 | LC-MS (Method 3): Rₜ =2.95 minMS (ESI): m/z =1382 (M + H)⁺. |
| 141A | 83A +43A | 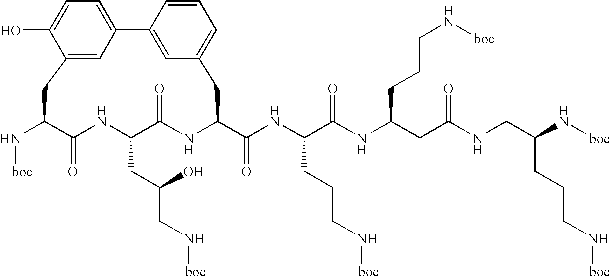 | LC-MS (Method 2): Rₜ =2.72 minMS (ESI): m/z =1398 (M + H)⁺. |
| 142A | 85A +44A | 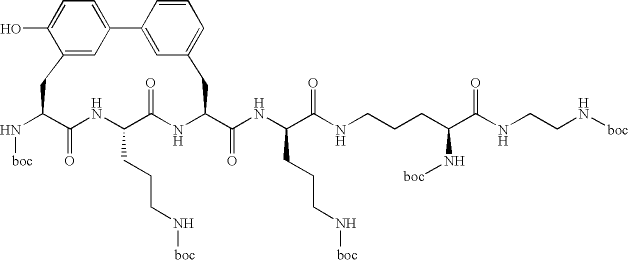 | LC-MS (Method 1): Rₜ =2.66 minMS (ESI): m/z =1211 (M + H)⁺. |
| 143A | 81A +44A | 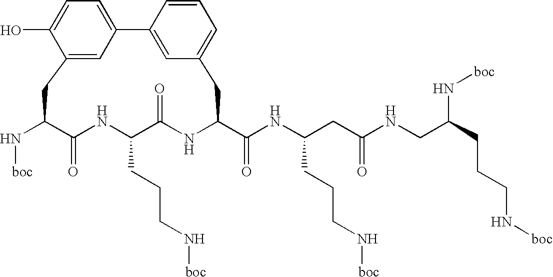 | LC-MS (Method 3): R^{t} =2.82 minMS (ESI): m/z =1168 (M + H)⁺. |
| 144A | 91A +44A | 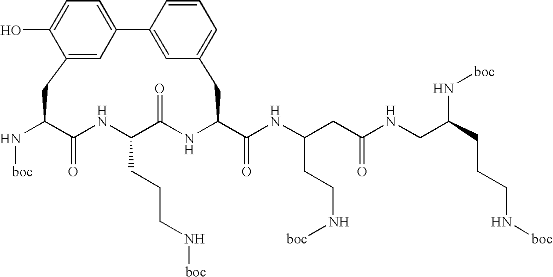 | LC-MS (Method 2): Rₜ =2.65 minMS (ESI): m/z =1154 (M + H)⁺. |
| 145A | 109A +44A | 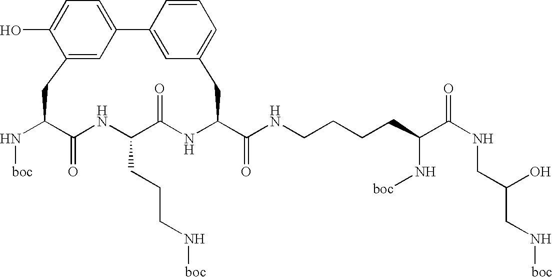 | LC-MS (Method 2): Rₜ =2.3 minMS (ESI): m/z =1041 (M + H)⁺. |
| 146A | 110A +44A | 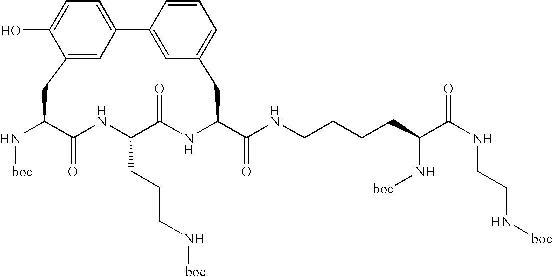 | LC-MS (Method 2): Rₜ =2.38 minMS (ESI): m/z =1011 (M + H)⁺. |
| 147A | 111A +44A | 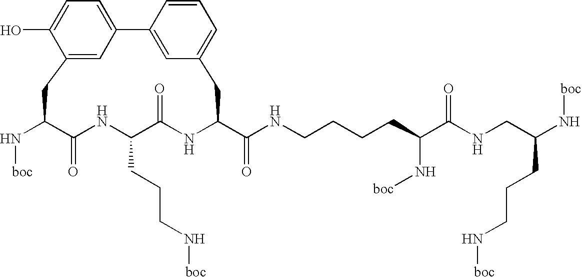 | LC-MS (Method 2): Rₜ =2.62 minMS (ESI): m/z =1168 (M + H)⁺. |
| 148A | 67A +45A | 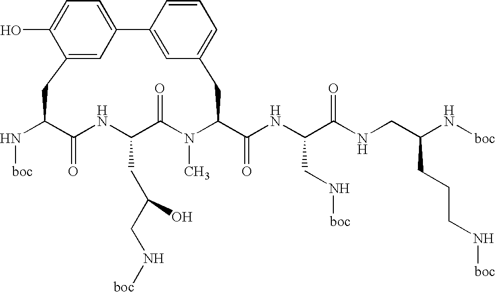 | LC-MS (Method 3): Rₜ =2.88 minMS (ESI): m/z =1156 (M + H)⁺. |
| 149A | 49A +45A | 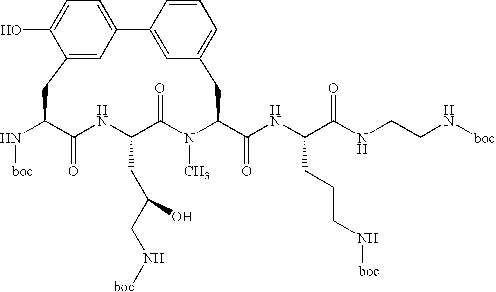 | LC-MS (Method 3): Rₜ =2.64 minMS (ESI): m/z =1027 (M + H)⁺. |

Examples 150A to 187A listed in the following table are prepared from the appropriate starting materials in analogy to the procedure of Example 48A.

| Ex. No. | Structure | Prepared from | Analytical Data |
|---|---|---|---|
| 150A | 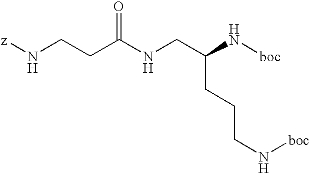 | N-[(Benzyloxy)-carbonyl]-beta-alanineand Ex. 53A | LC-MS (Method 1): Rₜ = 2.19minMS (ESI): m/z = 523(M + H)⁺ |
| 151A | 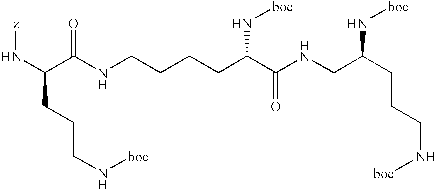 | N²-[(Benzyloxy)-carbonyl]-N⁵-(tert-butoxycarbonyl)-D-ornithineand Ex. 111A | LC-MS (Method 2): Rₜ = 2.62minMS (ESI): m/z = 894(M + H)⁺ |
| 152A | 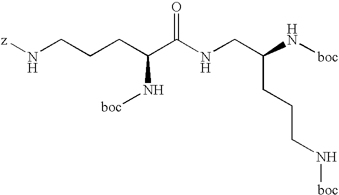 | N⁵-[(Benzyloxy)-carbonyl]-N²-(tert-butoxycarbonyl)-L-ornithineand Ex. 53A | LC-MS (Method 3): Rₜ = 2.68minMS (ESI): m/z = 666(M + H)⁺ |
| 153A | 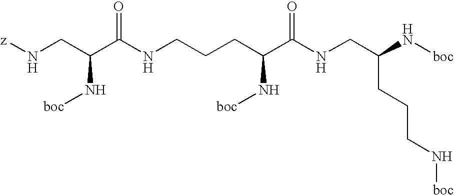 | 3-{[(Benzyloxy)-carbonyl]amino}-N-(tert-butoxycarbonyl)-L-alanineand Ex. 190A | LC-MS (Method 3): Rₜ = 2.76minMS (ESI): m/z = 852(M + H)⁺ |
| 154A | 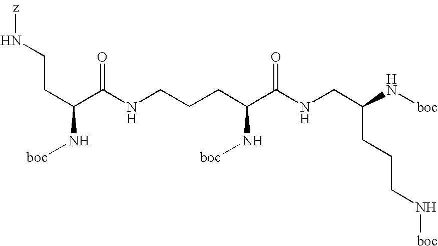 | (2S)-4-{[(Benzyloxy)-carbonyl]amino}-2-[(tert-butoxycarbonyl)-amino]butanoic acidand Ex. 190A | LC-MS (Method 3): Rₜ = 2.75minMS (ESI): m/z = 866(M + H)⁺ |
| 155A | 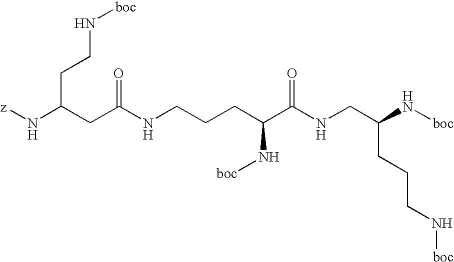 | 3-{[BenzyIoxy)-carbonyl]amino}-5-[(tert-butoxycarbonyl)-amino]pentanoic acidand Ex. 190A | LC-MS (Method 3): Rₜ = 2.85minMS (ESI): m/z = 880(M + H)⁺ |
| 156A | 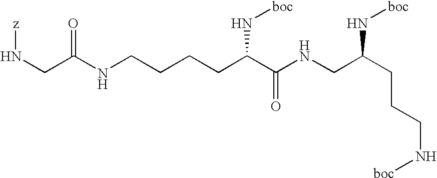 | N-[(Benzyloxy)-carbonyl]glycineand Ex. 111A | LC-MS (Method 2): Rₜ = 2.32minMS (ESI): m/z = 737(M + H)⁺ |
| 157A | 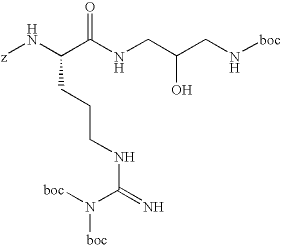 | N²-[(Benzyloxy)carbonyl]-N⁵-[[bis(tert-butoxy-carbonyl)amino]-(imino)methyl]-L-ornithineand tert-butyl(3-amino-2-hydroxy-propyl)carbamate | LC-MS (Method 2): Rₜ = 2.58minMS (ESI): m/z = 681(M + H)⁺ |
| 158A | 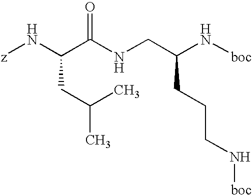 | N-[(Benzyloxy)-carbonyl]-L-leucineand Ex. 53A | LC-MS (Method 2): Rₜ = 2.53minMS (ESI): m/z = 565(M + H)⁺ |
| 159A | 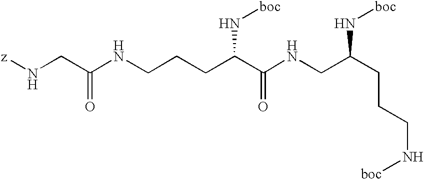 | N-[(Benzyloxy)-carbonyl]glycineand Ex. 190A | LC-MS (Method 1): Rₜ = 2.45minMS (ESI): m/z = 723(M + H)⁺ |
| 160A | 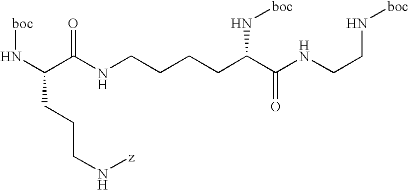 | N⁵-[(Benzyloxy)-carbonyl]-N²-(tert-butoxycarbonyl)-L-ornithine and Ex. 110A | LC-MS (Method 3): Rₜ = 2.53minMS (ESI): m/z = 737(M + H)⁺ |
| 161A | 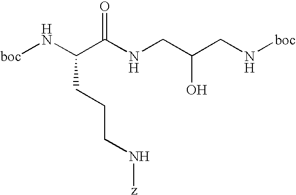 | N⁵-[(Benzyloxy)-carbonyl]-N²-(tert-butoxycarbonyl)-L-ornithineand tert-butyl (3-amino-2-hydroxy-propyl)carbamate | LC-MS (Method 3): Rₜ = 2.27minMS (ESI): m/z = 539(M + H)⁺ |
| 162A | 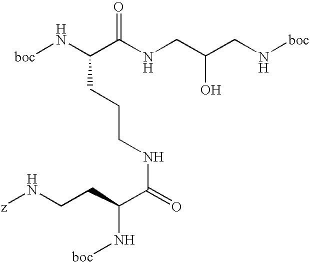 | (2S)-4-{[(Benzyloxy)-carbonyl]amino)-2-[(tert-butoxycarbonyl)amino]butanoic acidand Ex. 199A | LC-MS (Method 3): Rₜ = 2.39minMS (ESI): m/z = 739(M + H)⁺ |
| 163A | 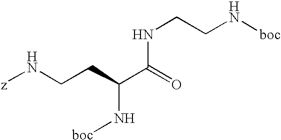 | (2S)-4-{[(Benzyloxy)-carbonyl]amino}-2-[(tert-butoxycarbonyl)amino]butanoic acidand tert-butyl(2-aminoethyl)carbamate | LC-MS (Method 3): Rₜ = 2.35minMS (ESI): m/z = 495(M + H)⁺ |
| 164A | 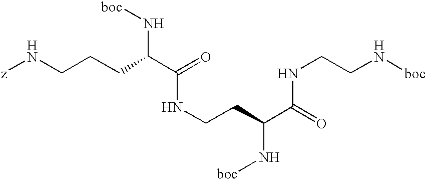 | N⁵-[(Benzyloxy)-carbonyl]-N²-(tert-butoxycarbonyl)-L-ornithineand Ex. 201A | LC-MS (Method 2): Rₜ = 2.30minMS (ESI): m/z = 709(M + H)⁺ |
| 165A | 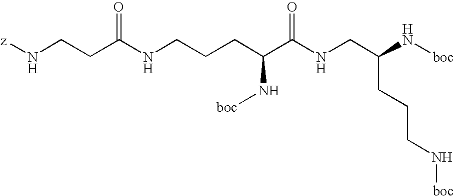 | N-[(Benzyloxy)-carbonyl]-beta-alanineand Ex. 190A | LC-MS (Method 3): Rₜ = 2.60minMS (ESI): m/z = 737(M + H)⁺ |
| 166A | 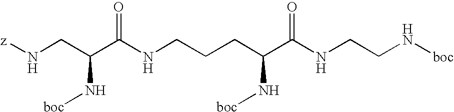 | 3-{[(Benzyloxy)-carbonyl]amino}-N-(tert-butoxycarbonyl)-L-alanineand Ex. 104A | LC-MS (Method 3): Rₜ = 2.47minMS (ESI): m/z = 695(M + H)⁺ |
| 167A | 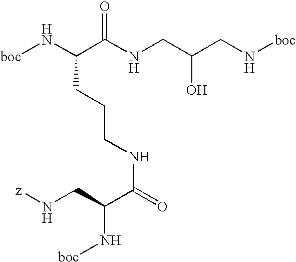 | 3-{[(Benzyloxy)-carbonyl]amino}-N-(tert-butoxycarbonyl)-L-alanineand Ex. 199A | LC-MS (Method 3): Rₜ = 2.39minMS (ESI): m/z = 725(M + H)⁺ |
| 168A | 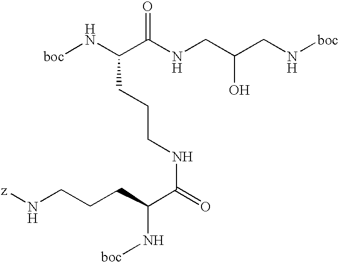 | N⁵-[(Benzyloxy)-carbonyl]-N²-(tert-butoxycarbonyl)-L-ornithineand Ex. 199A | LC-MS (Method 3): Rₜ = 2.40minMS (ESI): m/z = 753(M + H)⁺ |
| 169A | 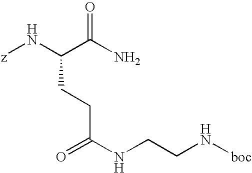 | N²(Benzyloxy)-carbonyl]-L-alpha-glutamineand tert-butyl(2-aminoethyl)carbamate | LC-MS (Method 3): Rₜ = 1.93minMS (ESI): m/z = 423(M + H)⁺ |
| 170A | 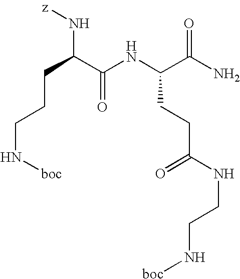 | N²-[(Benzyloxy)-carbonyl]-N⁵-(tert-butoxycarbonyl)-D-ornithineand Ex. 207A | LC-MS (Method 3): Rₜ = 2.26minMS (ESI): m/z = 637(M + H)⁺ |
| 171A | 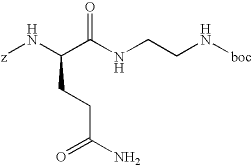 | N²-[(Benzyloxy)-carbonyl]-D-glutamine and tert-butyl(2-aminoethyl)carbamate | LC-MS (Method 3): Rₜ = 1.94minMS (ESI): m/z = 423(M + H)⁺ |
| 172A | 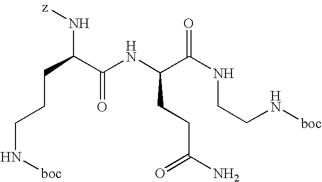 | N²-[(Benzyloxy)-carbonyl]-N⁵-(tert-butoxycarbonyl)-D-ornithineand Ex. 209A | LC-MS (Method 3): Rₜ = 2.25minMS (ESI): m/z = 637(M + H)⁺ |
| 173A | 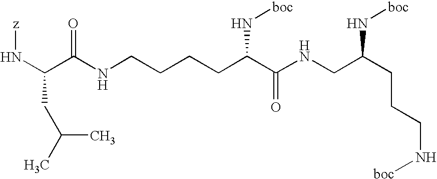 | N-[(Benzyloxy)-carbonyl]-L-leucineand Ex. 111A | LC-MS (Method 2): Rₜ = 2.82minMS (ESI): m/z = 793(M + H)⁺ |
| 174A | 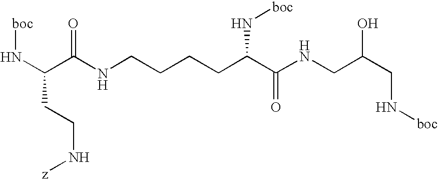 | (2S)-4-{[(Benzyloxy)-carbonyl]amino}-2-[(tert-butoxycarbonyl)-amino]butanoicacid andEx. 109A | LC-MS (Method 3): Rₜ = 2.44minMS (ESI): m/z = 753(M + H)⁺ |
| 175A | 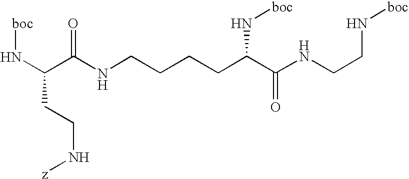 | (2S)-4-{[(Benzyloxy)-carbonyl]aminol-2-[(tert-butoxycarbonyl)-amino]butanoicacid andEx. 110A | LC-MS (Method 3): Rₜ = 2.52minMS (ESI): m/z = 723(M + H)⁺ |
| 176A | 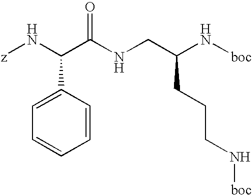 | (2S)-{[(Benzyloxy)-carbonyl]amino}-(phenyl)acetic acidand Ex. 53A | LC-MS (Method 2): Rₜ = 2.50minMS (ESI): m/z = 585(M + H)⁺ |
| 177A | 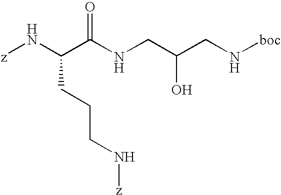 | N²,N⁵-bis-[(Benzyloxy)-carbonyl]-L-ornithineand tert-butyl(3-amino-2-hydroxy-propyl)carbamate | LC-MS (Method 2): Rₜ = 2.15minMS (ESI): m/z = 573(M + H)⁺ |
| 178A | 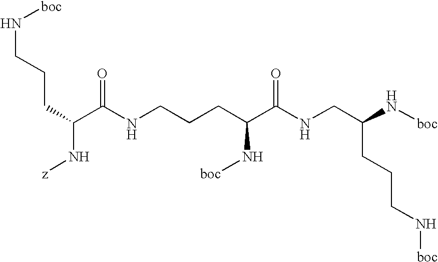 | N²-[(Benzyloxy)-carbonyl]-N⁵-(tert-butoxycarbonyl)-D-ornithineand Ex. 190A | LC-MS (Method 3): Rₜ 2.88minMS (ESI): m/z = 880(M + H)⁺ |
| 179A | 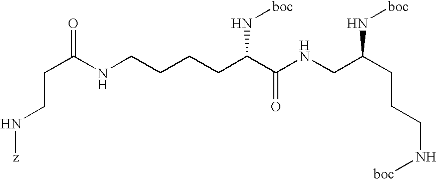 | N-[(Benzyloxy)-carbonyl]-beta-alanineand Ex. 111A | LC-MS (Method 3): Rₜ = 2.52minMS (ESI): m/z = 751(M + H)⁺ |
| 180A | 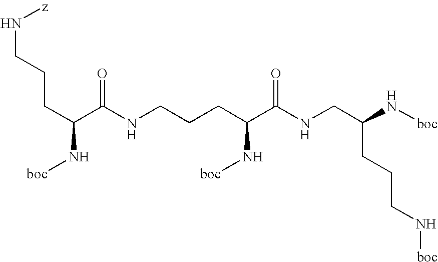 | N⁵-[(Benzyloxy)-carbonyl]-N²-(tert-butoxycarbonyl)-L-ornithineand Ex. 190A | LC-MS (Method 3): Rₜ = 2.76minMS (ESI): m/z = 880(M + H)⁺ |
| 181A | 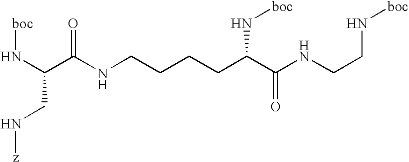 | 3-{[(Benzyloxy)-carbonyl]amino}-N-(tert-butoxycarbonyl)-L-alanineand Ex. 110A | LC-MS (Method 1): Rₜ = 2.46minMS (ESI): m/z = 709(M + H)⁺ |
| 182A | 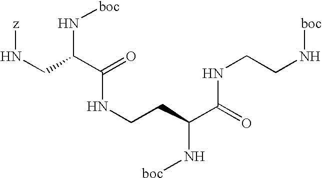 | 3-{[(Benzyloxy)-carbonyl]amino}-N-(tert-butoxycarbonyl)-L-alanineand Ex. 201A | LC-MS (Method 2): Rₜ = 2.31minMS (ESI): m/z = 681(M + H)⁺ |
| 183A | 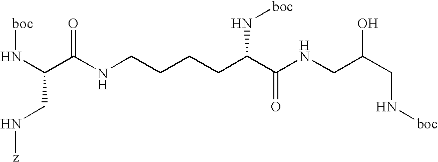 | 3-{[(Benzyloxy)-carbonyl]amino}-N-(tert-butoxycarbonyl)-L-alanineand Ex. 109A | LC-MS (Method 1): Rₜ = 2.38minMS (ESI): m/z = 739(M + H)⁺ |
| 184A | 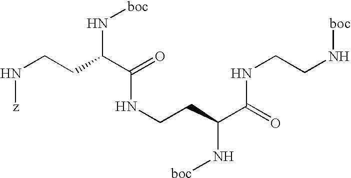 | (2S)-4-{[(Benzyloxy)-carbonyl]amino}-2-[(tert-butoxycarbonyl)amino]butanoicacid andEx. 201A | LC-MS (Method 2): Rₜ = 2.29minMS (ESI): m/z = 695(M + H)⁺ |
| 185A | 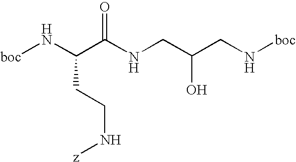 | (2S)-4-{[(Benzyloxy)-carbonyl]amino}-2-[(tert-butoxycarbonyl)amino]butanoicacid andtert-butyl (3-amino-2-hydroxypropyl)carbamate | LC-MS (Method 1): Rₜ = 2.38minMS (ESI): m/z = 525(M + H)⁺ |
| 186A | 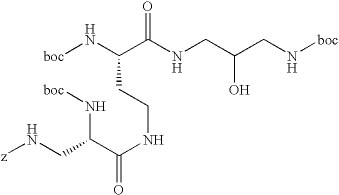 | 3-{[(Benzyloxy)-carbonyl]amino}-N-(tert-butoxycarbonyl)-L-alanineand Ex. 223A | LC-MS (Method 1): Rₜ = 2.36minMS (ESI): m/z = 711(M + H)⁺ |
| 187A | 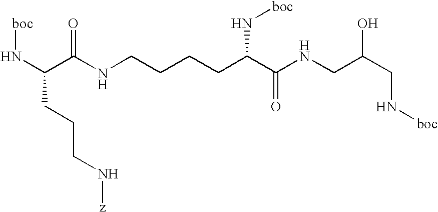 | N⁵-[(Benzyloxy)-carbonyl]-N²-(tert-butoxycarbonyl)-L-ornithineand Ex. 109A | LC-MS (Method 3): Rₜ = 2.44minMS (ESI): m/z = 767(M + H)⁺ |

Examples 188A to 224A listed in the following table are prepared from the corresponding starting materials in analogy to the procedure of Example 49A.

| Ex. No. | Structure | Prepared from | Analytical Data |
|---|---|---|---|
| 188A | 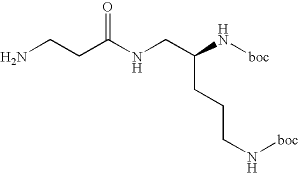 | Ex. 150A | MS (ESI): m/z = 389 (M + H)⁺ |
| 189A | 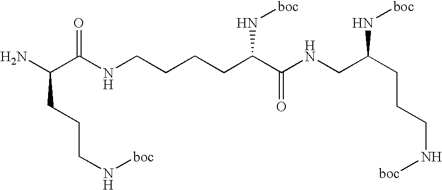 | Ex. 151A | MS (ESI): m/z = 750 (M + H)⁺ |
| 190A | 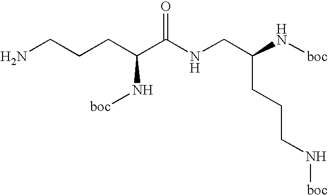 | Ex. 152A | MS (ESI): m/z = 532 (M + H)⁺ |
| 191A | 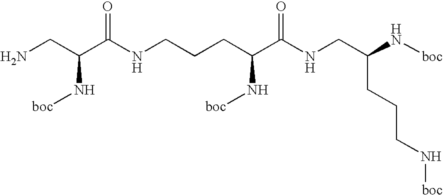 | Ex. 153A | MS (ESI): m/z = 718 (M + H)⁺ |
| 192A | 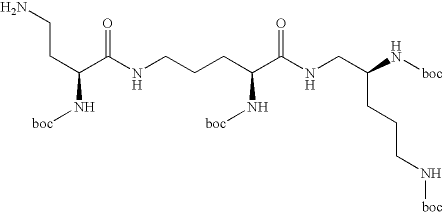 | Ex. 154A | MS (ESI): m/z = 732 (M + H)⁺ |
| 193A | 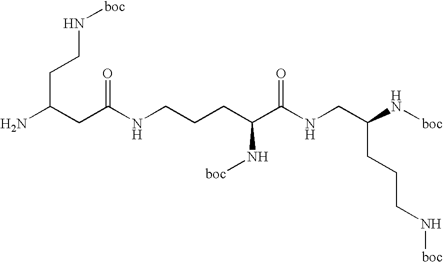 | Ex. 155A | LC-MS (Method 2): Rₜ =1.78 minMS (ESI): m/z = 746 (M + H)⁺ |
| 194A | 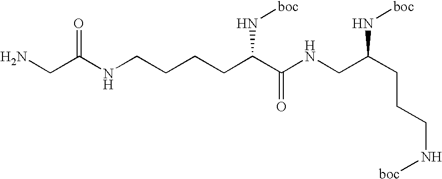 | Ex. 156A | MS (ESI): m/z = 603 (M + H)⁺ |
| 195A | 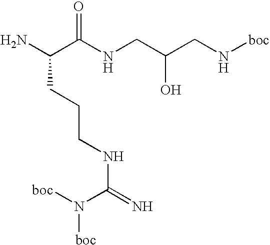 | Ex. 157A | MS (ESI): m/z = 547 (M + H)⁺ |
| 196A | 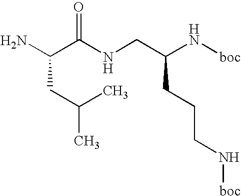 | Ex. 158A | LC-MS (Method 2): Rₜ =1.37 minMS (ESI): m/z = 431 (M + H)⁺ |
| 197A | 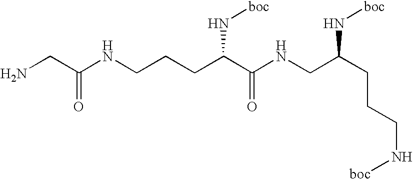 | Ex. 159A | LC-MS (Method 1): Rₜ =1.66 minMS (ESI): m/z = 589 (M + H)⁺ |
| 198A | 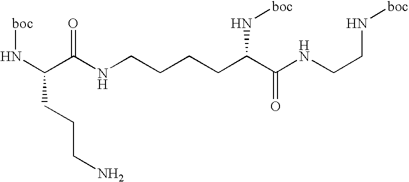 | Ex. 160A | MS (ESI): m/z = 603 (M + H)⁺ |
| 199A | 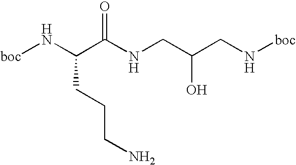 | Ex. 161A | MS (ESI): m/z = 405 (M + H)⁺ |
| 200A | 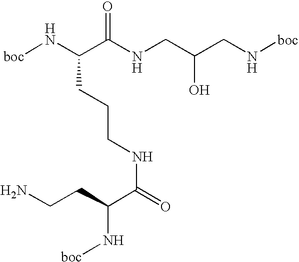 | Ex. 162A | MS (ESI); m/z = 605 (M + H)⁺ |
| 201A | 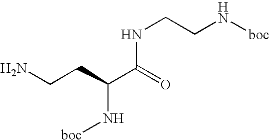 | Ex. 163A | MS (ESI): m/z = 361 (M + H)⁺ |
| 202A | 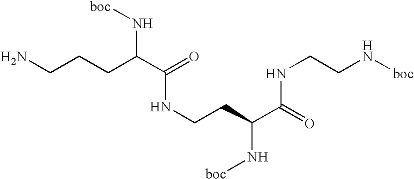 | Ex. 164A | MS (ESI): m/z = 575 (M + H)⁺ |
| 203A | 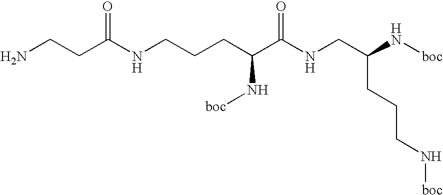 | Ex. 165A | LC-MS (Method 2): Rₜ =1.56 minMS (ESI): m/z = 603 (M + H)⁺ |
| 204A | 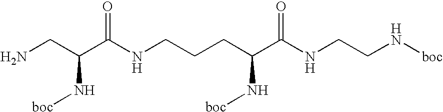 | Ex. 166A | MS (ESI): m/z = 561 (M + H)⁺ |
| 205A | 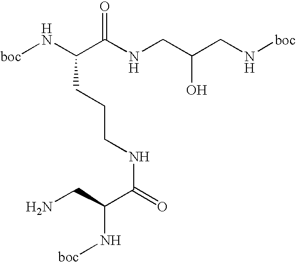 | Ex. 167A | MS (ESI): m/z = 591 (M + H)⁺ |
| 206A | 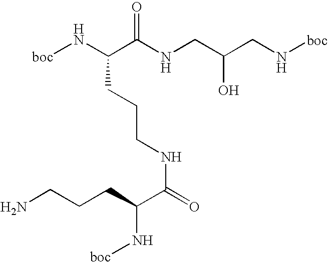 | Ex. 168A | MS (ESI): m/z = 619 (M + H)⁺ |
| 207A | 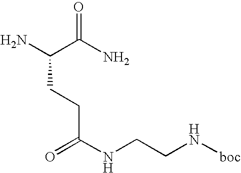 | Ex. 169A | LC-MS (Method 10): Rₜ =2.23 minMS (ESI): m/z = 289 (M + H)⁺ |
| 208A | 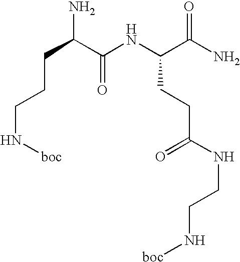 | Ex. 170A | LC-MS (Method 2): Rₜ =1.11 minMS (ESI): m/z = 503 (M + H)⁺ |
| 209A | 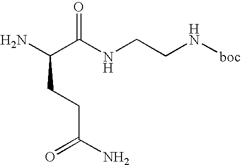 | Ex. 171A | LC-MS (Method 10): Rₜ =2.20 minMS (ESI): m/z = 289 (M + H)⁺ |
| 210A | 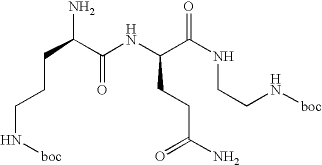 | Ex. 172A | LC-MS (Method 2): Rₜ =1.10 minMS (ESI): m/z = 503 (M + H)⁺ |
| 211A | 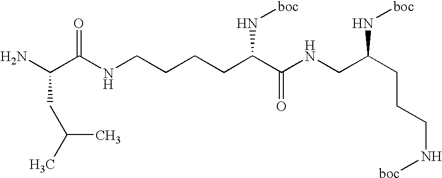 | Ex. 173A | MS (ESI): m/z = 659 (M + H)⁺ |
| 212A | 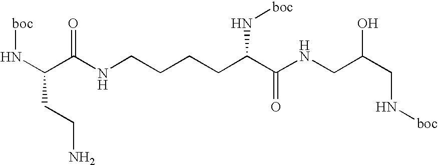 | Ex. 174A | MS (ESI): m/z = 619 (M + H)⁺ |
| 213A | 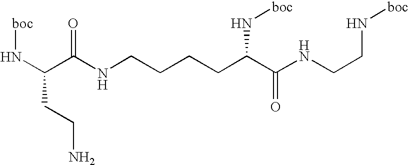 | Ex. 175A | MS (ESI): m/z = 589 (M + H)⁺ |
| 214A | 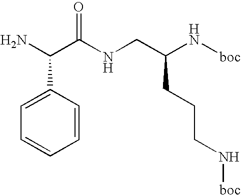 | Ex. 176A | LC-MS (Method 2): Rₜ =1.33 minMS (ESI): m/z = 451 (M + H)⁺ |
| 215A | 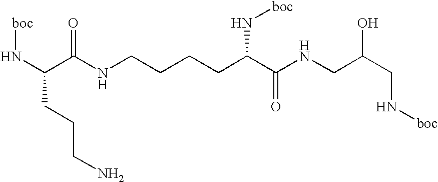 | Ex. 187A | MS (ESI): m/z = 633 (M + H)⁺ |
| 216A | 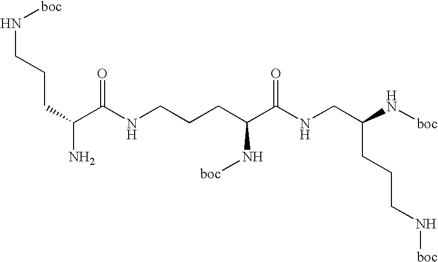 | Ex. 178A | LC-MS (Method 2): Rₜ =1.79 minMS (ESI): m/z = 746 (M + H)⁺ |
| 217A | 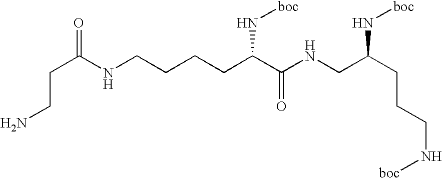 | Ex. 179A | MS (ESI): m/z = 617 (M + H)⁺ |
| 218A | 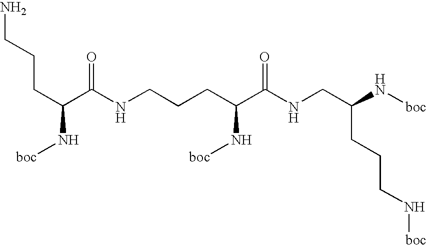 | Ex. 180A | MS (ESI): m/z = 746 (M + H)⁺ |
| 219A | 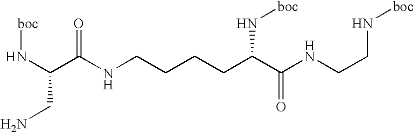 | Ex. 181A | MS (ESI): m/z = 575 (M + H)⁺ |
| 220A | 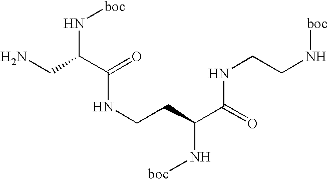 | Ex. 182A | MS (ESI): m/z = 547 (M + H)⁺ |
| 221A | 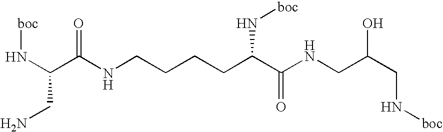 | Ex. 183A | MS (ESI): m/z = 605 (M + H)⁺ |
| 222A | 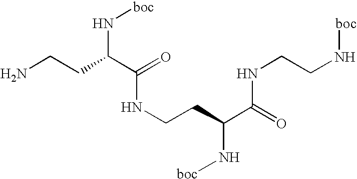 | Ex 184A | MS (ESI): m/z = 561 (M + H)⁺ |
| 223A | 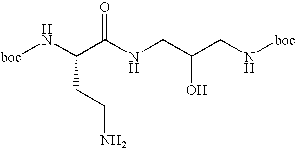 | Ex. 185A | MS (ESI): m/z = 391 (M + H)⁺ |
| 224A | 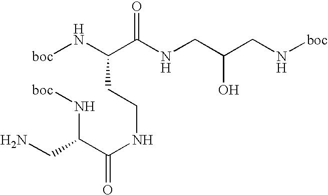 | Ex. 186A | MS (ESI): m/z = 577 (M + H)⁺ |

### Example 225A

Benzyl ((4S)-5-[(3-amino-2-hydroxypropyl)amino]-4-{[(benzyloxy)carbonyl]amino}-5-oxopentyl)carbamate hydrochloride

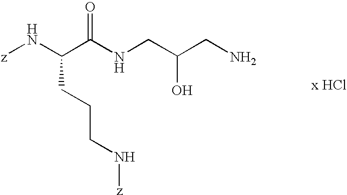

At 0° C., 6.8 ml of a 4N hydrogen chloride-dioxane solution are added to a solution of 0.263 g (0.46 mmol) of the compound from Example 187A in 1 ml of dioxane. After 2 h at RT, the reaction solution is concentrated in vacuo and coevaporated several times with dichloromethane. The remaining solid is dried to constant weight under high vacuum.

Yield: 0.205 g (88% of theory)

LC-MS (Method 2): Rₜ=1.47 min

MS (EI): m/z=473 (M−HCl+H)⁺

### Example 226A

### Benzyl [(1S)-4-{[(benzyloxy)carbonyl]amino}-1-({[3-({[(8S,11S,14S)-14-[(tert-butoxycarbonyl)amino]-11-{(2R)-3-[(tert-butoxycarbonyl)amino]-2-hydroxypropyl}-17-hydroxy-9-methyl-10,13-dioxo-9,12-diazatricyclo[14.3.1.1^{2,6}]henicosa-1(20),2(21),3,5,16,18-hexaen-8-yl]carbonyl}amino)-2-hydroxypropyl]amino}carbonyl)butyl]carbamate

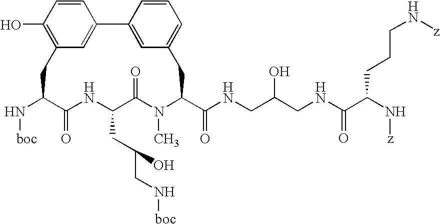

25 mg (0.037 mmol) of the compound from Example 45A are dissolved in 1.0 ml of DMF and cooled to 0° C. 21 mg (0.041 mmol) of PyBOP and 15 mg (0.11 mmol) of diisopropylamine are added. After 30 min, 24.7 mg (0.048 mmol) of the compound from Example 225A are added and the mixture is stirred for 12 h at room temperature. The reaction mixture is concentrated on a rotary evaporator in vacuo and purified by chromatography over Sephadex-LH20 (mobile phase: methanol/acetic acid 0.25%).

Yield: 12.7 mg (30% of theory)

LC-MS (Method 3): Rₜ=2.61 min

MS (ESI): m/z=1125 (M+H)⁺

### Example 227A

### tert-Butyl {(2R)-3-[(8S,11S,14S)-14-[(tert-butoxycarbonyl)amino]-17-hydroxy-8-({[2-hydroxy-3-(L-ornithylamino)propyl]amino}carbonyl)-9-methyl-10,13-dioxo-9,12-diazatricyclo[14.3.1.1^{2,6}]henicosa-1(20),2(21),3,5,16,18-hexaen-11-yl]-2-hydroxypropyl}carbamate

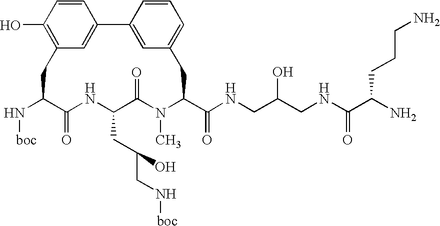

12.7 mg (0.011 mmol) of the compound from Example 226A are dissolved in 5 ml of ethanol, 5 mg of Pd/C (10%) are added and the mixture is hydrogenated for 12 h under atmospheric pressure and a hydrogen atmosphere. Suction filtration is carried out, the reaction mixture is concentrated in vacuo and the crude product is used without further purification in the next step.

Yield: 11 mg (95% of theory)

LC-MS (Method 2): Rₜ=1.26 min

MS (ESI): m/z=857 (M+H)⁺

Examples 228A and 229A listed in the following table are prepared in analogy to the procedure of Example 112A.

| | Pre- | | |
|---|---|---|---|
| Exam- | cursor | | |
| ple | Exam- | | |
| No. | ple | Structure | Analytical Data |
| 228A | 43A +198A | 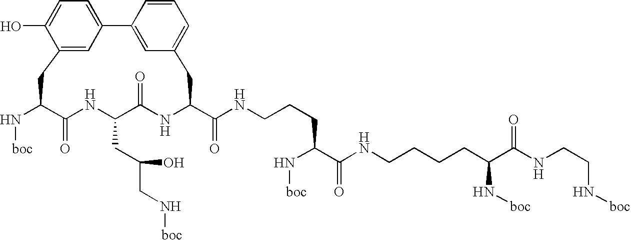 | LC-MS (Method 2): Rₜ =2.41 minMS (ESI): m/z =1241 (M + H)⁺. |
| 229A | 43A +213A | 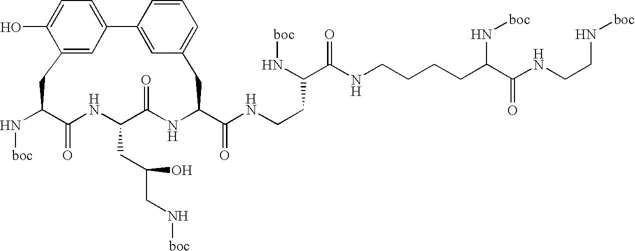 | LC-MS (Method 2): Rₜ =2.41 minMS (ESI): m/z =1227 (M + H)⁺. |

Examples 230A to 254A listed in the following table are prepared in analogy to the procedure of Example 117A.

| | Pre- | | |
|---|---|---|---|
| Exam- | cursor | | |
| ple | Exam- | | |
| No. | ple | Structure | Analytical Data |
| 230A | 44A +216A | 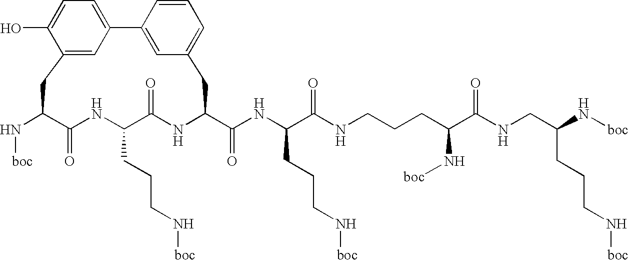 | LC-MS (Method 2): Rₜ =2.76 minMS (ESI): m/z =1368 (M + H)⁺. |
| 231A | 47A +67A | 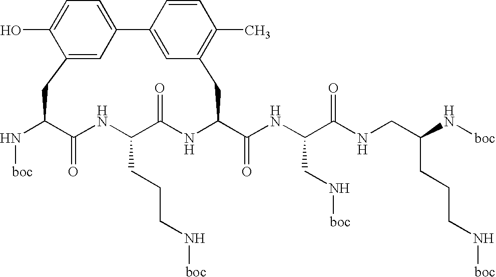 | LC-MS (Method 2): Rₜ =2.71 minMS (ESI): m/z =1140 (M + H)⁺. |
| 232A | 44A +193A | 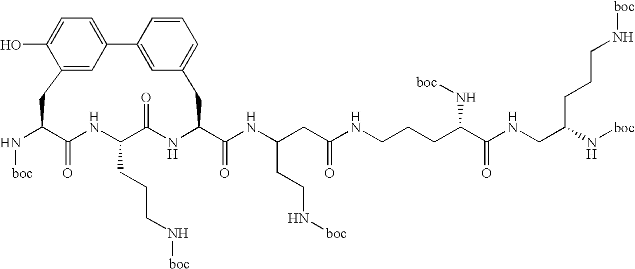 | LC-MS (Method 2): Rₜ =2.72 minMS (ESI): m/z =1368 (M + H)⁺. |
| 233A | 44A +197A | 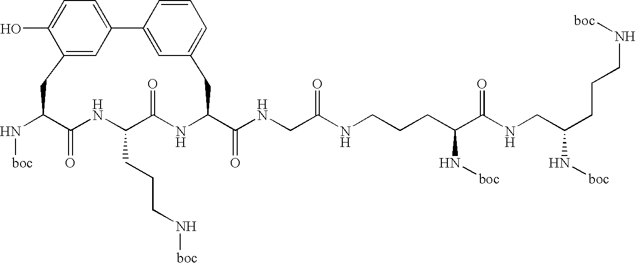 | LC-MS (Method 2): Rₜ =2.51 minMS (ESI): m/z =1211 (M + H)⁺. |
| 234A | 43A +200A | 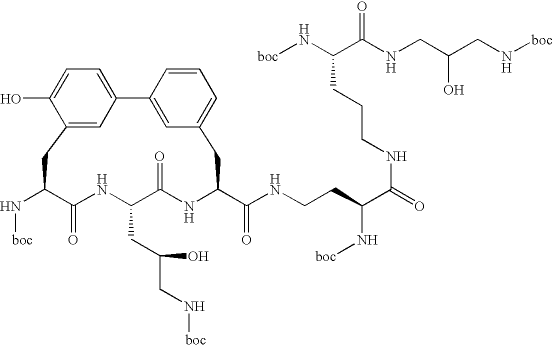 | LC-MS (Method 2): Rₜ =2.61 minMS (ESI): m/z =1243 (M + H)⁺. |
| 235A | 47A +202A | 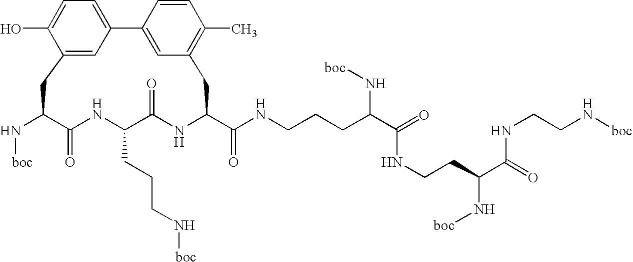 | LC-MS (Method 1): Rₜ =2.65 minMS (ESI): m/z =1211 (M + H)⁺. |
| 236A | 43A +202A | 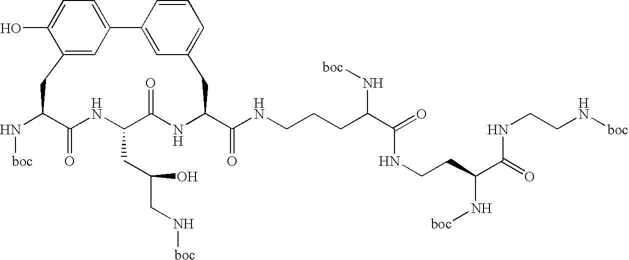 | LC-MS (Method 2): Rₜ =2.39 minMS (ESI): m/z =1213 (M + H)⁺. |
| 237A | 44A +203A | 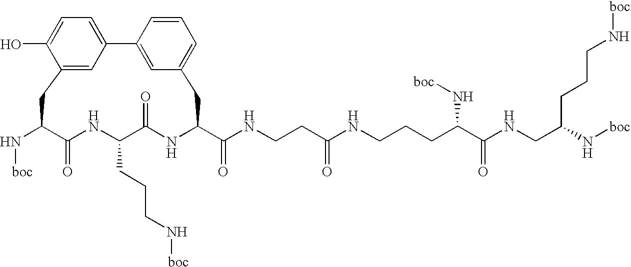 | LC-MS (Method 2): Rₜ =2.51 minMS (ESI): m/z =1225 (M + H)⁺. |
| 238A | 43A +188A | 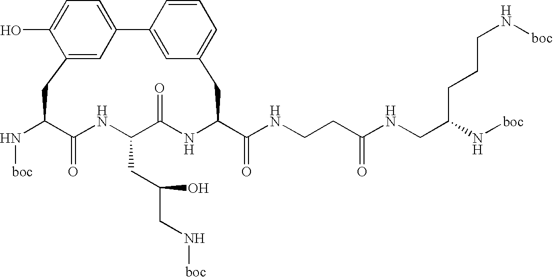 | LC-MS (Method 2): Rₜ =2.33 minMS (ESI): m/z =1027 (M + H)⁺. |
| 239A | 47A +105A | 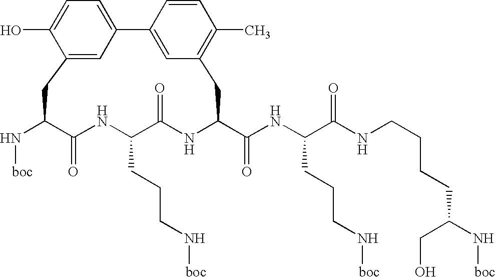 | LC-MS (Method 3): Rₜ =2.63 minMS (ESI): m/z =1083 (M + H)⁺. |
| 240A | 43A +205A | 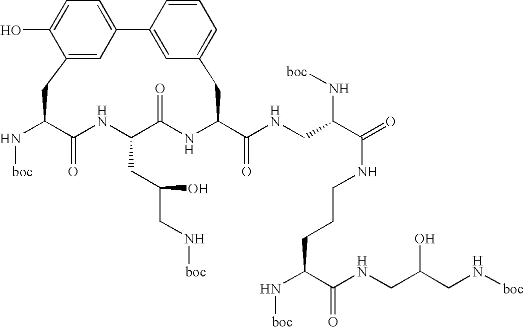 | LC-MS (Method 3): Rₜ =2.64 minMS (ESI): m/z =1229 (M + H)⁺. |
| 241A | 43A +206A | 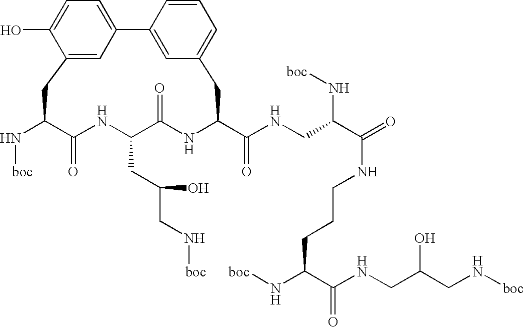 | LC-MS (Method 3): Rₜ =2.56 minMS (ESI): m/z =1257 (M + H)⁺. |
| 242A | 44A +200A | 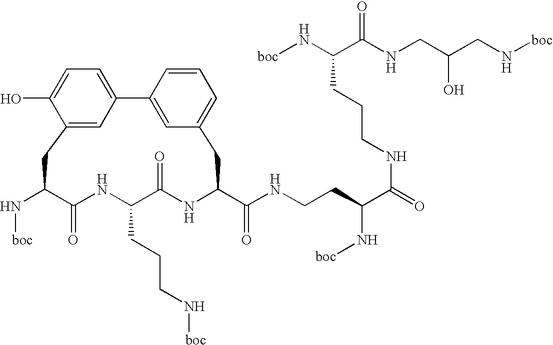 | LC-MS (Method 3): Rₜ =2.67 minMS (ESI): m/z =1227 (M + H)⁺. |
| 243A | 43A +208A | 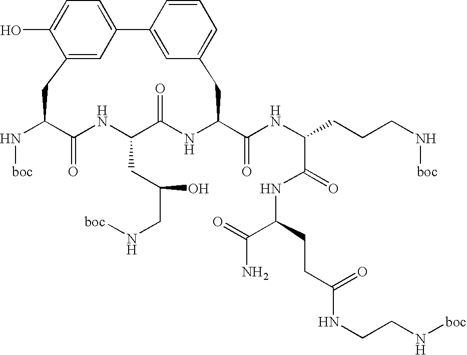 | LC-MS (Method 3): Rₜ =2.42 minMS (ESI): m/z =1141 (M + H)⁺. |
| 244A | 43A +210A | 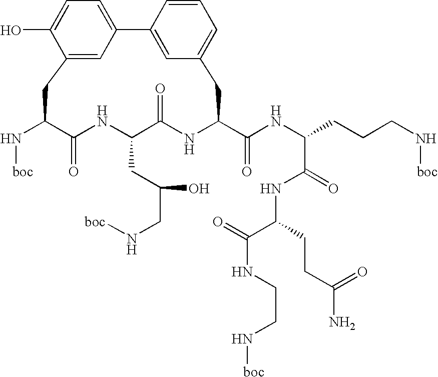 | LC-MS (Method 3): Rₜ =2.42 minMS (ESI): m/z =1141 (M + H)⁺. |
| 245A | 47A +208A | 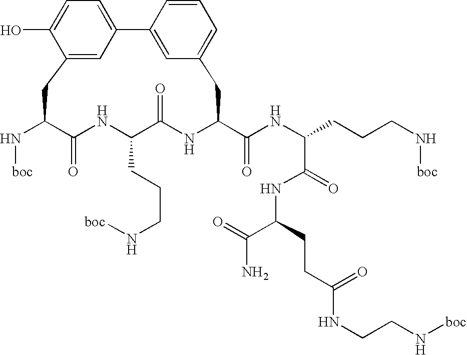 | LC-MS (Method 3): Rₜ =2.51 minMS (ESI): m/z =1139 (M + H)⁺. |
| 246A | 47A +210A | 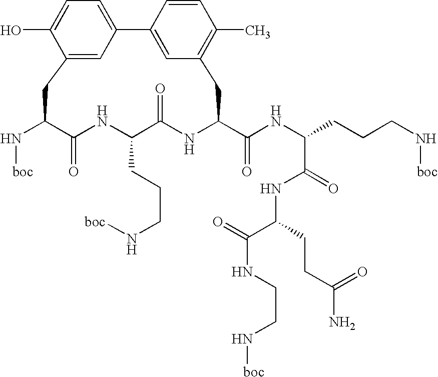 | LC-MS (Method 3): Rₜ =2.51 minMS (ESI): m/z =1139 (M + H)⁺. |
| 247A | 44A +210A | 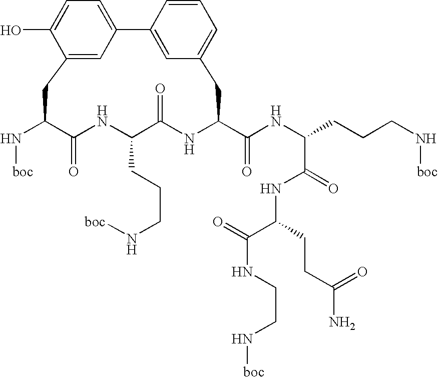 | LC-MS (Method 3): Rₜ =2.46 minMS (ESI): m/z =1125 (M + H)⁺. |
| 248A | 43A +222A | 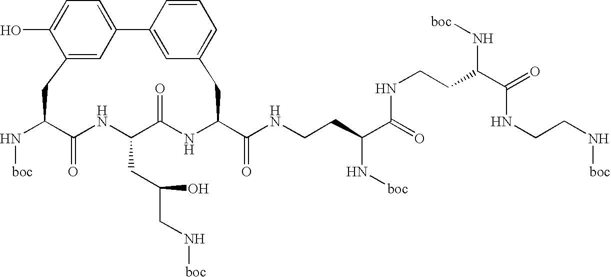 | LC-MS (Method 3): Rₜ =2.63 minMS (ESI): m/z =1199 (M + H)⁺. |
| 249A | 47A +206A | 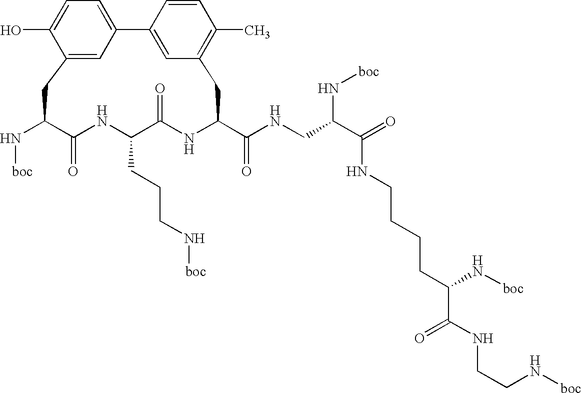 | LC-MS (Method 1): Rₜ =2.72 minMS (ESI): m/z =1211 (M + H)⁺. |
| 250A | 44A +206A | 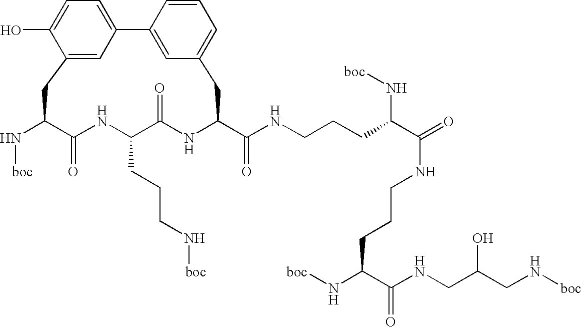 | LC-MS (Method 3): Rₜ =2.65 minMS (ESI): m/z =1241 (M + H)⁺. |
| 251A | 47A +221A | 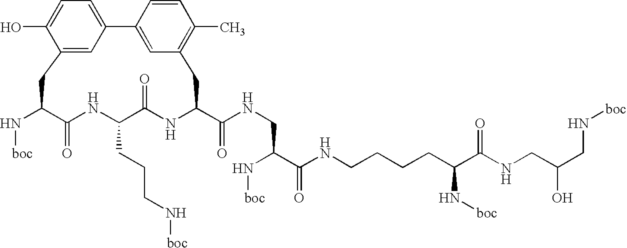 | LC-MS (Method 1): Rₜ =2.61 minMS (ESI): m/z =1241 (M + H)⁺. |
| 252A | 44A +222A | 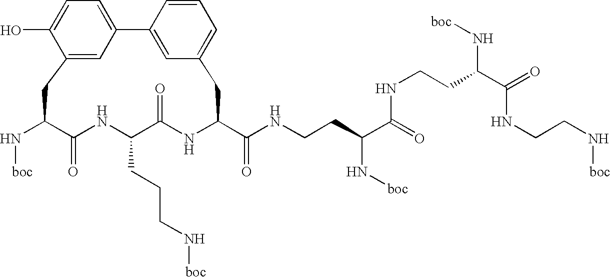 | LC-MS (Method 3): Rₜ =2.71 minMS (ESI): m/z =1183 (M + H)⁺. |
| 253A | 47A +224A | 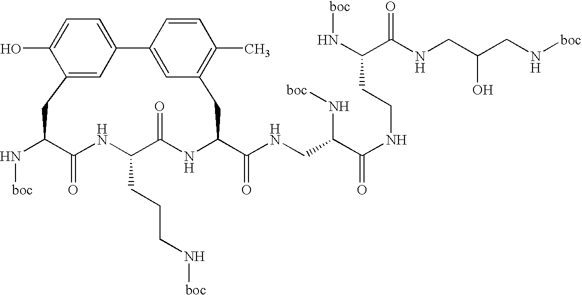 | LC-MS (Method 1): Rₜ =2.60 minMS (ESI): m/z =1199 (M + H)⁺. |
| 254A | 44A +208A | 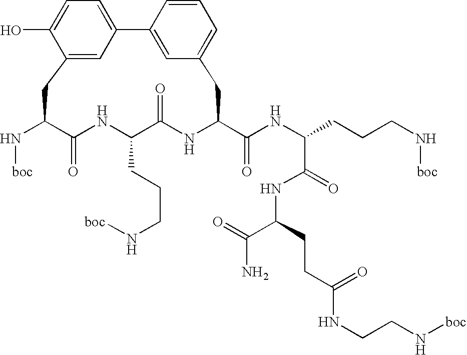 | LC-MS (Method 3): Rₜ =2.45 minMS (ESI): m/z =1125 (M + H)⁺. |

Examples 255A to 281A listed in the following table are prepared in analogy to the procedure of Example 113A.

| | Pre- | | |
|---|---|---|---|
| Exam- | cursor | | |
| ple | Exam- | | |
| No. | ple | Structure | Analytical Data |
| 255A | 47A +57A | 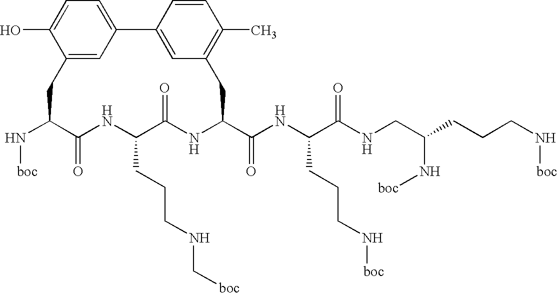 | LC-MS (Method 2): Rₜ = 2.73 minMS (ESI): m/z = 1168 (M + H)⁺. |
| 256A | 45A +188A | 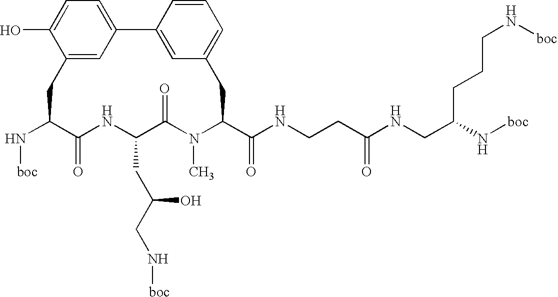 | LC-MS (Method 2): Rₜ = 2.42 minMS (ESI): m/z = 1041 (M + H)⁺. |
| 257A | 47A +189A | 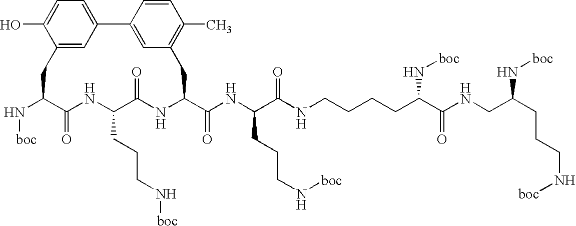 | LC-MS (Method 3): Rₜ = 3.02 minMS (ESI): m/z = 1396 (M + H)⁺. |
| 258A | 43A +194A | 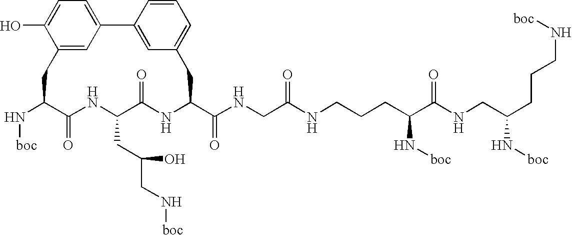 | LC-MS (Method 3): Rₜ = 2.65 min.MS (ESI): m/z = 1241 (M + H)⁺. |
| 259A | 43A +189A | 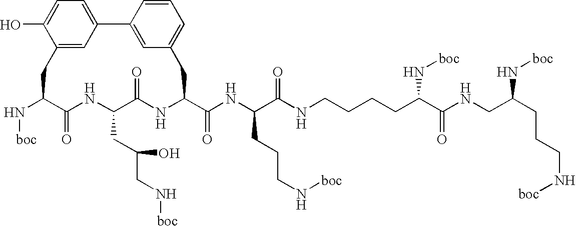 | LC-MS (Method 3): Rₜ = 2.90 minMS (ESI): m/z = 1398 (M + H)⁺. |
| 260A | 44A +189A | 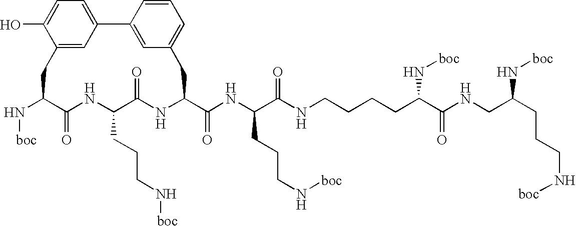 | LC-MS (Method 3): Rₜ = 2.96 minMS (ESI): m/z = 1382 (M + H)⁺. |
| 261A | 44A +192A | 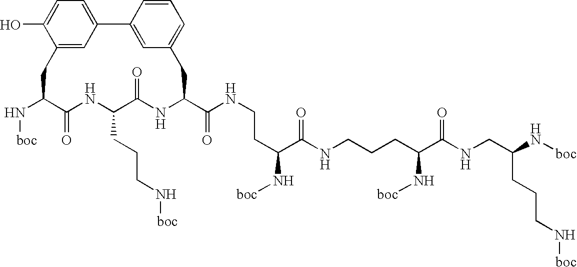 | LC-MS (Method 2): Rₜ = 2.67 minMS (ESI): m/z = 1354 (M + H)⁺. |
| 262A | 43A +217A | 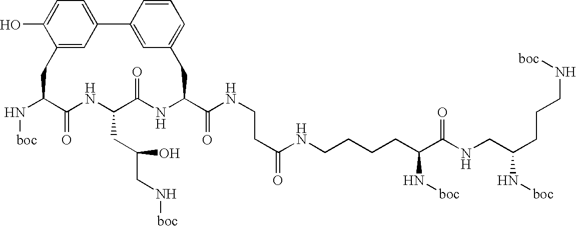 | LC-MS (Method 3): Rₜ = 2.63 minMS (ESI): m/z = 1255 (M + H)⁺. |
| 263A | 47A +217A | 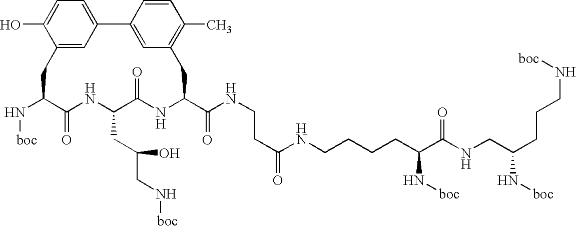 | LC-MS (Method 2): Rₜ = 2.57 minMS (ESI): m/z = 1253 (M + H)⁺. |
| 264A | 44A +218A | 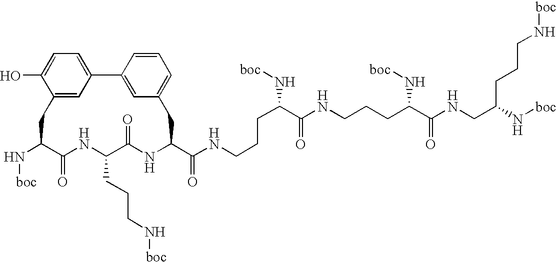 | LC-MS (Method 3): Rₜ = 2.95 minMS (ESI): m/z = 1368 (M + H)⁺. |
| 265A | 43A +218A | 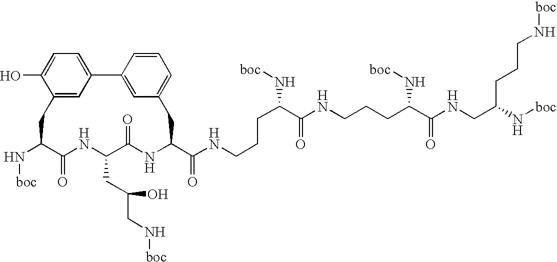 | LC-MS (Method 3): Rₜ = 2.90 minMS (ESI): m/z = 1384 (M + H)⁺. |
| 266A | 44A +194A | 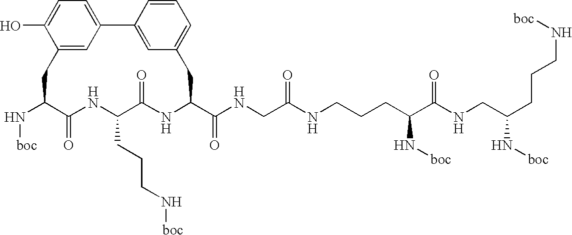 | LC-MS (Method 2): Rₜ = 2.52 minMS (ESI): m/z = 1225 (M + H)⁺. |
| 267A | 45A +195A | 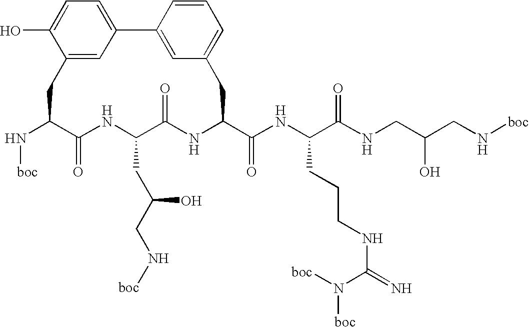 | LC-MS (Method 3): Rₜ = 2.96 minMS (ESI): m/z = 1199 (M + H)⁺. |
| 268A | 45A +196A | 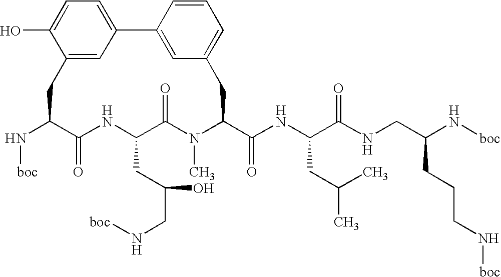 | LC-MS (Method 3): Rₜ = 2.87 minMS (ESI): m/z = 1083 (M + H)⁺. |
| 269A | 43A +191A | 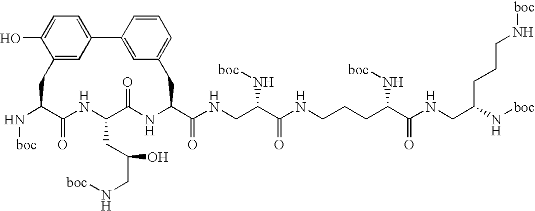 | LC-MS (Method 2): Rₜ = 2.66 minMS (ESI): m/z = 1356 (M + H)⁺. |
| 270A | 43A +204A | 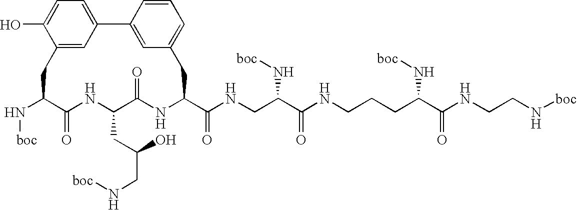 | LC-MS (Method 3): Rₜ = 2.18 minMS (ESI): m/z = 1199 (M + H)⁺. |
| 271A | 43A +192A | 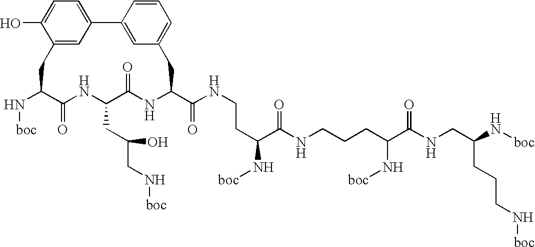 | LC-MS (Method 3): Rₜ = 2.88 minMS (ESI): m/z = 1370 (M + H)⁺. |
| 272A | 43A +211A | 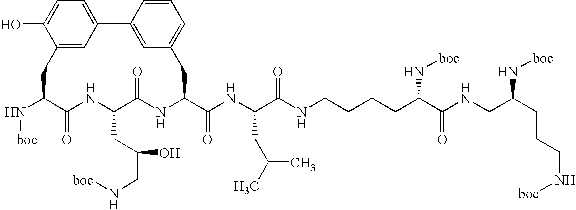 | LC-MS (Method 3): Rₜ = 2.87 minMS (ESI): m/z = 1297 (M + H)⁺. |
| 273A | 45A +71A | 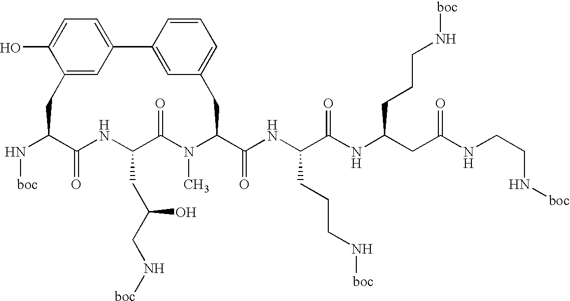 | LC-MS (Method 2): Rₜ = 2.56 minMS (ESI): m/z = 1255 (M + H)⁺. |
| 274A | 43A +212A | 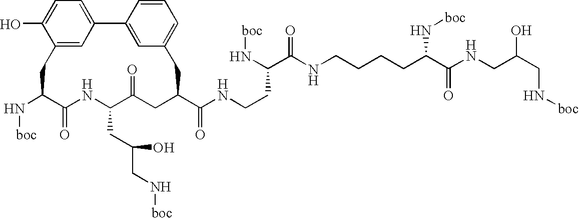 | LC-MS (Method 2): Rₜ = 2.35 minMS (ESI): m/z = 1257 (M + H)⁺. |
| 275A | 44A +191A | 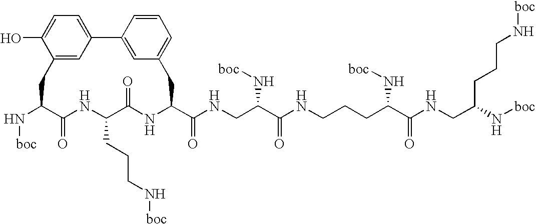 | LC-MS (Method 2): Rₜ = 2.71 minMS (ESI): m/z = 1340 (M + H)⁺. |
| 276A | 45A +214A | 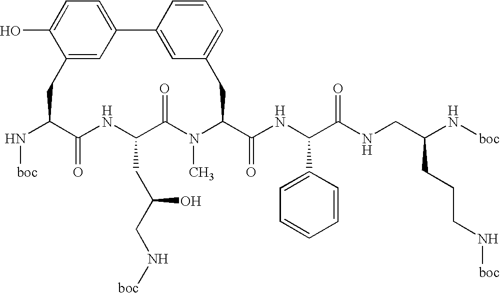 | LC-MS (Method 3): Rₜ = 2.81 minMS (ESI): m/z = 1103 (M + H)⁺. |
| 277A | 44A +217A | 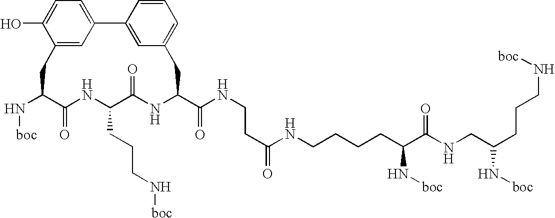 | LC-MS (Method 2): Rₜ = 2.52 minMS (ESI): m/z = 1239 (M + H)⁺. |
| 278A | 44A +198A | 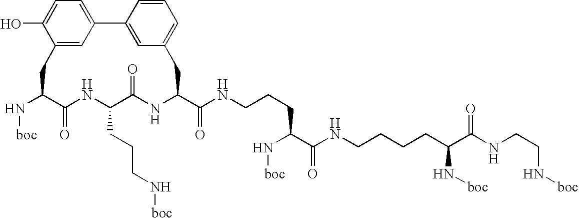 | LC-MS (Method 1): Rₜ = 2.61 minMS (ESI): m/z = 1225 (M + H)⁺. |
| 279A | 47A +204A | 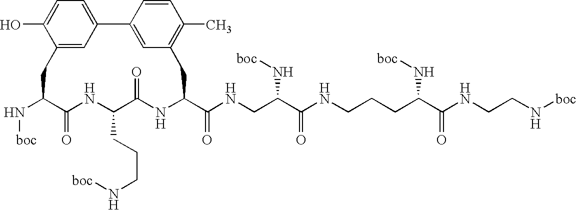 | LC-MS (Method 2): Rₜ = 2.52 minMS (ESI): m/z = 1239 (M + H)⁺. |
| 280A | 43A +220A | 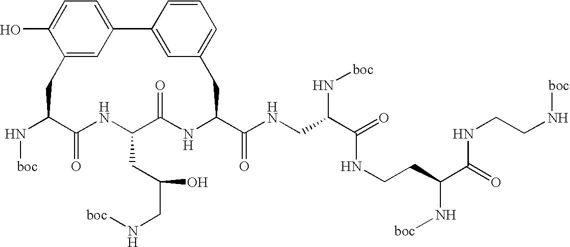 | LC-MS (Method 1): Rₜ = 2.57 minMS (ESI): m/z = 1185 (M + H)⁺. |
| 281A | 43A +215A | 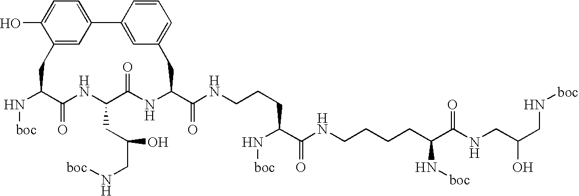 | LC-MS (Method 3): Rₜ = 2.52 minMS (ESI): m/z = 1271 (M + H)⁺. |

### Exemplary Embodiments

### Example 1

### (8S,11S,14S)-14-Amino-N-((1S)-4-amino-1-{[(2-aminoethyl)amino]carbonyl)}butyl)-11-[(2R)-3-amino-2-hydroxypropyl]-17-hydroxy-10,13-dioxo-9,12-diazatricyclo[14.3.1.1^{2,6}]-henicosa-1 (20),2(21),3,5,16,18-hexaene-8-carboxamide tetrahydrochloride

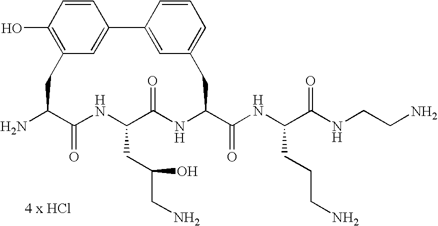

At 0° C., 0.084 ml of a 4N hydrogen chloride-dioxane solution are added to a solution of 5.7 mg (0.006 mmol) of the compound from Example 120A in 1 ml of dioxane. After 2 h at RT, the reaction solution is concentrated in vacuo and coevaporated several times with dichloromethane. The remaining solid is dried to constant weight under high vacuum.

Yield: 3.3 mg (77% of theory)

MS (ESI): m/z=612 (M-4HCl+H)⁺.

### Example 2

### (8S,11S,14S)-14-Amino-11-[(2R)-3-amino-2-hydroxypropyl]-N-(2-{[(2S)-2,5-diaminopentyl]-amino}-2-oxoethyl)-17-hydroxy-10,13-dioxo-9,12-diazatricyclo[14.3.1.1^{2,6}]henicosa-1(20),2(21),3,5,16,18-hexaene-8-carboxamide tetrahydrochloride

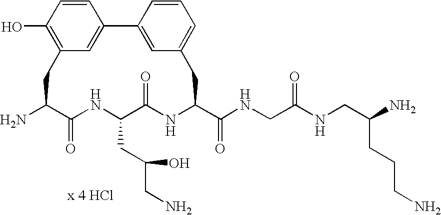

At 0° C., 0.062 ml of a 4N hydrogen chloride-dioxane solution are added to a solution of 4.2 mg (0.004 mmol) of the compound from Example 121A in 1 ml of dioxane. After 3 h at RT, the reaction solution is concentrated in vacuo and coevaporated several times with dichloromethane. The remaining solid is dried to constant weight under high vacuum.

Yield: 2 mg (64% of theory)

MS (ESI): m/z=613 (M-4HCl+H)⁺.

### Example 3

### (8S,11S,14S)-14-Amino-N-[(s)-4-amino-1-({[(25)-2,5-diaminopentyl]amino}carbonyl)butyl]-11-(3-aminopropyl)-17-hydroxy-10,13-dioxo-9,12-diazatricyclo[14.3.1.1^{2,6}]henicosa-1(20),2(21),3,5,16,18-hexaene-8-carboxamide pentahydrochloride

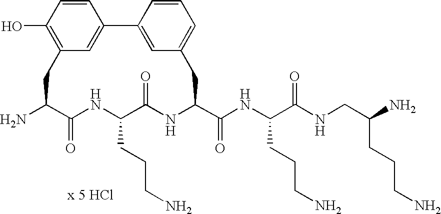

At 0° C., 0.4 ml of a 4N hydrogen chloride-dioxane solution are added to a solution of 22.8 mg (0.02 mmol) of the compound from Example 113A in 1 ml of dioxane. After 3 h at RT, the reaction solution is concentrated in vacuo and coevaporated several times with dichloromethane. The remaining solid is dried to constant weight under high vacuum.

Yield: 15.3 mg (93% of theory)

MS (ESI): m/z=654 (M-5HCl+H)⁺.

¹H-NMR (400 MHz, D₂O): δ=1.55-1.95 (m, 12H), 2.8-3.2 (m, 9H), 3.3-3.7 (m, 4H), 4.29 (m_{c}, 1H), 4.47 (m_{c}, 1H), 4.7-4.9 (m, 2H, under D₂O), 6.94 (d, 1H), 6.99 (s, 1H), 7.16 (d, 1H), 7.31 (s, 1H), 7.35 (t, 1H), 7.4-7.5 (m, 2H).

### Example 4

### (8S,11S,14S)-14-Amino-N-[(15)-4-amino-1-({[(25)-2,5-diaminopentyl]amino}carbonyl)butyl]-11-(3-aminopropyl)-17-hydroxy-10,13-dioxo-9,12-diazatricyclo[14.3.1.1^{2,6}]henicosa-1 (20),2(21),3,5,16,18-hexaene-8-carboxamide penta(hydrotrifluoroacetate)

Example 3 as tetrahydrochloride salt is converted by preparative HPLC (Reprosil ODS-A, mobile phase acetonitrile/0.2% aqueous trifluoroacetic acid 5:95→95:5) into the tetra(hydrotrifluoroacetate).

LC-MS (Method 10): Rₜ=2.21 min

MS (ESI): m/z=654 (M-5TFA+H)⁺.

### Example 5

### (8S,11S,14S)-14-Amino-N-{(4S)-4-amino-5-[(2-aminoethyl)amino]-5-oxopentyl}-11-[(2R)-3-amino-2-hydroxypropyl]-17-hydroxy-10,13-dioxo-9,12-diazatricyclo[14.3.1.1^{2,6}]-henicosa-1(20),2(21),3,5,16,18-hexaene-8-carboxamide tetrahydrochloride

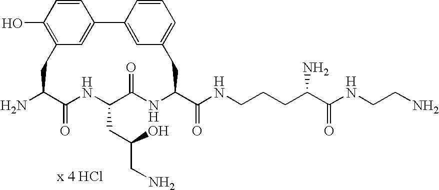

At 0° C., 0.27 ml of a 4N hydrogen chloride-dioxane solution are added to a solution of 4.6 mg (0.005 mmol) of the compound from Example 117A in 1 ml of dioxane. After 3 h at RT, the reaction solution is concentrated in vacuo and coevaporated several times with dichloromethane. The remaining solid is dried to constant weight under high vacuum.

Yield: 3.4 mg (99% of theory)

MS (ESI): m/z=613 (M-4HCl+H)⁺.

¹H-NMR (400 MHz, D₂O): δ=1.47-1.67 (m, 2H), 1.75-2.09 (m, 4H), 2.89 (m_{c}, 1H), 2.95-3.25 (m, 7H), 3.3 (m_{c}, 1H), 3.4 (m_{c}, 1H), 3.5-3.7 (m, 2H), 3.86 (m_{c}, 1H), 3.98 (m_{c}, 1H), 4.44 (m_{c}, 1H), 4.7-4.9 (m, 2H, under D₂O), 6.94 (d, 1H), 6.99 (s, 1H), 7.16 (d, 1H), 7.31 (s, 1H), 7.35 (t, 1H), 7.4-7.5 (m, 2H).

### Example 6

### (8S,11S,14S)-14-Amino-N-[(1S)-4-amino-1-({[(5S)-5-amino-6-hydroxyhexyl]amino}carbonyl)butyl]-11-(3-aminopropyl)-17-hydroxy-10,13-dioxo-9,12-diazatricyclo[14.3.1.1^{2,6}]henicosa-1 (20),2(21),3,5,16,18-hexaene-8-carboxamide tetrahydrochloride

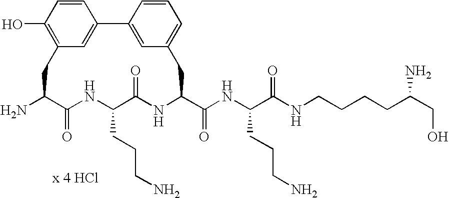

At 0° C., 0.87 ml of a 4N hydrogen chloride-dioxane solution are added to a solution of 62 mg (0.058 mmol) of the compound from Example 128A in 1 ml of dioxane. After 3 h at RT, the reaction solution is concentrated in vacuo and coevaporated several times with dichloromethane. The remaining solid is dried to constant weight under high vacuum.

Yield: 46 mg (97% of theory)

LC-MS (Method 10): Rₜ=1.84 min

MS (ESI): m/z=669 (M-4HCl+H)⁺.

¹H-NMR (400 MHz, D₂O): δ=1.25-1.95 (m, 14H), 2.9-3.3 (m, 10H), 3.5-3.8 (m, 3H), 4.19 (m_{c}, 1H), 4.46 (m_{c}, 1H), 4.7-4.9 (m, 2H, under D₂O), 6.94 (d, 1H), 6.99 (s, 1H), 7.16 (d, 1H), 7.31 (s, 1H), 7.35 (t, 1H), 7.4-7.5 (m, 2H).

### Example 7

### (8S,15S,14S)-14-Amino-N-((1S)-1-(aminomethyl)-2-{[(2S)-2,5-diaminopentyl]amino}-2-oxoethyl)-11-(3-aminopropyl)-17-hydroxy-10,13-dioxo-9,12-diazatricyclo[14.3.1.1^{2,6}]henicosa-1 (20),2(21),3,5,16,18-hexaene-8-carboxamide pentahydrochloride

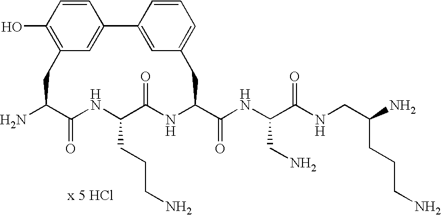

At 0° C., 0.94 ml of a 4N hydrogen chloride-dioxane solution are added to a solution of 70 mg (0.062 mmol) of the compound from Example 129A in 1 ml of dioxane. After 3 h at RT, the reaction solution is concentrated in vacuo and coevaporated several times with dichloromethane. The remaining solid is dried to constant weight under high vacuum.

Yield: 50 mg (99% of theory)

MS (ESI): m/z=626 (M-5HCl+H)⁺.

¹H-NMR (400 MHz, D₂O): δ=1.55-1.95 (m, 8H), 2.9-3.2 (m, 6H), 3.26 (m_{c}, 1H), 3.3-3.7 (m, 7H), 4.47 (m_{c}, 1H), 4.7-4.9 (m, 2H, under D₂O), 6.94 (d, 1H), 6.99 (s, 1H), 7.16 (d, 1H), 7.31 (s, 1H), 7.35 (t, 1H), 7.4-7.5 (m, 2H).

### Example 8

### (8S,11S,14S)-14-Amino-N-((1S)-4-amino-1-{[(2-aminoethyl)amino]carbonyl}butyl)-11-(3-aminopropyl)-17-hydroxy-10,13-dioxo-9,12-diazatricyclo[14.3.1.1^{2,6}]henicosa-1 (20),2(21),3,5,16,18-hexaene-8-carboxamide tetrahydrochloride

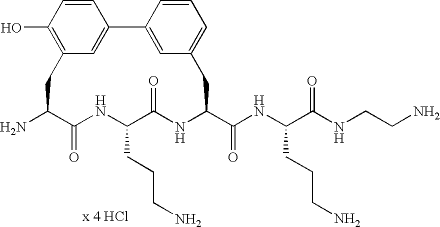

At 0° C., 0.181 ml of a 4N hydrogen chloride-dioxane solution are added to a solution of 12 mg (0.012 mmol) of the compound from Example 130A in 1 ml of dioxane. After 3 h at RT, the reaction solution is concentrated in vacuo and coevaporated several times with dichloromethane. The remaining solid is dried to constant weight under high vacuum.

Yield: 8.8 mg (99% of theory)

MS (ESI): m/z=597 (M-4HCl+H)⁺.

¹H-NMR (400 MHz, D₂O): δ=1.55-1.95 (m, 8H), 2.9-3.2 (m, 8H), 3.4-3.7 (m, 4H), 4.25 (m_{c}, 1H), 4.46 (m_{c}, 1H), 4.7-4.9 (m, 2H, under D₂O), 6.94 (d, 1H), 6.99 (s, 1H), 7.17 (d, 1H), 7.32 (s, 1H), 7.35 (t, 1H), 7.4-7.5 (m, 2H).

### Example 9

### (8S,11S,14S)-14-Amino-N-((1S)-4-amino-1-{[((1S)-4-amino-1-{2-[(2-aminoethyl)amino]-2-oxoethyl}butyl)amino]carbonyl}butyl)-11-(3-aminopropyl)-17-hydroxy-10,13-dioxo-9,12-diazatricyclo[14.3.1.1^{2,6}]henicosa-1(20),2(21),3,5,16,18-hexaene-8-carboxamide pentahydrochloride

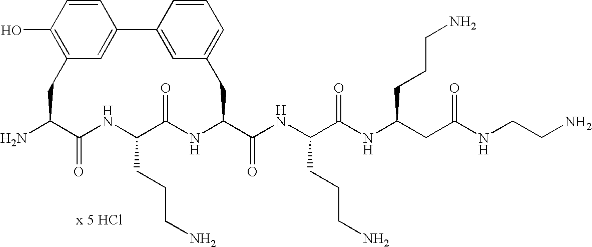

At 0° C., 0.29 ml of a 4N hydrogen chloride-dioxane solution are added to a solution of 24 mg (0.02 mmol) of the compound from Example 133A in 1 ml of dioxane. After 3 h at RT, the reaction solution is concentrated in vacuo and coevaporated several times with dichloromethane. The remaining solid is dried to constant weight under high vacuum.

Yield: 17.5 mg (99% of theory)

MS (ESI): m/z=725 (M-5HCl+H)⁺.

¹H-NMR (400 MHz, D₂O): δ=1.45-2.0 (m, 12H), 2.36 (m_{c}, 1H), 2.9-3.2 (m, 11H), 3.4-3.7 (m, 4H), 4.1-4.25 (m, 2H), 4.47 (m_{c}, 1H), 4.7-4.9 (m, 2H, under D₂O), 6.94 (d, 1H), 6.98 (s, 1H), 7.17 (d, 1H), 7.32 (s, 1H), 7.35 (t, 1H), 7.4-7.5 (m, 2H).

### Example 10

### (8S,11S,14S)-14-Amino-N-((1S)-4-amino-1-{[((1S)-4-amino-1-{2-[(2-aminoethyl)amino]-2-oxoethyl}butyl)amino]carbonyl}butyl)-11-[(2R)-3-amino-2-hydroxypropyl]-17-hydroxy-10,13-dioxo-9,12-diazatricyclo[14.3.1.1²⁶]henicosa-1(20),2(21),3,5,16,18-hexaene-8-carboxamide pentahydrochloride

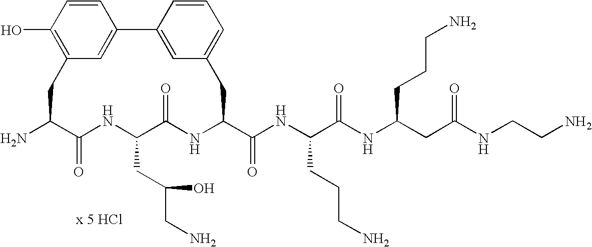

At 0° C., 0.16 ml of a 4N hydrogen chloride-dioxane solution are added to a solution of 13 mg (0.01 mmol) of the compound from Example 134A in 1 ml of dioxane. After 3 h at RT, the reaction solution is concentrated in vacuo and coevaporated several times with dichloromethane. The remaining solid is dried to constant weight under high vacuum.

Yield: 9.5 mg (99% of theory)

MS (ESI): m/z=741 (M-5HCl+H)⁺.

¹H-NMR (400 MHz, D₂O): δ=1.4-2.05 (m, 10H), 2.37 (m_{c}, 1H), 2.53 (m_{c}, 1H), 2.8-3.2 (m, 10H), 3.3-3.7 (m, 3H), 3.86 (m_{c}, 1H), 4.1-4.21 (m, 2H), 4.44 (m_{c}, 1H), 4.7-4.9 (m, 2H, under D₂O), 6.95 (d, 1H), 7.0 (s, 1H), 7.18 (d, 1H), 7.3-7.4 (m, 2H), 7.4-7.5 (m, 2H).

### Example 11

### (8S,11S,145)-14-Amino-N-{(1S)-4-amino-1-[({(4S)-4-amino-6-[(2-aminoethyl)amino]-6-oxohexyl}amino)carbonyl]butyl}-11-[(2R)-3-amino-2-hydroxypropyl]-17-hydroxy-10,13-dioxo-9,12-diazatricyclo[14.3.1.1^{2,6}]henicosa-1(20),2(21),3,5,16,18-hexaene-8-carboxamide pentahydrochloride

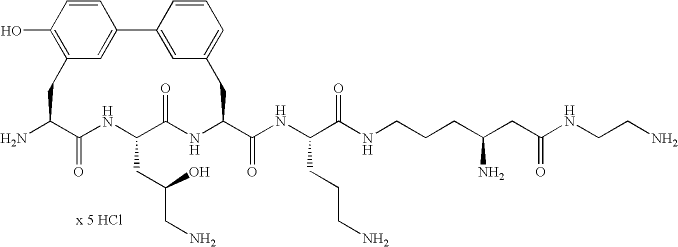

At 0° C., 0.29 ml of a 4N hydrogen chloride-dioxane solution are added to a solution of 24 mg (0.02 mmol) of the compound from Example 135A in 1 ml of dioxane. After 3 h at RT, the reaction solution is concentrated in vacuo and coevaporated several times with dichloromethane. The remaining solid is dried to constant weight under high vacuum.

Yield: 17.5 mg (99% of theory)

MS (ESI): m/z=741 (M-5HCl+H)⁺.

¹H-NMR (400 MHz, D₂O): δ=1.45-2.05 (m, 10H), 2.55 (m_{c}, 1H), 2.68 (m_{c}, 1H), 2.8-3.2 (m, 10H), 3.3-3.7 (m, 4H), 3.86 (m_{c}, 1H), 4.21 (m_{c}, 2H), 4.44 (m_{c}, 1H), 4.7-4.9 (m, 2H, under D₂O), 6.94 (d, 1H), 6.99 (s, 1H), 7.17 (d, 1H), 7.33 (s, 1H), 7.35 (t, 1H), 7.4-7.5 (m, 2H).

### Example 12

### (8S,11S,14S)-14-Amino-N-{(1S)-4-amino-1-[({(4S)-4-amino-6-[(2-aminoethyl)amino]-6-oxohexyl}amino)carbonyl]butyl}-11-(3-aminopropyl)-17-hydroxy-10,13-dioxo-9,12-diazatricyclo[14.3.1.1^{2,6}]henicosa-1(20),2(21),3,5,16,18-hexaene-8-carboxamide pentahydrochloride

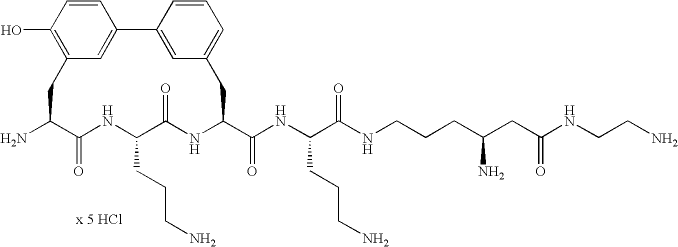

At 0° C., 0.26 ml of a 4N hydrogen chloride-dioxane solution are added to a solution of 21 mg (0.017 mmol) of the compound from Example 136A in 1 ml of dioxane. After 3 h at RT, the reaction solution is concentrated in vacuo and coevaporated several times with dichloromethane. The remaining solid is dried to constant weight under high vacuum.

Yield: 15 mg (99% of theory)

MS (ESI): m/z=716 (M-5HCl+H)⁺.

¹H-NMR (400 MHz, D₂O): δ=1.45-1.95 (m, 12H), 2.55 (m_{c}, 1H), 2.68 (m_{c}, 1H), 2.9-3.2 (m, 10H), 3.42 (m_{c}, 2H), 3.5-3.7 (m, 3H), 4.2 (m_{c}, 1H), 4.46 (m_{c}, 1H), 4.7-4.9 (m, 2H, under D₂O), 6.94 (d, 1H), 6.98 (s, 1H), 7.17 (d, 1H), 7.32 (s, 1H), 7.35 (t, 1H), 7.4-7.5 (m, 2H).

### Example 13

### (8S,11S,14S)-14-Amino-N-[(1S)-4-amino-1-({[(2S)-2,5-diaminopentyl]amino}carbonyl)butyl]-11-[(2R)-3-amino-2-hydroxypropyl]-17-hydroxy-10,13-dioxo-9,12-diazatricyclo[14.3.1.1^{2,6}]henicosa-1 (20),2(21),3,5,16,18-hexaene-8-carboxamide pentahydrochloride

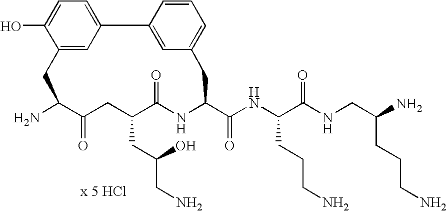

At 0° C., 0.256 ml of a 4N hydrogen chloride-dioxane solution are added to a solution of 20 mg (0.017 mmol) of the compound from Example 137A in 1 ml of dioxane. After 3 h at RT, the reaction solution is concentrated in vacuo and coevaporated several times with dichloromethane. The remaining solid is dried to constant weight under high vacuum.

Yield: 13.5 mg (93% of theory)

MS (ESI): m/z=670 (M-5HCl+H)⁺.

¹H-NMR (400 MHz, D₂O): δ=1.5-2.05 (m, 10H), 2.8-3.2 (m, 8H), 3.3-3.7 (m, 5H), 3.86 (m_{c}, 1H), 4.30 (m_{c}, 1H), 4.44 (m_{c}, 1H), 4.7-4.9 (m, 2H, under D₂O), 6.94 (d, 1H), 6.99 (s, 1H), 7.17 (d, 1H), 7.33 (s, 1H), 7.35 (t, 1H), 7.4-7.5 (m, 2H).

### Example 14

### (8S,11S,14S)-14-Amino-N-((1S)-4-amino-1-{[((4S)-4-amino-6-{[(25)-2,5-diaminopentyl]amino}-6-oxohexyl)amino]carbonyl}butyl)-11-[(2R)-3-amino-2-hydroxypropyl]-17-hydroxy-10,13-dioxo-9,12-diazatricyclo[14.3.1.1^{2,6}]henicosa-1(20),2(21),3,5,16,18-hexaene-8-carboxamide hexahydrochloride

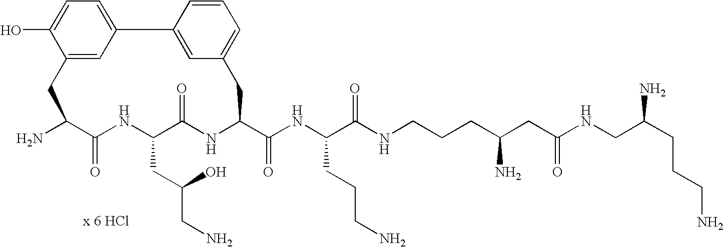

At 0° C., 0.31 ml of a 4N hydrogen chloride-dioxane solution are added to a solution of 29 mg (0.021 mmol) of the compound from Example 138A in 1 ml of dioxane. After 3 h at RT, the reaction solution is concentrated in vacuo and coevaporated several times with dichloromethane. The remaining solid is dried to constant weight under high vacuum.

Yield: 16.5 mg (78% of theory)

MS (ESI): m/z=798 (M-6HCl+H)⁺.

¹H-NMR (400 MHz, D₂O): δ=1.45-2.05 (m, 14H), 2.50 (m_{c}, 1H), 2.72 (m_{c}, 1H), 2.8-3.7 (m, 15H), 3.89 (m_{c}, 1H), 4.23 (m_{c}, 1H), 4.46 (m_{c}, 1H), 4.7-4.9 (m, 2H, under D₂O), 6.94 (d, 1H), 6.99 (s, 1H), 7.17 (d, 1H), 7.33 (s, 1H), 7.35 (t, 1H), 7.4-7.5 (m, 2H).

### Example 15

### (8S,11S,14S)-14-Amino-N-((1S)-4-amino-1-{[((4S)-4-amino-6-{[(2S)-2,5-diaminopentyl]-amino}-6-oxohexyl)amino]carbonyl}butyl)-11-(3-aminopropyl)-17-hydroxy-10,13-dioxo-9,12-diazatricyclo[14.3.1.1^{2,6}]henicosa-1 (20),2(21),3,5,16,18-hexaene-8-carboxamide hexahydrochloride

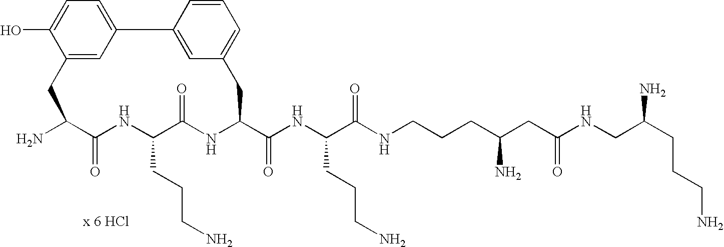

At 0° C., 0.31 ml of a 4N hydrogen chloride-dioxane solution are added to a solution of 29 mg (0.021 mmol) of the compound from Example 139A in 1 ml of dioxane. After 3 h at RT, the reaction solution is concentrated in vacuo and coevaporated several times with dichloromethane. The remaining solid is dried to constant weight under high vacuum.

Yield: 16.5 mg (78% of theory)

MS (ESI): m/z=782 (M-6HCl+H)⁺.

¹H-NMR (400 MHz, D₂O): δ=1.45-1.95 (m, 16H), 2.60 (m_{c}, 1H), 2.83 (m_{c}, 1H), 2.9-3.3 (m, 10H), 3.3-3.75 (m, 6H), 4.24 (m_{c}, 1H), 4.49 (m_{c}, 1H), 4.7-4.9 (m, 2H, under D₂O), 6.94 (d, 1H), 6.99 (s, 1H), 7.17 (d, 1H), 7.33 (s, 1H), 7.35 (t, 1H), 7.4-7.5 (m, 2H).

### Example 16

### (8S,11S,14S)-14-Amino-N-[(1S)-4-amino-1-({[(1S)-4-amino-1-(2-{[(2S)-2,5-diaminopentyl]-amino}-2-oxoethyl)butyl]amino}carbonyl)butyl]-11-(3-aminopropyl)-17-hydroxy-10,13-dioxo-9,12-diazatricyclo[14.3.1.1^{2,6}]henicosa-1(20),2(21),3,5,16,18-hexaene-8-carboxamide hexahydrochloride

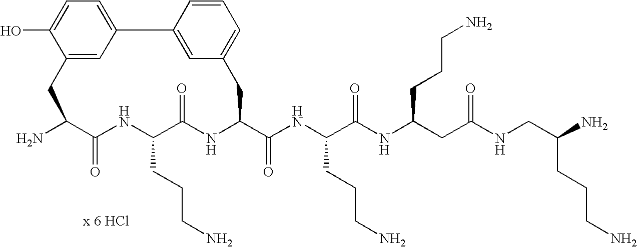

At 0° C., 0.3 ml of a 4N hydrogen chloride-dioxane solution are added to a solution of 28 mg (0.02 mmol) of the compound from Example 140A in 1 ml of dioxane. After 3 h at RT, the reaction solution is concentrated in vacuo and coevaporated several times with dichloromethane. The remaining solid is dried to constant weight under high vacuum.

Yield: 20 mg (99% of theory)

MS (ESI): m/z=782 (M-6HCl+H)⁺.

¹H-NMR (400 MHz, D₂O): δ=1.4-1.9 (m, 16H), 2.4 (m_{c}, 1H), 2.54 (m_{c}, 1H), 2.85-3.2 (m, 11H), 3.29 (m_{c}, 1H), 3.39 (m_{c}, 1H), 3.45-3.65 (m, 2H), 4.1-4.25 (m, 2H), 4.47 (m_{c}, 1H), 4.7-4.9 (m, 2H, under D₂O), 6.94 (d, 1H), 6.99 (s, 1H), 7.17 (d, 1H), 7.33 (s, 1H), 7.35 (t, 1H), 7.4-7.5 (m, 2H).

### Example 17

### (8S,11S,14S)-14-Amino-N-[(1S)-4-amino-1-({[(1S)-4-amino-1-(2-{[(2S)-2,5-diaminopentyl]-amino}-2-oxoethyl)butyl]amino}carbonyl)butyl]-11-[(2R)-3-amino-2-hydroxypropyl]-17-hydroxy-10,13-dioxo-9,12-diazatricyclo[14.3.1.1^{2,6}]henicosa-1(20),2(21),3,5,16,18-hexaene-8-carboxamide hexahydrochloride

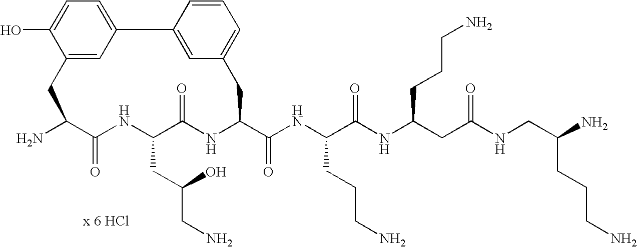

At 0° C., 0.39 ml of a 4N hydrogen chloride-dioxane solution are added to a solution of 36 mg (0.026 mmol) of the compound from Example 141A in 1 ml of dioxane. After 3 h at RT, the reaction solution is concentrated in vacuo and coevaporated several times with dichloromethane. The remaining solid is dried to constant weight under high vacuum.

Yield: 26 mg (99% of theory)

MS (ESI): m/z=798 (M-6HCl+H)⁺.

¹H-NMR (400 MHz, D₂O): δ=1.4-2.05 (m, 14H), 2.41 (m_{c}, 1H), 2.54 (m_{c}, 1H), 2.85-3.2 (m, 11H), 3.29 (m_{c}, 1H), 3.39 (m_{c}, 1H), 3.45-3.65 (m, 2H), 3.85 (m_{c}, 1H), 4.1-4.25 (m, 2H), 4.45 (m_{c}, 1H), 4.7-4.9 (m, 2H, under D₂O), 6.95 (d, 1H), 7.0 (s, 1H), 7.17 (d, 1H), 7.29-7.6 (m, 4H).

### Example 18

### N⁵—(N²-{[(8S,11S,14S)-14-Amino-1-(3-aminopropyl)-17-hydroxy-10,13-dioxo-9,12-diazatricyclo[14.3.1.1^{2,6}]henicosa-1(20),2(21),3,5,16,18-hexaen-8-yl]carbonyl}-L-ornithyl)-N-(2-aminoethyl)-L-ornithinamide pentahydrochloride

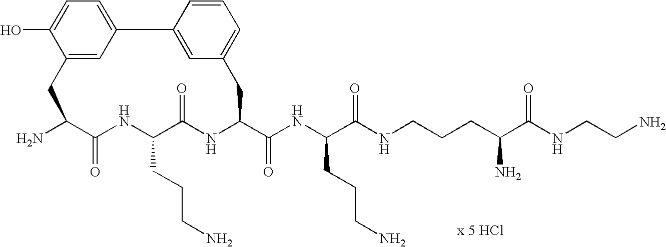

At 0° C., 0.58 ml of a 4N hydrogen chloride-dioxane solution are added to a solution of 47 mg (0.039 mmol) of the compound from Example 142A in 1 ml of dioxane. After 3 h at RT, the reaction solution is concentrated in vacuo and coevaporated several times with dichloromethane. The remaining solid is dried to constant weight under high vacuum.

Yield: 34 mg (99% of theory)

MS (ESI): m/z=711 (M-5HCl+H)⁺.

¹H-NMR (400 MHz, D₂O): δ=1.45-1.95 (m, 12H), 2.9-3.25 (m, 10H), 3.38 (m_{c}, 1H), 3.5-3.7 (m, 2H), 3.96 (m_{c}, 1H), 4.26 (m_{c}, 1H), 4.47 (m_{c}, 1H), 4.7-4.9 (m, 2H, under D₂O), 6.94 (d, 1H), 6.99 (s, 1H), 7.17 (d, 1H), 7.33 (s, 1H), 7.35 (t, 1H), 7.4-7.5 (m, 2H).

### Example 19

### (8S,11S,14S)-14-Amino-N-[(1S)-4-amino-1-(2-{[(2S)-2,5-diaminopentyl]amino}-2-oxoethyl)butyl]-11-(3-aminopropyl)-17-hydroxy-10,13-dioxo-9,12-diazatricyclo[14.3.1.1^{2,6}]henicosa-1 (20),2(21),3,5,16,18-hexaene-8-carboxamide penta(hydrotrifluoroacetate)

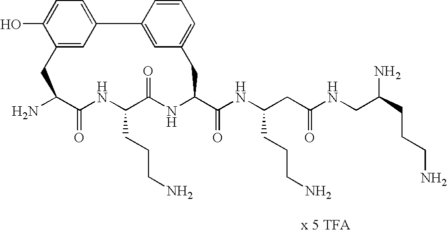

At 0° C., 0.19 ml of a 4N hydrogen chloride-dioxane solution are added to a solution of 15 mg (0.013 mmol) of the compound from Example 143A in 1 ml of dioxane. After 3 h at RT, the reaction solution is concentrated in vacuo and coevaporated several times with dichloromethane. The remaining solid is dried to constant weight under high vacuum. The crude product is converted by preparative HPLC (Reprosil ODS-A, mobile phase acetonitrile/0.2% aqueous trifluoroacetic acid 5:95→95:5) into the tetra(hydrotrifluoroacetate).

Yield: 5.4 mg (34% of theory)

MS (ESI): m/z=668 (M-5TFA+H)⁺.

¹H-NMR (400 MHz, D₂O): δ=1.4-1.9 (m, 12H), 2.39 (m_{c}, 1H), 2.57 (m_{c}, 1H), 2.83-3.17 (m, 9H), 3.32 (m_{c}, 1H), 3.41 (m_{c}, 1H), 3.5-3.7 (m, 2H), 4.21 (m_{c}, 1H), 4.46 (m_{c}, 1H), 4.7-4.9 (m, 2H, under D₂O), 6.94 (d, 1H), 6.98 (s, 1H), 7.11 (d, 1H), 7.32 (s, 1H), 7.35 (t, 1H), 7.44-7.55 (m, 2H).

### Example 20

### (8S,11S,14S)-14-Amino-N-(1-(2-aminoethyl)-3-{[(2S)-2,5-diaminopentyl]amino}-3-oxopropyl)-11-(3-aminopropyl)-17-hydroxy-10,13-dioxo-9,12-diazatricyclo[14.3.1.1^{2,6}]henicosa-1(20),2(21),3,5,16,18-hexaene-8-carboxamide penta(hydrotrifluoroacetate)

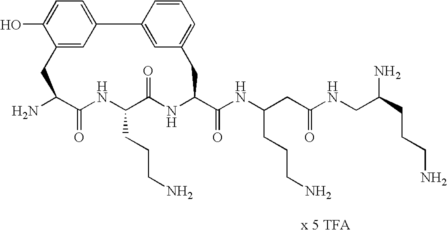

At 0° C., 0.19 ml of a 4N hydrogen chloride-dioxane solution are added to a solution of 14.8 mg (0.013 mmol) of the compound from Example 144A in 1 ml of dioxane. After 3 h at RT, the reaction solution is concentrated in vacuo and coevaporated several times with dichloromethane. The remaining solid is dried to constant weight under high vacuum. The crude product is converted by preparative HPLC (Reprosil ODS-A, mobile phase acetonitrile/0.2% aqueous trifluoroacetic acid 5:95→95:5) into the tetra(hydrotrifluoroacetate).

Yield: 8.9 mg (57% of theory)

MS (ESI): m/z=654 (M-5TFA+H)⁺.

¹H-NMR (400 MHz, D₂O): δ=1.5-2.0 (m, 10H), 2.4-2.65 (m, 2H), 2.85-3.2 (m, 9H), 3.25-3.47 (m, 2H), 3.53-3.68 (m, 2H), 4.27 (m_{c}, 1H), 4.46 (m_{c}, 1H), 4.7-4.9 (m, 2H, under D₂O), 6.9-7.0 (m, 2H), 7.05-7.15 (m, 1H), 7.3-7.4 (m, 2H), 7.42-7.52 (m, 2H).

### Example 21

### (8S,11S,14S)-14-Amino-N-[(1S)-4-amino-1-(2-{[(2S)-2,5-diaminopentyl]amino}-2-oxoethyl)butyl]-11-[(2R)-3-amino-2-hydroxypropyl]-17-hydroxy-9-methyl-10,13-dioxo-9,12-diazatricyclo[14.3.1.1^{2,6}]henicosa-1(20),2(21),3,5,16,18-hexaene-8-carboxamide pentahydrochloride

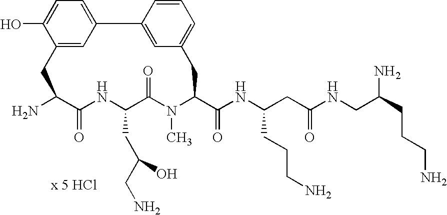

At 0° C., 0.161 ml of a 4N hydrogen chloride-dioxane solution are added to a solution of 12.9 mg (0.011 mmol) of the compound from Example 118A in 1 ml of dioxane. After 3 h at RT, the reaction solution is concentrated in vacuo and coevaporated several times with dichloromethane. The remaining solid is dried to constant weight under high vacuum.

Yield: 9 mg (95% of theory)

MS (ESI): m/z=698 (M-5HCl+H)⁺.

The examples listed in the following table are prepared in analogy to the procedure of Example 1, as hydrochloride or hydro(trifluoroacetate) salt according to the respective isolation method.

| Example | Precursor | | |
|---|---|---|---|
| No. | Example | Structure | Analytical Data |
| 22 | 112A | 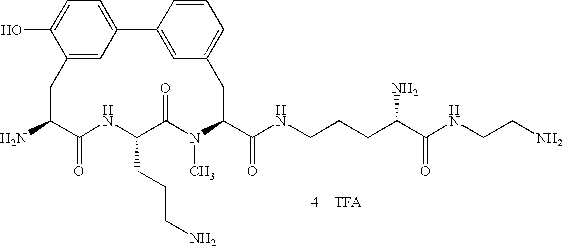 | LC-MS (Method 10):Rₜ = 1.80 minMS (ESI): m/z = 654(M − 4TFA + H)⁺. |
| 23 | 114A | 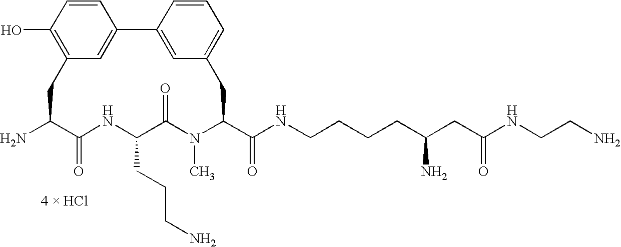 | LC-MS (Method 10):Rₜ = 2.11 minMS (ESI): m/z = 639(M − 4HCl + H)⁺. |
| 24 | 116A | 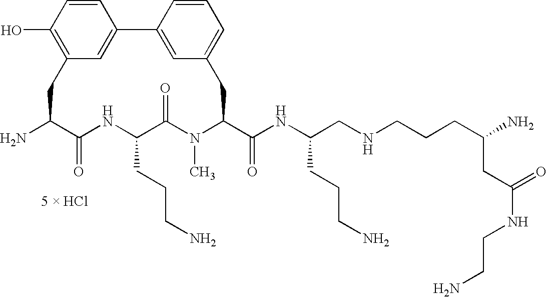 | LC-MS (Method 10):Rₜ = 1.91 minMS (ESI): m/z = 739(M − 5HCl + H)⁺ |
| 25 | 122A | 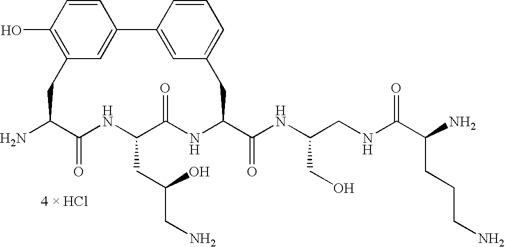 | MS (ESI): m/z = 643(M − 4HCl + H)⁺ |
| 26 | 127A | 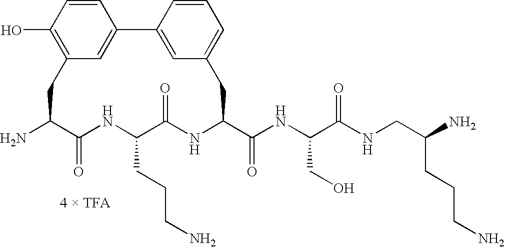 | MS (ESI): m/z = 613(M − 4TFA + H)⁺¹H-NMR (400 MHz,D₂O): δ = 1.5-2.0 (m,8H), 2.85-3.2 (m, 6H),3.3-3.7 (m, 4H), 3.83(m_{c}, 1H), 4.35-4.5 (m,2H), 4.6 (m_{c}, 1H),4.7-4.9 (m, 2H, underD₂O), 6.9-7.0 (m, 2H),7.17 (d, 1H), 7.27-7.4(m, 2H), 7.4-7.5 (m,2H). |
| 27 | 131A | 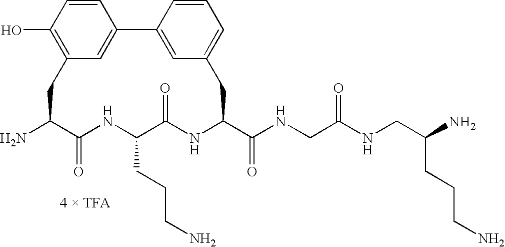 | MS (ESI): m/z = 597(M − 4TFA + H)⁺¹H-NMR (400 MHz,D₂O): δ = 1.5-2.0 (m,8H), 2.9-3.2 (m, 6H),3.3-3.7 (m, 6H), 3.96(m_{c}, 1H), 4.47 (m_{c},1H), 4.7-4.9 (m, 2H,under D₂O), 6.94 (d,1H), 6.98 (s, 1H), 7.17(d, 1H), 7.31 (s, 1H),7.35 (t, 1H), 7.4-7.5(m, 2H). |
| 28 | 132A | 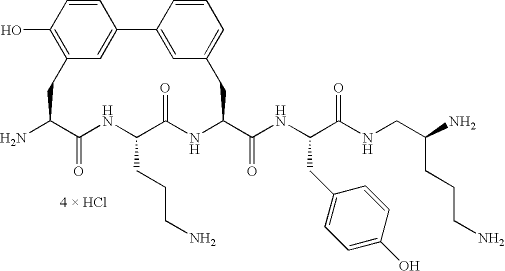 | LC-MS (Method 17):Rₜ = 1.92 minMS (ESI): m/z = 703(M − 4HCl + H)⁺¹H-NMR (400 MHz,D₂O): δ = 1.5-1.8 (m,8H), 2.8-3.1 (m, 9H),3.27 (m_{c}, 1H),3.35-3.45 (m, 2H),3.58 (m_{c}, 1H),4.45-4.55 (m, 2H),4.7-4.9 (m, 2H, underD₂O), 6.7-6.8 (m, 2H),6.9-7.0 (m, 2H),7.05-7.2 (m, 3H), 7.27(s, 1H), 7.34 (t, 1H),7.36-7.46 (m, 2H). |
| 29 | 119A | 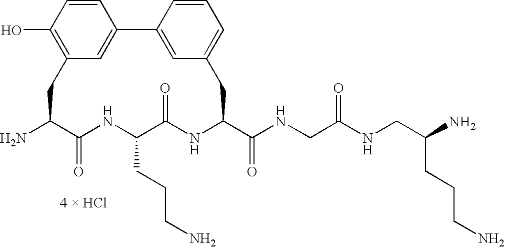 | MS (ESI): m/z = 597(M − 4HCl + H)⁺¹H-NMR (400 MHz,D₂O): δ = 1.55-1.95(m, 8H), 2.85-3.18 (m,7H), 3.2-3.7 (m, 5H),3.95 (m_{c}, 1H), 4.45(m_{c}, 1H), 4.7-4.9 (m,2H, under D₂O), 6.94(d, 1H), 6.98 (s, 1H),7.17 (d, 1H), 7.31 (s,1H), 7.35 (t, 1H),7.4-7.5 (m, 2H). |
| 30 | 123A | 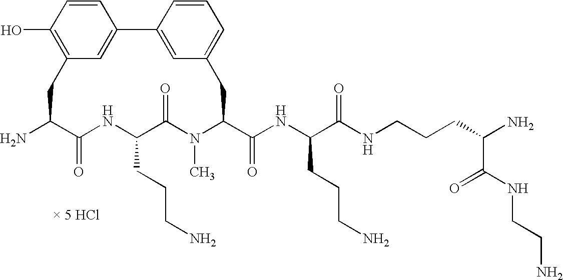 | LC-MS (Method 10):Rₜ = 1.77 minMS (ESI): m/z = 725(M − 5HCl + H)⁺ |
| 31 | 124A | 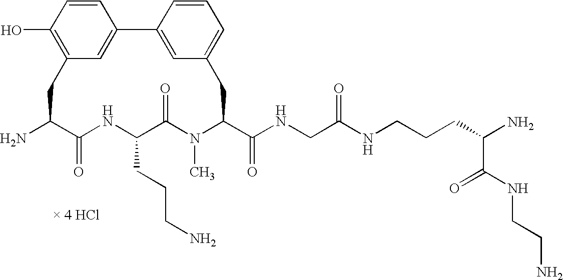 | LC-MS (Method 10):Rₜ = 1.95 minMS (ESI): m/z = 668(M − 4HCl + H)⁺ |
| 32 | 125A | 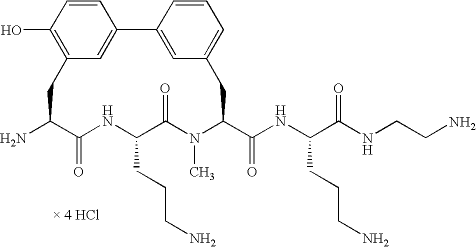 | LC-MS (Method 10):Rₜ = 1.92 minMS (ESI): m/z = 611(M − 4HCl + H)⁺ |
| 33 | 126A | 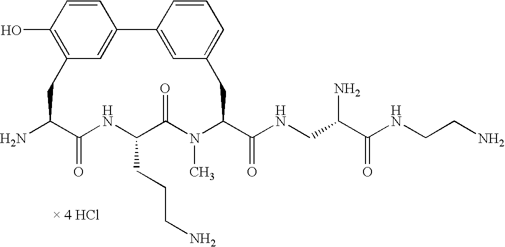 | LC-MS (Method 10):Rₜ = 1.81 minMS (ESI): m/z = 583(M − 4HCl + H)⁺ |
| 34 | 145A | 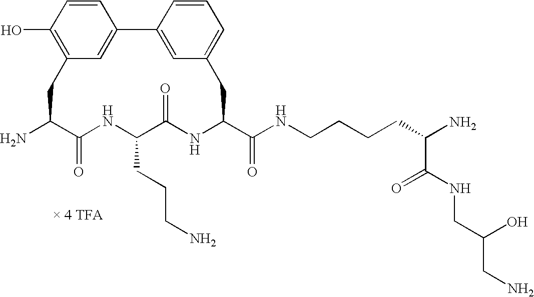 | MS (ESI): m/z = 641(M − 4TFA + H)⁺ |
| 35 | 146A | 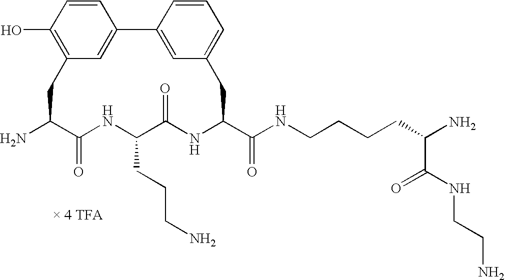 | MS (ESI): m/z = 611(M − 4TFA + H)⁺ |
| 36 | 147A | 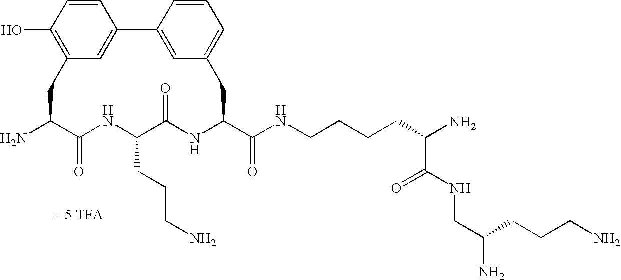 | MS (ESI): m/z = 668(M − 5TFA + H)⁺ |
| 37 | 148A | 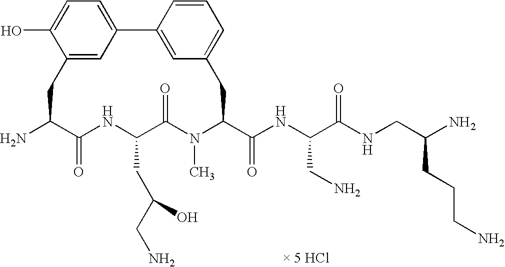 | MS (ESI): m/z = 655(M − 5HCl + H)⁺. |
| 38 | 149A | 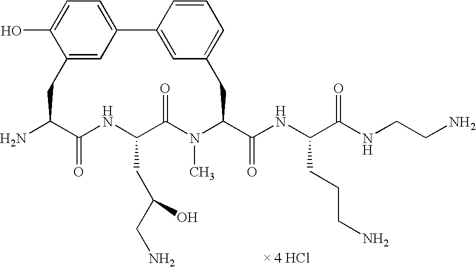 | MS (ESI): m/z = 627(M − 4HCl + H)⁺. |

Examples 39 to 93 listed in the following table are prepared in analogy to the procedure of Example 1, as hydrochloride or hydro(trifluoroacetate) salt according to the respective isolation method.

| Example | Precursor | | |
|---|---|---|---|
| No. | Example | Structure | Analytical Data |
| 39 | 227A | 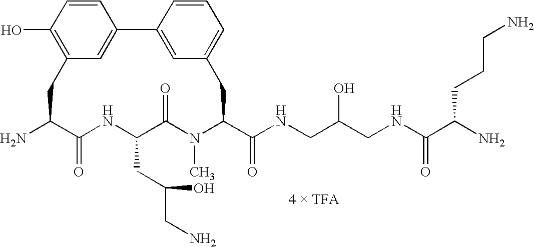 | LC-MS (Method 2):Rₜ = 0.25 minMS (ESI): m/z = 657(M − 4TFA + H)⁺. |
| 40 | 228A | 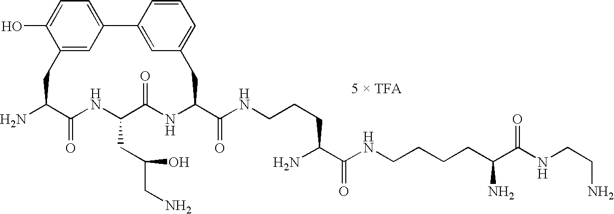 | LC-MS (Method 10):Rₜ = 1.08 minMS (ESI): m/z = 741(M − 5TFA + H)⁺. |
| 41 | 229A | 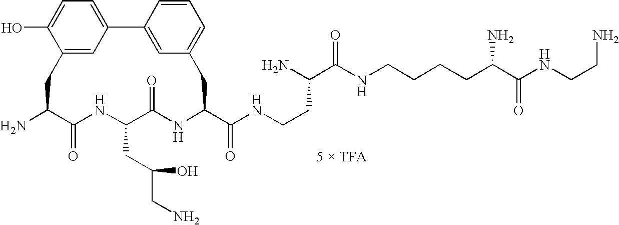 | LC-MS (Method 10):Rₜ = 0.86 minMS (ESI): m/z = 727(M − 5TFA + H)⁺. |
| 42 | 230A | 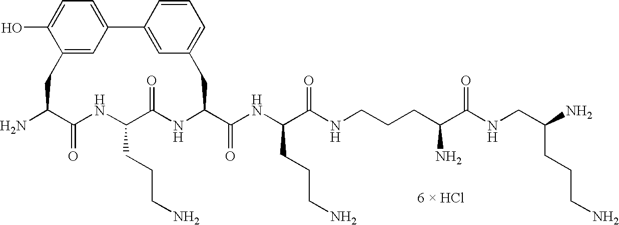 | LC-MS (Method 1):Rₜ = 0.3 minMS (ESI): m/z = 768(M − 6HCl + H)⁺¹H-NMR (400 MHz,D₂O): δ = 1.5-1.9 (m,16H), 2.9-3.3 (m, 9H),3.4-3.8 (m, 6H), 4.0(m_{c}, 1H), 4.26 (m_{c},1H), 4.47 (m_{c}, 1H),4.7-4.9 (m, 2H, underD₂O), 6.95 (d, 1H),6.99 (s, 1H), 7.17 (d,1H), 7.31 (s, 1H), 7.35(t, 1H), 7.4-7.5 (m,2H). |
| 43 | 231A | 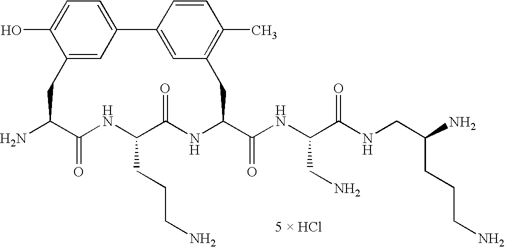 | LC-MS (Method 10):Rₜ = 0.46 minMS (ESI): m/z = 640(M − 5HCl + H)⁺. |
| 44 | 232A | 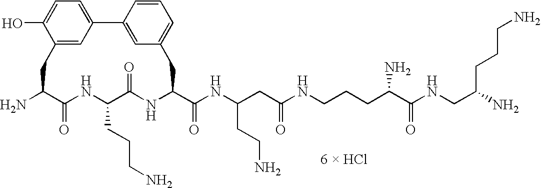 | LC-MS (Method 1):Rₜ = 0.31 minMS (ESI): m/z = 768(M − 6HCl + H)⁺. |
| 45 | 233A | 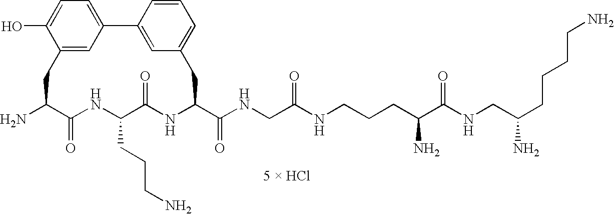 | LC-MS (Method 2):Rₜ = 0.26 minMS (ESI): m/z = 711(M − 5HCl + H)⁺. |
| 46 | 234A | 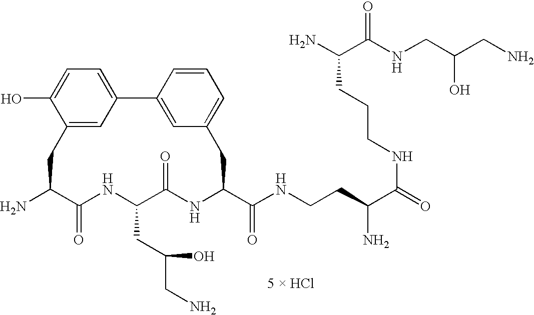 | LC-MS (Method 2):Rₜ = 0.28 minMS (ESI): m/z = 743(M − 5HCl + H)⁺. |
| 47 | 235A | 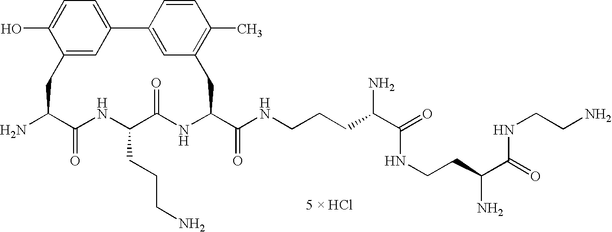 | LC-MS (Method 1):Rₜ = 0.30 minMS (ESI): m/z = 711(M − 5HCl + H)⁺. |
| 48 | 236A | 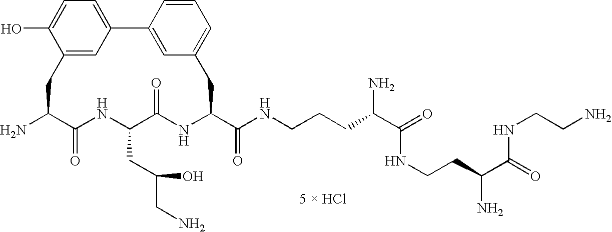 | LC-MS (Method 1):Rₜ = 0.31 minMS (ESI): m/z = 713(M − 5HCl + H)⁺. |
| 49 | 237A | 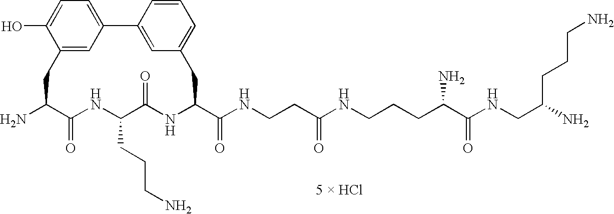 | LC-MS (Method 1):Rₜ = 0.31 minMS (ESI): m/z = 725(M − 5HCl + H)⁺. |
| 50 | 238A | 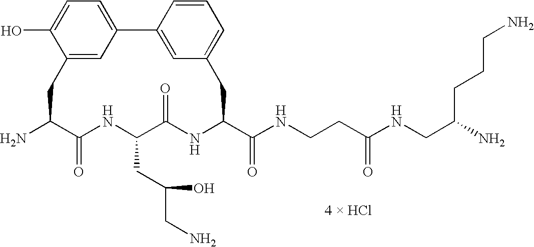 | LC-MS (Method 1):Rₜ = 0.23 minMS (ESI): m/z = 627(M − 4HCl + H)⁺. |
| 51 | 239A | 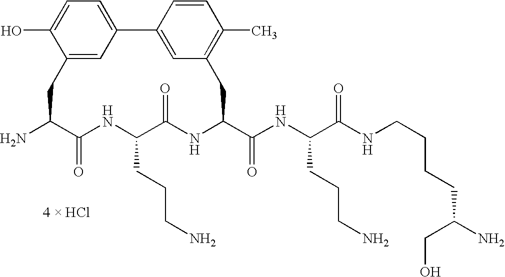 | LC-MS (Method 10):Rₜ = 1.95 minMS (ESI): m/z = 683(M − 4HCl + H)⁺. |
| 52 | 240A | 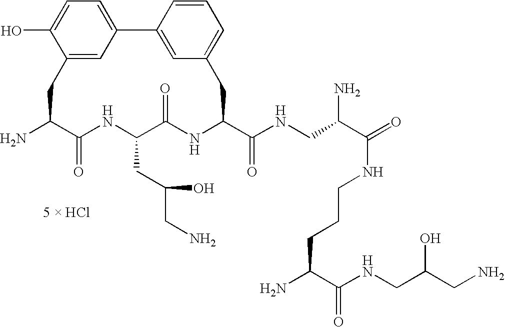 | LC-MS (Method 2):Rₜ = 0.28 minMS (ESI): m/z = 729(M − 5HCl + H)⁺. |
| 53 | 241A | 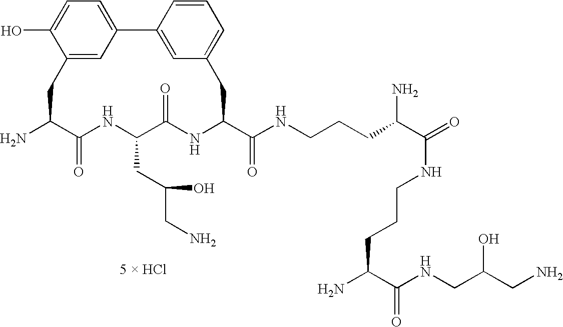 | LC-MS (Method 3):Rₜ = 0.26 minMS (ESI): m/z = 757(M − 5HCl + H)⁺. |
| 54 | 242A | 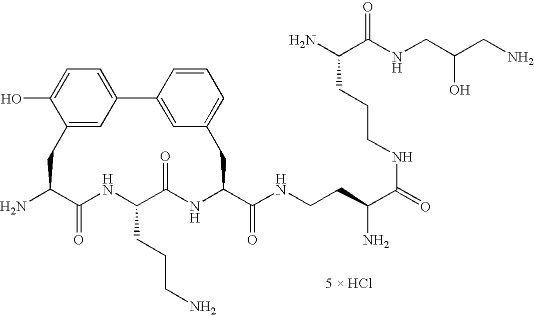 | LC-MS (Method 2):Rₜ = 0.28 minMS (ESI): m/z = 727(M − 5HCl + H)⁺. |
| 55 | 243A | 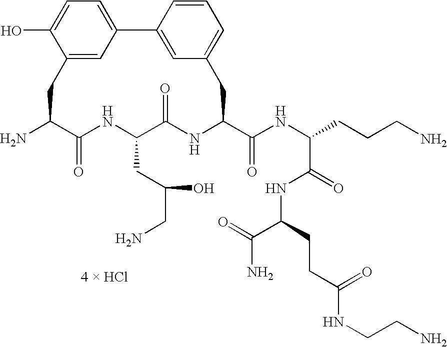 | LC-MS (Method 10):Rₜ = 1.96 minMS (ESI): m/z = 741(M − 4HCl + H)⁺.¹H-NMR (400 MHz,D₂O): δ = 1.6-2.15(m, 8H), 2.3 (m, 2H),2.9-3.3 (m, 10H),3.4-3.8 (m, 4H), 3.85(m_{c}, 1H), 4.22 (m_{c},1H), 4.35 (m_{c}, 1H),4.43 (m_{c}, 1H), 4.7-4.9(m, 2H, under D₂O),6.94 (d, 1H), 6.98 (s,1H), 7.17 (d, 1H),7.32 (s, 1H), 7.35 (t,1H), 7.4-7.5 (m, 2H). |
| 56 | 244A | 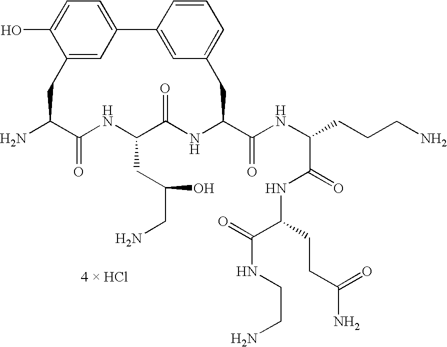 | LC-MS (Method 10):Rₜ = 1.86 minMS (ESI): m/z = 741(M − 4HCl + H)⁺. |
| 57 | 245A | 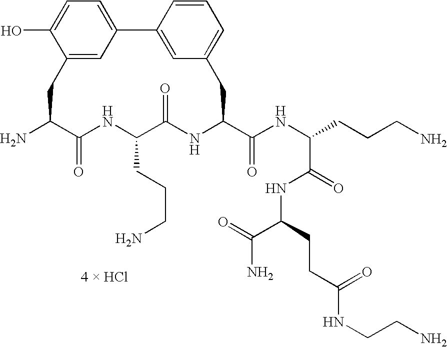 | LC-MS (Method 10):Rₜ = 1.96 minMS (ESI): m/z = 739(M − 4HCl + H)⁺. |
| 58 | 246A | 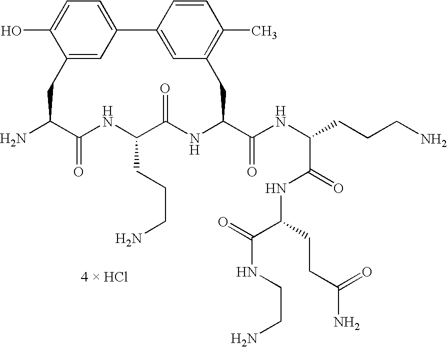 | LC-MS (Method 10):Rₜ = 2.10 minMS (ESI): m/z = 739(M − 4HCl + H)⁺. |
| 59 | 247A | 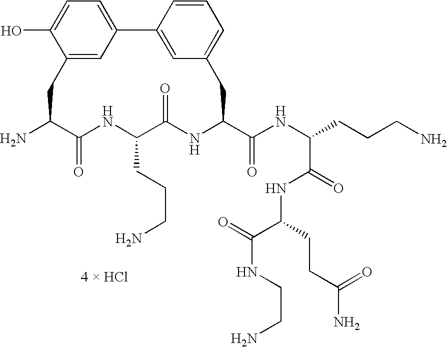 | LC-MS (Method 10):Rₜ = 1.87 minMS (ESI): m/z = 725(M − 4HCl + H)⁺. |
| 60 | 248A | 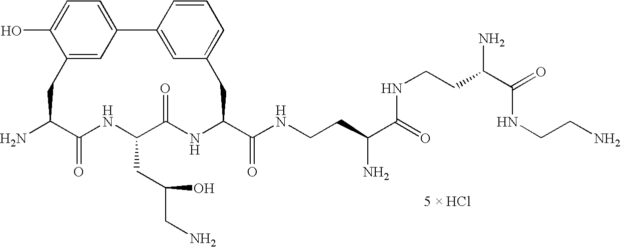 | LC-MS (Method 3):Rₜ = 0.25 minMS (ESI): m/z = 699(M − 5HCl + H)⁺. |
| 61 | 249A | 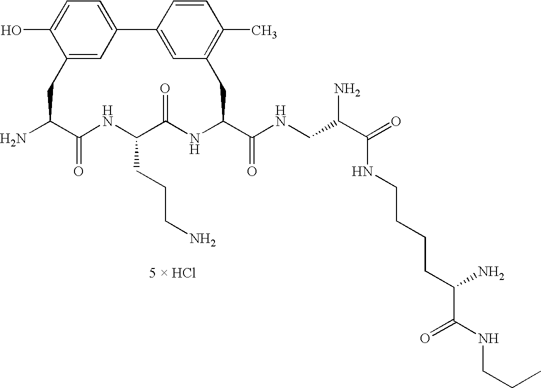 | LC-MS (Method 2):Rₜ = 0.28 minMS (ESI): m/z = 711(M − 5HCl + H)⁺. |
| 62 | 250A | 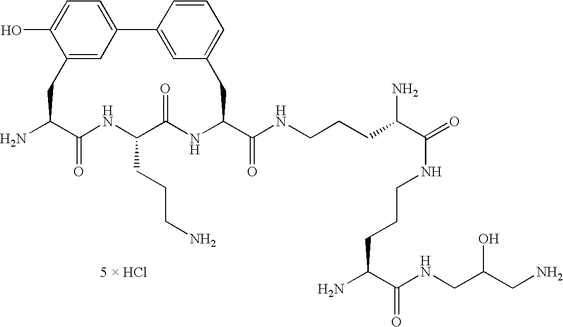 | LC-MS (Method 2):Rₜ = 0.28 minMS (ESI): m/z = 741(M − 5HCl + H)⁺. |
| 63 | 251A | 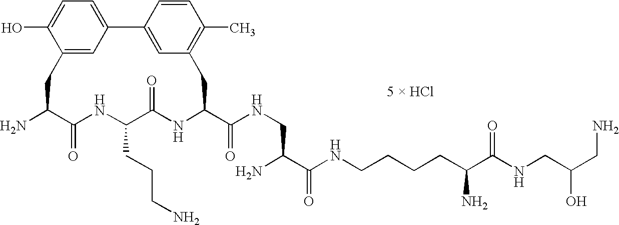 | LC-MS (Method 2):Rₜ = 0.24 minMS (ESI): m/z = 741(M − 5HCl + H)⁺. |
| 64 | 252A | 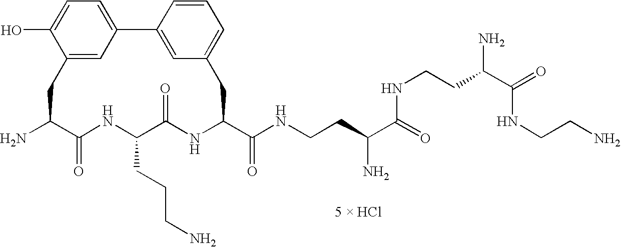 | LC-MS (Method 2):Rₜ = 0.28 minMS (ESI): m/z = 683(M − 5HCl + H)⁺. |
| 65 | 253A | 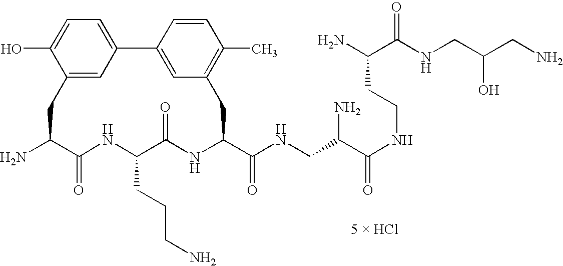 | LC-MS (Method 2):Rₜ = 0.28 minMS (ESI): m/z = 699(M − 5HCl + H)⁺. |
| 66 | 254A | 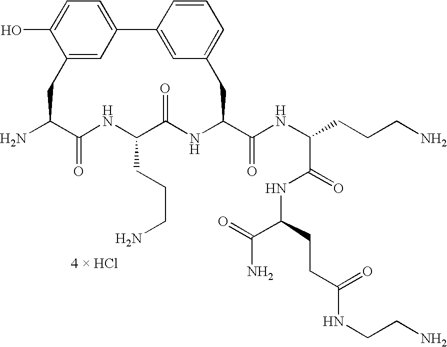 | LC-MS (Method 10):Rₜ = 1.88 minMS (ESI): m/z = 725(M − 4HCl + H)⁺. |
| 67 | 255A | 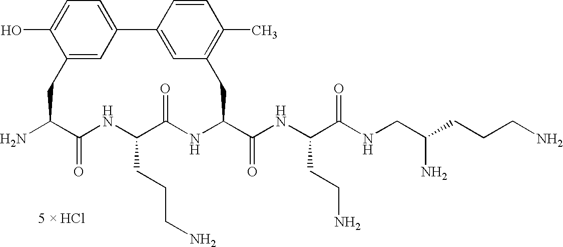 | LC-MS (Method 2):Rₜ = 0.29 minMS (ESI): m/z = 668(M − 5HCl + H)⁺.¹H-NMR (400 MHz,D₂O): δ = 1.55-1.95(m, 12H), 2.24 (s,3H), 2.8-3.2 (m, 9H),3.3-3.7 (m, 4H), 4.33(m_{c}, 1H), 4.46 (m_{c},1H), 4.63 (m_{c}, 1H),4.94 (m_{c}, 1H), 6.94 (d,1H), 7.07 (s, 1H), 7.25(d, 1H), 7.30 (s, 1H),7.45 (d, 1H), 7.55 (d,1H) |
| 68 | 256A | 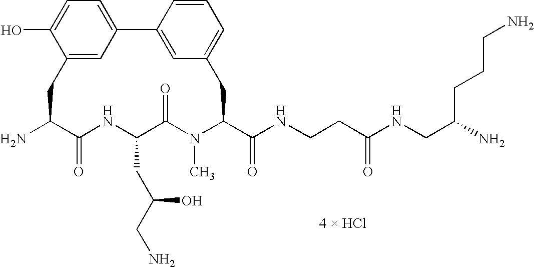 | LC-MS (Method 2):Rₜ = 0.27 minMS (ESI): m/z = 641(M − 4HCl + H)⁺.¹H-NMR (400 MHz,D₂O): δ = 1.55-1.95(m, 6H), 2.49 (m, 2H),2.8-3.8 (m, 13H), 3.96(m_{c}, 1H), 4.46 (m_{c},1H), 5.11 (m_{c}, 1H),5.61 (m_{c}, 1H),6.92-7.02 (m, 2H),7.10 (s, 1H), 7.18 (d,1H), 7.36 (t, 1H), 7.49(d, 1H), 7.55 (d, 1H) |
| 69 | 257A | 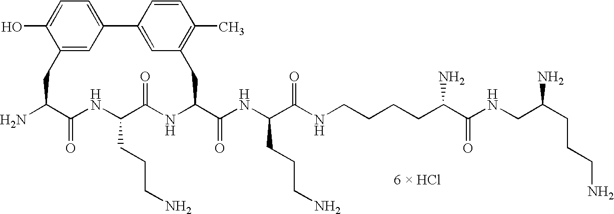 | LC-MS (Method 2):Rₜ = 0.20 minMS (ESI): m/z = 796(M − 6HCl + H)⁺.¹H-NMR (400 MHz,D₂O): δ = 1.3-1.95(m, 18H), 2.23 (s,3H), 2.8-3.8 (m, 17H),3.98 (m_{c}, 1H), 4.26(m_{c}, 1H), 4.46 (m_{c},1H), 4.63 (m_{c}, 1H),4.93 (m_{c}, 1H), 6.94 (d,1H), 7.07 (s, 1H), 7.25(d, 1H), 7.28 (s, 1H),7.44 (td 1H), 7.54 (d,1H). |
| 70 | 258A | 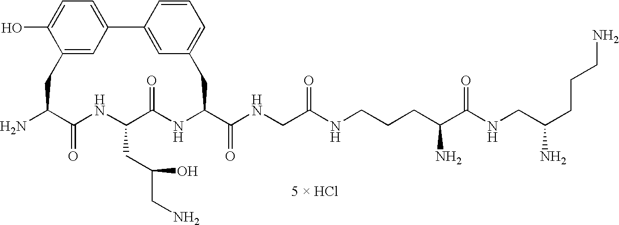 | LC-MS (Method 2):Rₜ = 0.25 minMS (ESI): m/z = 741(M − 5HCl + H)⁺. |
| 71 | 259A | 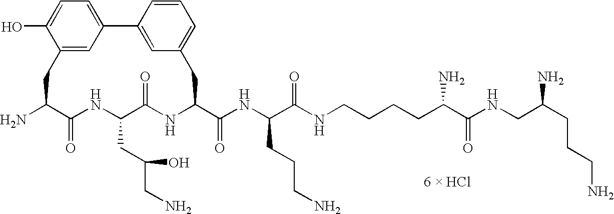 | LC-MS (Method 10):Rₜ = 0.86 minMS (ESI): m/z = 798(M − 6HCl + H)⁺. |
| 72 | 260A | 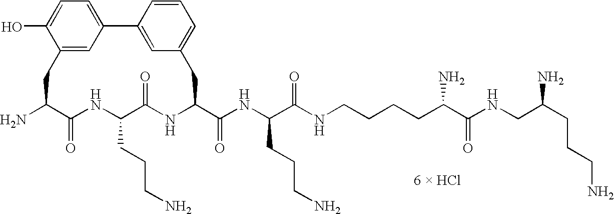 | LC-MS (Method 2):Rₜ = 0.15 minMS (ESI): m/z = 782(M − 6HCl + H)⁺.¹H-NMR (400 MHz,D₂O): δ = 1.3-1.95(m, 18H), 2.8-3.8 (m,17H), 3.97 (m_{c}, 1H),4.26 (m_{c}, 1H), 4.46(m_{c}, 1H), 4.6-4.9 (m,2H, under D₂O), 6.95(d, 1H), 6.99 (s, 1H),7.16 (d, 1H),7.29-7.39 (m, 2H),7.4-7.5 (m, 2H). |
| 73 | 261A | 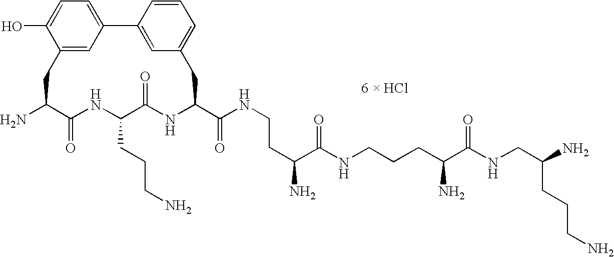 | LC-MS (Method 2):Rₜ = 0.15 minMS (ESI): m/z = 754(M − 6HCl + H)⁺. |
| 74 | 262A | 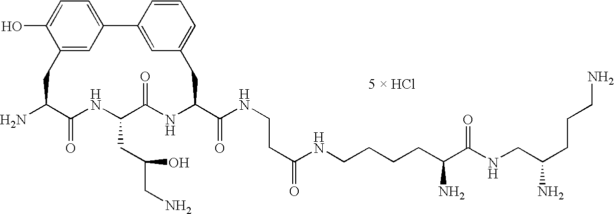 | LC-MS (Method 2):Rₜ = 0.2 minMS (ESI): m/z = 755(M − 5HCl + H)⁺. |
| 75 | 263A | 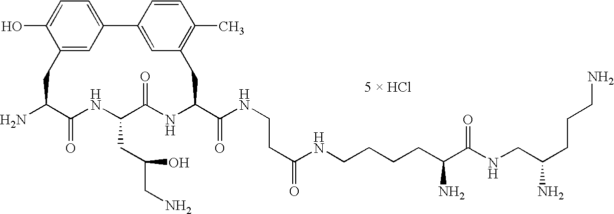 | LC-MS (Method 2):Rₜ = 0.2 minMS (ESI): m/z = 753(M − 5HCl + H)⁺. |
| 76 | 264A | 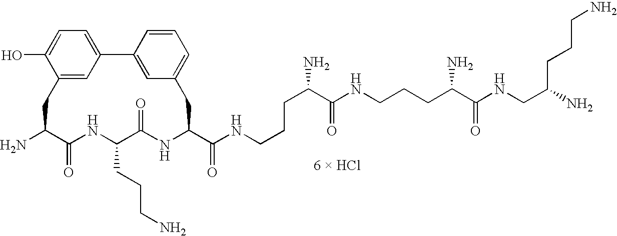 | LC-MS (Method 2):Rₜ = 0.2 minMS (ESI): m/z = 768(M − 6HCl + H)⁺. |
| 77 | 265A | 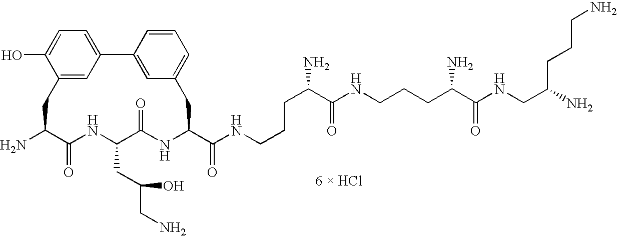 | LC-MS (Method 2):Rₜ = 0.2 minMS (ESI): m/z = 784(M − 6HCl + H)⁺. |
| 78 | 266A | 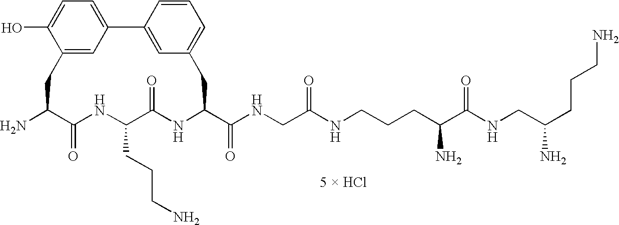 | LC-MS (Method 2):Rₜ = 0.26 minMS (ESI): m/z = 725(M − 5HCl + H)⁺. |
| 79 | 280A | 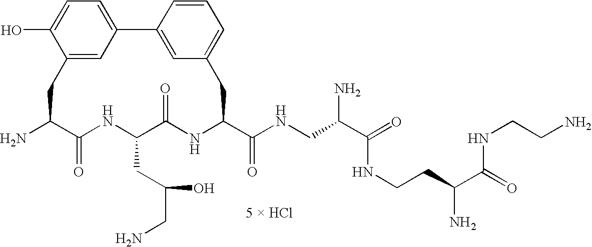 | LC-MS (Method 2):Rₜ = 0.2 minMS (ESI): m/z = 685(M − 5HCl + H)⁺. |
| 80 | 281A | 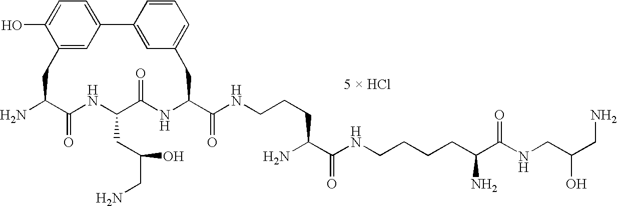 | LC-MS (Method 2):Rₜ = 0.2 minMS (ESI): m/z = 771(M − 5HCl + H)⁺. |
| 81 | 269A | 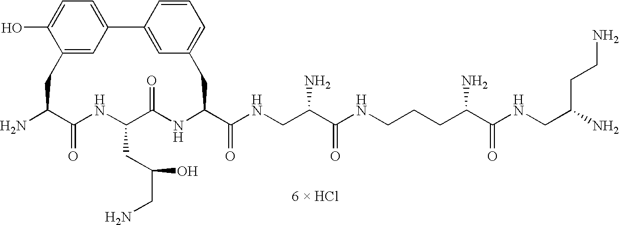 | LC-MS (Method 2):Rₜ = 0.2 minMS (ESI): m/z = 756(M − 6HCl + H)⁺. |
| 82 | 270A | 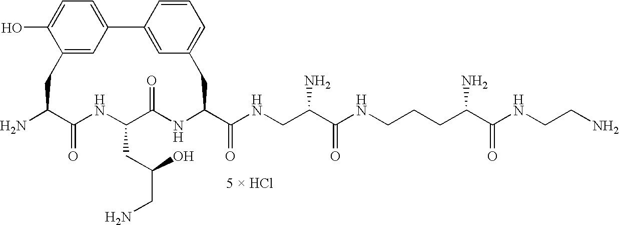 | LC-MS (Method 2):Rₜ = 0.2 minMS (ESI): m/z = 699(M − 5HCl + H)⁺. |
| 83 | 271A | 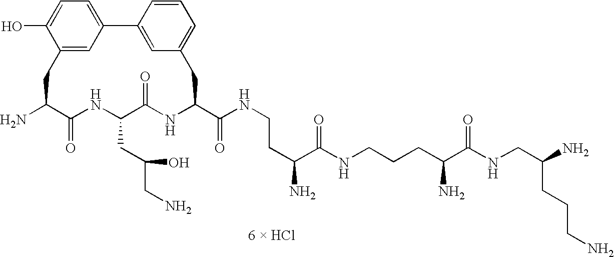 | LC-MS (Method 2):Rₜ = 0.2 minMS (ESI): m/z = 770(M − 6HCl + H)⁺. |
| 84 | 279A | 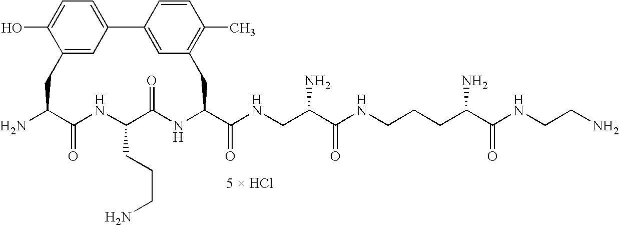 | LC-MS (Method 2):Rₜ = 0.2 minMS (ESI): m/z = 739(M − 5HCl + H)⁺. |
| 85 | 273A | 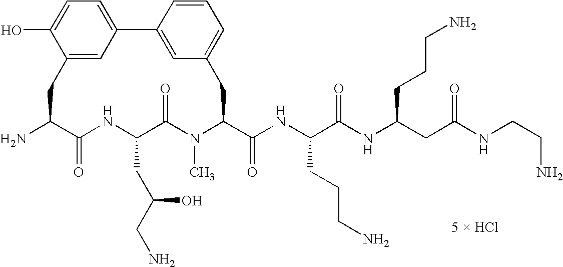 | LC-MS (Method 2):Rₜ = 0.26 minMS (ESI): m/z = 755(M − 5HCl + H)⁺. |
| 86 | 274A | 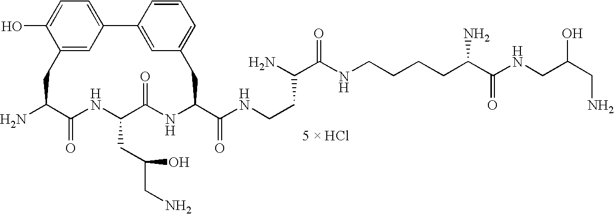 | LC-MS (Method 2):Rₜ = 0.2 minMS (ESI): m/z = 757(M − 5HCl + H)⁺. |
| 87 | 275A | 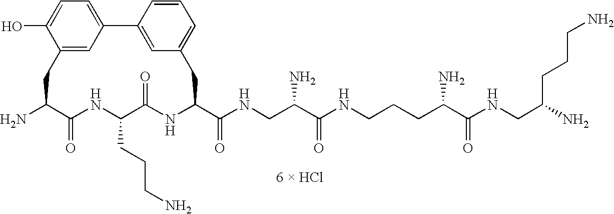 | LC-MS (Method 2):Rₜ = 0.2 minMS (ESI): m/z = 740(M − 6HCl + H)⁺. |
| 88 | 278A | 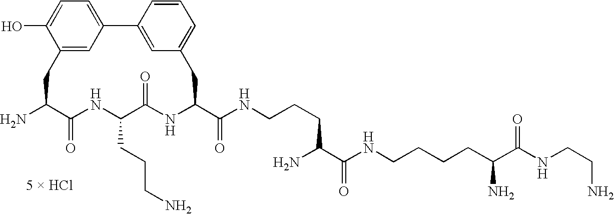 | LC-MS (Method 2):Rₜ = 0.2 minMS (ESI): m/z = 725(M − 5HCl + H)⁺. |
| 89 | 277A | 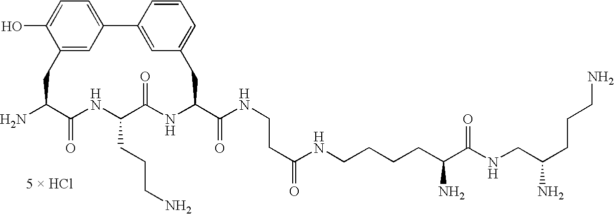 | LC-MS (Method 2):Rₜ = 0.2 minMS (ESI): m/z = 739(M − 5HCl + H)⁺. |

### Assessment of the Physiological Activity

### Abbreviations Used:

AMP adenosine monophosphate

ATP adenosine triphosphate

BHI medium brain heart infusion medium

CoA coenzyme A

DMSO dimethyl sulfoxide

DTT dithiothreitol

EDTA ethylenediaminetetraacetic acid

KCl potassium chloride

KH₂PO₄ potassium dihydrogen phosphate

MgSO₄ magnesium sulfate

MIC minimum inhibitory concentration

MTP microtiter plate

NaCl sodium chloride

Na₂HPO₄ disodium hydrogen phosphate

NH₄Cl ammonium chloride

NTP nucleotide triphosphate

PBS phosphate-buffered saline

PCR polymerase chain reaction

PEG polyethylene glycol

PEP phosphoenolpyruvate

Tris tris[hydroxymethyl)aminomethane

The in vitro effect of the compounds of the invention can be shown in the following assays:

In Vitro Transcription-Translation with E. Coli Extracts

In order to prepare an S30 extract logarithmically growing Escherichia coli MRE 600 (M. Müller; Freiburg University) are harvested, washed and employed it as described for the in vitro transcription-translation test (Müller, M. and Blobel, G. Proc Natl Acad Sci USA (1984) 81, pp. 7421-7425).

1 μl of cAMP (11.25 mg/ml) are additionally added per 50 μl of reaction mix to the reaction mix of the in vitro transcription-translation tests. The test mixture amounts to 105 μl, with 5 μl of the substance to be tested being provided in 5% DMSO. 1 μg/100 μl of mixture of the plasmid pBESTLuc (Promega, Germany) are used as transcription template. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are added, and the resulting bioluminescence is measured in a luminometer for 1 minute. The concentration of an inhibitor which leads to a 50% inhibition of the translation of firefly luciferase is reported as the IC₅₀.

In Vitro Transcription-Translation with S. aureus Extracts

Construction of an S. aureus Luciferase Reporter Plasmid

For the construction of a reporter plasmid which can be used in an in vitro transcription-translation assay from S. aureus the plasmid pBESTluc (Promega Corporation, USA) is used. The E. coli tac promoter present in this plasmid in front of the firefly luciferase is replaced by the capA1 promoter with corresponding Shine-Dalgarno sequence from S. aureus. The primers CAPFor 5′-CGGCCAAGCTTACTCGGATCCAGAGTTTGCAAAATATACAG-GGGATTATATATAATGGAAAACAAGAAAGGAAAATAGGAGGTTTATATGGAAGAC GCCA-3′ and CAPRev 5′-GTCATCGTCGGGAAGACCTG-3′ are used for this. The primer CAPFor contains the capA1 promoter, the ribosome binding site and the 5′ region of the luciferase gene. After PCR using pBESTluc as template it is possible to isolate a PCR product which contains the firefly luciferase gene with the fused capA1 promoter. This is, after restriction with ClaI and HindIII, ligated into the vector pBESTluc which has likewise been digested with ClaI and HindIII. The resulting plasmid pla can be replicated in E. coli and be used as template in the S. aureus in vitro transcription-translation test.

Preparation of S30 Extracts from S. aureus

Six litres of BHI medium are inoculated with a 250 ml overnight culture of an S. aureus Strain and allowed to grow at 37° C. until the OD600 nm is 2-4. The cells are harvested by centrifugation and washed in 500 ml of cold buffer A (10 mM Tris acetate, pH 8.0, 14 mM magnesium acetate, 1 mM DTT, 1 M KCl). After renewed centrifugation, the cells are washed in 250 ml of cold buffer A with 50 mM KCl, and the resulting pellets are frozen at −20° C. for 60 min. The pellets are thawed on ice in 30 to 60 min and taken up to a total volume of 99 ml in buffer B (10 mM Tris acetate, pH 8.0, 20 mM magnesium acetate, 1 mM DTT, 50 mM KCl). 1.5 ml portions of lysostaphin (0.8 mg/ml) in buffer B are provided in 3 precooled centrifuge cups and each mixed with 33 ml of the cell suspension. The samples are incubated at 37° C., shaking occasionally, for 45 to 60 min, before 150 μl of a 0.5 M DTT solution are added. The lysed cells are centrifuged at 30 000×g and 4° C. for 30 min. The cell pellet is taken up in buffer B and then centrifuged again under the same conditions, and the collected supernatants are combined. The supernatants are centrifuged again under the same conditions, and 0.25 volumes of buffer C (670 mM Tris acetate, pH 8.0, 20 mM magnesium acetate, 7 mM Na₃ phosphoenolpyruvate, 7 mM DTT, 5.5 mM ATP, 70 μM amino acids (complete from Promega), 75 μg of pyruvate kinase (Sigma, Germany)/ml are added to the upper ⅔ of the supernatant. The samples are incubated at 37° C. for 30 min. The supernatants are dialysed against 2 l of dialysis buffer (10 mM Tris acetate, pH 8.0, 14 mM magnesium acetate, 1 mM DTT, 60 mM potassium acetate) in a dialysis tube with a 3500 Da cut-off with one buffer change at 4° C. overnight. The dialysate is concentrated to a protein concentration of about 10 mg/ml by

covering the dialysis tube with cold PEG 8000 powder (Sigma, Germany) at 4° C. The S30 extracts can be stored in aliquots at −70° C.

Determination of the IC₅₀ in the S. aureus In Vitro Transcription-Translation Assay

The inhibition of protein biosynthesis of the compounds can be shown in an in vitro transcription-translation assay. The assay is based on the cell-free transcription and translation of firefly luciferase using the reporter plasmid pla as template and cell-free S30 extracts obtained from S. aureus. The activity of the resulting luciferase can be detected by luminescence measurement.

The amount of S30 extract or plasmid pla to be employed must be tested anew for each preparation in order to ensure an optimal concentration in the test. 3 l of the substance to be tested, dissolved in 5% DMSO, are introduced into an MTP. Then 101 of a suitably concentrated plasmid solution pla are added. Then 46 μl of a mixture of 23 g of premix (500 mM potassium acetate, 87.5 mM Tris acetate, pH 8.0, 67.5 mM ammonium acetate, 5 mM DTT, 50 μg of folic acid/ml, 87.5 mg of PEG 8000/ml, 5 mM ATP, 1.25 mM of each NTP, 20 μM of each amino acid, 50 mM PEP (Na₃ Salt), 2.5 mM cAMP, 250 μg of each E. coli tRNA/ml) and 23 g of a suitable amount of S. aureus S30 extract are added and mixed. After incubation at 30° C. for 60 min, 501 of luciferin solution (20 mM tricine, 2.67 mM MgSO₄, 0.1 mM EDTA, 33.3 mM DTT pH 7.8, 270 μM CoA, 470 μM luciferin, 530 μM ATP) are, and the resulting bioluminescence is measured in a luminometer for 1 min. The concentration of an inhibitor which leads to a 50% inhibition of the translation of firefly luciferase is reported as the IC₅₀.

### Determination of the Minimum Inhibitory Concentration (CLSI Standard)

The minimum inhibitory concentration (MIC) is the minimum concentration of an antibiotic with which the growth of a test microbe is inhibited over 18-24 h. The inhibitor concentration can in these cases be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The test substances are thereby initially provided in 1:2 dilution series into 96-well round-bottom microtiter plates (Greiner) double-concentrated in 50 μl of test medium. The aerobically growing test microbes (e.g. staphylococci and enterococci), which are incubated overnight on Columbia blood agar plates (Becton-Dickinson), are, after resuspension in 0.9% NaCl, adjusted to a microbe count of about 5×10⁷ microbes/ml and then diluted 1:150 in cation-adjusted MH medium (test medium). 50 μL of this suspension are pipetted onto the test preparations provided in the microtiter plates. The cultures are incubated at 37° C. for 18-24 hours. For microaerophilically growing microbes (e.g. streptococci), 2% lysed horse blood in the final concentration is added to the medium and the cultures are incubated in the presence of 5% CO₂. The lowest substance concentration in each case at which no visible bacterial growth occurs any longer is defined as the MIC and is reported in μg/ml.

Determination of the Minimum Inhibitory Concentration (MIC)

The minimum inhibitory concentration (MIC) is the minimum concentration of an antibiotic with which the growth of a test microbe is inhibited over 18-24 h. The inhibitor concentration can in these cases be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The MIC of the compounds of the invention is determined in the liquid dilution test on the 96-well microtiter plate scale. The bacterial microbes are cultivated in a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the exception of phenylalanine; [H.-P. Kroll; unpublished]) with the addition of 0.4% BH broth (test medium). In the case of Enterococcus faecium L4001, heat-inactivated fetal calf serum (FCS; GibcoBRL, Germany) is added to the test medium in a final concentration of 10%. Overnight cultures of the test microbes are diluted to an OD₅₇₈ of 0.001 (to 0.01 in the case of enterococci) in fresh test medium, and incubated 1:1 with dilutions of the test substances (1:2 dilution steps) in test medium (2001 final volume). The cultures are incubated at 37° C. for 18-24 hours; enterococci in the presence of 5% CO₂.

The lowest substance concentration in each case at which no visible bacterial growth occurs any longer is defined as the MIC.

Alternative Method for Determining the Minimum Inhibitory Concentration (MIC)

The minimum inhibitory concentration (MIC) is the minimum concentration of an antibiotic with which the growth of a test microbe is inhibited over 18-24 h. The inhibitor concentration can in these cases be determined by standard microbiological methods with modified medium in an agar dilution test (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The bacterial microbes are cultivated on 1.5% agar plates which contain 20% defibrinated horse blood. The test microbes, which are incubated overnight on Columbia blood agar plates (Becton-Dickinson), are diluted in PBS, adjusted to a microbe count of about 5×10⁵ microbes/ml and placed dropwise (1-3 μl) on test plates. The test substances comprise different dilutions of the test substances (1:2 dilution steps). The cultures are incubated at 37° C. in the presence of 5% CO₂ for 18-24 hours.

The lowest substance concentration in each case at which no visible bacterial growth occurs any longer is defined as the MIC and is reported in μg/ml.

TABLE A
(with comparative example biphenomycin B)

| | MIC | MIC | MIC | IC₅₀ |
|---|---|---|---|---|
| | S. aureus | S. aureus | E. faecium | S. aureus 133 |
| Ex. No. | 133 | T17 | L4001 | Translation |
| 1 | 0.5 | 1.0 | 4.0 | 0.07 |
| 2 | 1.0 | 1.0 | 2.0 | 0.07 |
| 3 | 2.0 | 2.0 | 16.0 | 0.2 |
| 5 | 1.0 | 1.0 | 2.0 | 0.2 |
| 12 | 1.0 | 1.0 | 16.0 | 0.08 |
| 15 | 1.0 | 2.0 | >32 | 0.1 |
| 19 | 1.0 | 1.0 | 16.0 | 0.1 |
| 67 | 1.0 | 1.0 | 16 | 0.1 |
| 68 | 1.0 | 1.0 | 8 | 0.2 |
| Biphenomycin | <0.03 | >32 | 0.5 | 1.5 |
| B | | | | |

Concentration data: MIC in μ/ml; IC₅₀ in μM.

Systemic Infection with S. aureus 133

The suitability of the compounds of the invention for treating bacterial infections can be shown in various animal models. For this purpose, the animals are generally infected with a suitable virulent microbe and then treated with the compound to be tested, which is in a formulation which is adapted to the particular therapy model. The suitability of the compounds of the invention for the treatment of bacterial infections can be demonstrated specifically in a mouse sepsis model after infection with S. aureus.

For this purpose, S. aureus 133 cells are cultured overnight in BH broth (Oxoid, Germany). The overnight culture was diluted 1:100 in fresh BH broth and expanded for 3 hours. The bacteria which are in the logarithmic phase of growth are centrifuged and washed twice with a buffered physiological saline solution. A cell suspension in saline solution with an extinction of 50 units is then adjusted in a photometer (Dr Lange LP 2W). After a dilution step (1:15), this suspension is mixed 1:1 with a 10% mucine suspension. 0.2 ml of this infection solution is administered i.p. per 20 g of mouse. This corresponds to a cell count of about 1-2×10⁶ microbes/mouse. The i.v. therapy takes place 30 minutes after the infection. Female CFW1 mice are used for the infection experiment. The survival of the animals is recorded for 6 days. The animal model is adjusted so that untreated animals die within 24 h after the infection. It was possible to demonstrate in this model a therapeutic effect of ED₁₀₀=1.25 mg/kg for the compound of Example 2.

Determination of the Spontaneous Resistance Rates to S. aureus

The spontaneous resistance rates for the compounds of the invention are determined as follows: the bacterial microbes are cultivated in 30 ml of a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the addition of 0.4% BH broth) at 37° C. overnight, centrifuged at 6000×g for 10 min and resuspended in 2 ml of a phosphate-buffered physiological NaCl solution (about 2×10⁹ microbes/ml). 100 μl of this cell suspension, and 1:10 and 1:100 dilutions, are plated out on predried agar plates (1.5% agar, 20% defibrinated horse blood, or 1.5% agar, 20% bovine serum in 1/10 Müller-Hinton medium diluted with PBS) which contain the compound of the invention to be tested in a concentration equivalent to 5×MIC or 10×MIC, and incubated at 37° C. for 48 h. The resulting colonies (cfu) are counted.

Isolation of the Biphenomycin-Resistant S. aureus Strains RN4220BiR^{R} and T17

The S. aureus Strain RN4220BiR^{R} is isolated in vitro. For this purpose, 100 μl portions of an S. aureus RN4220 cell suspension (about 1.2×10⁸ cfu/ml) are plated out on an antibiotic-free agar plate (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the addition of 0.4% BH broth and 1% agarose) and on an agar plate containing 2 μg/ml biphenomycin B (10×MIC), and incubated at 37° C. overnight. Whereas about 1×10⁷ cells grow on the antibiotic-free plate, about 100 colonies grow on the antibiotic-containing plate, corresponding to a resistance rate of 1×10⁻⁵. Some of the colonies grown on the antibiotic-containing plate are tested for the biphenomycin B MIC. One colony with an MIC of >50 μM is selected for further use, and the strain is referred to as RN4220Bi^{R}.

The S. aureus Strain T17 is isolated in vivo. CFW1 mice are infected intraperitoneally with 4×10⁷ S. aureus 133 cells per mouse. 0.5 h after the infection, the animals are treated intravenously with 50 mg/kg biphenomycin B. The kidneys are removed from the surviving animals on day 3 after the infection. After homogenization of the organs, the homogenates are plated out as described for RN4220BiR^{R} on antibiotic-free and antibiotic-containing agar plates and incubated at 37° C. overnight. About half the colonies isolated from the kidney show growth on the antibiotic-containing plates (2.2×10⁶ colonies), demonstrating the accumulation of biphenomycin B-resistant S. aureus cells in the kidney of the treated animals. About 20 of these colonies are tested for the biphenomycin B MIC, and a colony with a MIC of >50 μM is selected for further cultivation, and the strain is referred to as T17.

### B. Exemplary Embodiments of Pharmaceutical Compositions

The compounds of the invention can be converted into pharmaceutical preparations in the following way:

Solution which can be Administered Intravenously:

Composition:

1 mg of the compound of Example 1, 15 g of polyethylene glycol 400 and 250 g of water for injections.

Preparation:

The compound of the invention is dissolved together with polyethylene glycol 400 in the water with stirring. The solution is sterilized by filtration (pore diameter 0.22 μm) and dispensed under aseptic conditions into heat-sterilized infusion bottles. These are closed with infusion stoppers and crimped caps.

What is claimed is:

1. A compound of formula 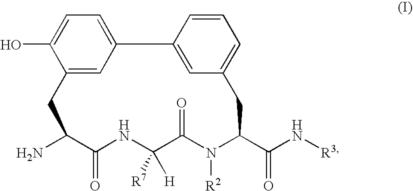
in which
R²⁶ represents hydrogen, halogen, amino or methyl,
R⁷ represents a group of formula 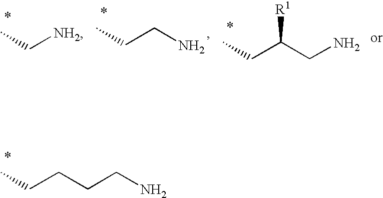
whereby
R¹ represents hydrogen or hydroxy,
* is the linkage site to the carbon atom,
R² represents hydrogen or methyl,
R³ represents a group of formula 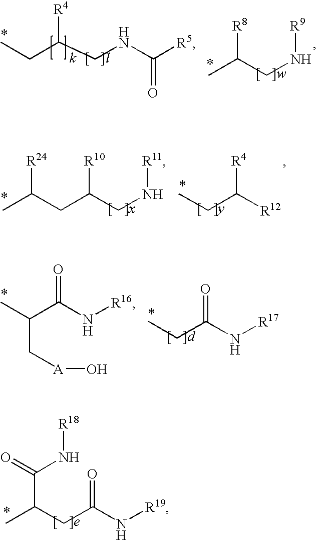
whereby
* is the linkage site to the nitrogen atom,
A represents a bond or phenyl,
R⁴ represents hydrogen, amino or hydroxy,
R⁵ represents a group of formula 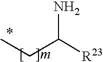
wherein
* is the linkage site to the carbon atom,
R²³ represents hydrogen or a group of formula *—(CH₂)ₙ—OH or *—(CH₂)ₒ—NH₂,
wherein
* is the linkage site to the carbon atom,
n and o independently of one another are a number 1, 2, 3 or 4,
m is a number 0 or 1,
R⁸ and R¹² independently of one another represent a group of formula * CONHR¹⁴ or *—CH₂CONHR¹⁵,
wherein
* is the linkage site to the carbon atom,
R¹⁴ and R¹⁵ independently of one another represent a group of formula 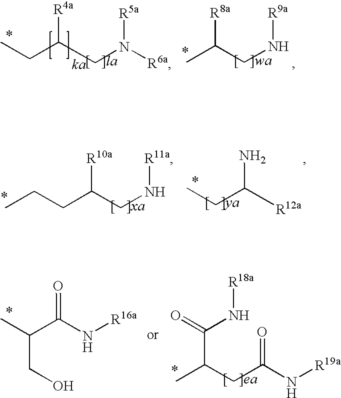
wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
wherein
* is the linkage site to the carbon atom,
Z1a and Z2a independently of one another are a number 1, 2 or 3,
R^{13a} represents hydrogen or methyl,
and
R^{14a} and R^{15a} independently of one another represent a group of formula 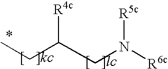
wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula 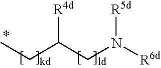
wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
R^{18a} and R^{19a} independently of one another represent hydrogen or a group of formula 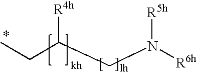
wherein
* is the linkage site to the nitrogen atom,
R^{4h} represents hydrogen, amino or hydroxy,
R^{5h} represents hydrogen, methyl or aminoethyl,
R^{6h} represents hydrogen or aminoethyl,
or
R^{5h} and R^{6h} together with the nitrogen atom to which they are bonded form a piperazine ring,
kh is a number 0 or 1
and
lh is a number 1, 2, 3 or 4,
whereby R^{18a} and R^{19a} are not simultaneously hydrogen,
ka is a number 0 or 1,
ea is a number 1, 2 or 3,
and
la, wa, xa and ya independently of one another are a number 1, 2, 3 or 4,
R⁹ and R¹¹ independently of one another represent hydrogen, methyl, *—C(NH₂)═NH or a group of formula 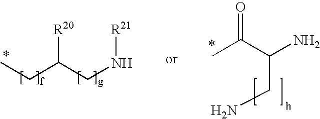
wherein
* is the linkage site to the nitrogen atom,
R²⁰ represents hydrogen or *—(CH₂)ᵢ—NHR²²,
wherein
R²² represents hydrogen or methyl,
and
i is a number 1, 2 or 3,
R²¹ represents hydrogen or methyl,
f is a number 0, 1, 2 or 3,
g is a number 1, 2 or 3,
and
h is a number 1, 2, 3 or 4,
or
R⁸ represents *—(CH₂)_{Z1}—OH,
wherein
* is the linkage site to the carbon atom,
Z1 is a number 1, 2 or 3,
and
R⁹ represents a group of formula 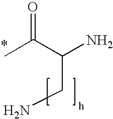
wherein
* is the linkage site to the nitrogen atom, and
h is a number 1, 2, 3 or 4,
R¹⁰ represents amino or hydroxy,
R¹⁶ and R¹⁷ independently of one another represent a group of formula 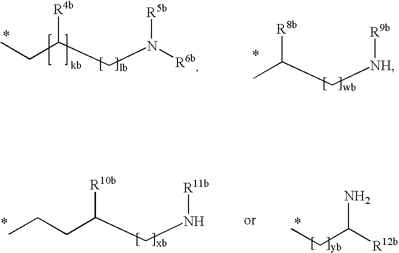
wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *—(CH₂)_{Z1b}—OH, *—(CH₂)_{Z2b}—NHR^{13b}, *—CONHR^{14b} or *—CH₂CONHR^{15b},
wherein
* is the linkage site to the carbon atom,
R^{13b} represents hydrogen or methyl,
and
Z1b and Z2b independently of one another are a number 1, 2 or 3,
and
R^{14b} and R^{15b} independently of one another represent a group of formula 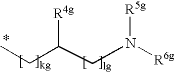
wherein
* is the linkage site to the nitrogen atom,
R^{4g} represents hydrogen, amino or hydroxy,
R^{5g} represents hydrogen, methyl or aminoethyl,
R^{6g} represents hydrogen or aminoethyl,
kg is a number 0 or 1,
and
lg is a number 1, 2, 3 or 4,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
R¹⁸ and R¹⁹ independently of one another represent hydrogen or a group of formula 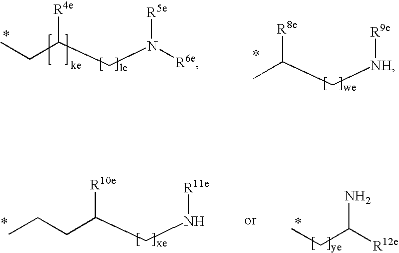
wherein
* is the linkage site to the nitrogen atom,
R^{4e} represents hydrogen, amino or hydroxy,
R^{5e} represents hydrogen, methyl or aminoethyl,
R^{6e} represents hydrogen or aminoethyl,
or
R^{5e} and R^{6e} together with the nitrogen atom to which they are bonded form a piperazine ring,
R^{8e} and R^{12e} independently of one another represent *—(CH₂)_{Z1e}—OH or *—(CH₂)_{Z2e}—NHR^{13e},
wherein
* is the linkage site to the carbon atom,
R^{13e} represents hydrogen or methyl,
and
Z1e and Z2e independently of one another are a number 1, 2 or 3,
R^{9e} and R^{11e} independently of one another represent hydrogen or methyl,
R^{10e} represents amino or hydroxy,
ke is a number 0 or 1, and
le, we, xe and ye independently of one another are a number 1, 2, 3 or 4,
whereby R¹⁸ and R¹⁹ are not simultaneously hydrogen,
R²⁴ represents a group of formula *—CONHR²⁵,
wherein
* is the linkage site to the carbon atom,
R²⁵ represents a group of formula 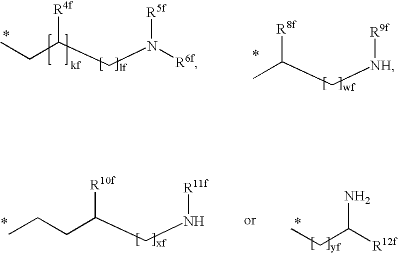
wherein
the linkage site to the nitrogen atom,
R^{4f} represents hydrogen, amino or hydroxy,
R^{5f} represents hydrogen, methyl or aminoethyl,
R^{6f} represents hydrogen or aminoethyl,
or
R^{5f} and R^{6f} together with the nitrogen atom to which they are bonded form a piperazine ring,
R^{8f} and R^{12f} independently of one another represent *—(CH₂)_{Z1f}—OH or *—(CH₂)_{Z2f}—NHR^{13f},
wherein
* is the linkage site to the carbon atom,
R^{13f} represents hydrogen or methyl, and
Z1f and Z2f independently of one another are a number 1, 2 or 3,
R^{9f} and R^{11f} independently of one another represent hydrogen or methyl,
R^{10f} represents amino or hydroxy,
kf is a number 0 or 1,
and
lf, wf, xf yf independently of one another are and a number 1, 2, 3 or 4,
d and e independently of one another are a number 1, 2 or 3,
k is a number 0 or 1,
l, w, x and y independently of one another are a number 1, 2, 3 or 4, 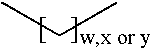
independently of one another may when w, x or y equals 3 carry a hydroxy group,
or one of its salts, its solvates or the solvates of its salts.

2. The compound of claim 1, corresponding to formula 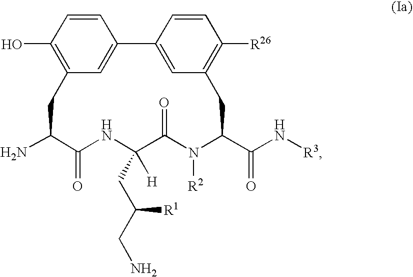
in which
R²⁶ represents hydrogen, halogen, amino or methyl,
R¹ represents hydrogen or hydroxy,
R² represents hydrogen or methyl,
R³ is as defined in claim 1,
or one of its salts, its solvates or the solvates of its salts.

3. The compound of claim 1, wherein
R²⁶ represents hydrogen, chlorine or methyl.

4. The compound of claim 1, wherein
R³ represents a group of formula 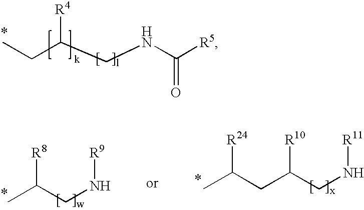
whereby
* is the linkage site to the nitrogen atom,
R⁴ represents hydrogen, amino or hydroxy,
R⁵ represents a group of formula 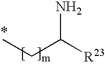
wherein
* is the linkage site to the carbon atom,
R²³ represents hydrogen or a group of formula *—(CH₂)ₙ—OH or *—(CH₂)ₒ—NH₂,
wherein
* is the linkage site to the carbon atom,
n and o independently of one another are a number 1, 2, 3 or 4,
m is a number 0 or 1,
R⁸ represents a group of formula *—CONHR¹⁴ or *—CH₂CONHR¹⁵,
wherein
* is the linkage site to the carbon atom,
R¹⁴ and R¹⁵ independently of one another represent a group of formula 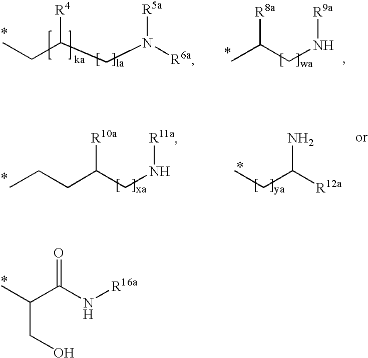
wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{14a} or *—CH₂CONHR^{15a},
wherein
* is the linkage site to the carbon atom,
Z1a and Z2a independently of one another are a number 1, 2 or 3,
R^{13a} represents hydrogen or methyl,
and
R^{14a} and R^{15a} independently of one another represent a group of formula 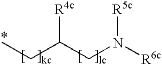
wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula 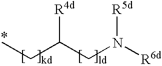
wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
R⁹ and R¹¹ independently of one another represent hydrogen, methyl, *—C(NH₂)═NH or a group of formula 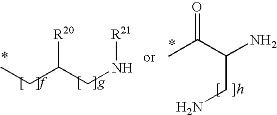
wherein
* is the linkage site to the nitrogen atom,
R²⁰ represents hydrogen or *—(CH₂)ᵢ—NHR²²,
wherein
R²² represents hydrogen or methyl, and
i is a number 1, 2 or 3,
R²¹ represents hydrogen or methyl,
f is a number 0, 1, 2 or 3,
g is a number 1, 2 or 3,
and
h is a number 1, 2, 3 or 4,
or
R⁸ represents *—(CH₂)_{Z1}—OH
wherein
* is the linkage site to the carbon atom,
Z1 is a number 1, 2 or 3,
and
R⁹ represents a group of formula 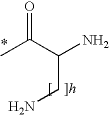
wherein
* is the linkage site to the nitrogen atom,
and
h is a number 1, 2, 3 or 4,
R¹⁰ represents amino or hydroxy,
R²⁴ represents a group of formula *—CONHR
wherein
* is the linkage site to the carbon atom,
R²⁵ represents a group of formula 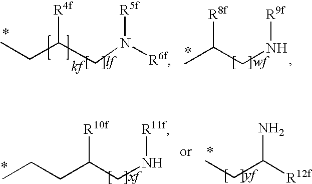
wherein
* is the linkage site to the nitrogen atom,
R^{4f} represents hydrogen, amino or hydroxy,
R^{5f} represents hydrogen, methyl or aminoethyl,
R^{6f} represents hydrogen or aminoethyl,
or
R^{5f} and R^{6f} together with the nitrogen atom to which they are bonded form a piperazine ring,
R^{8f} and R^{12f} independently of one another represent *—(CH₂)_{Z1f}—OH or *—(CH₂)_{Z2f}—NHR^{13f},
wherein
* is the linkage site to the carbon atom,
R^{13f} represents hydrogen or methyl, and
Z1f and Z2f independently of one another are a number 1, 2 or 3,
R^{9f} and R^{11f} independently of one another represent hydrogen or methyl,
R^{10f} represents amino or hydroxy,
kf is a number 0 or 1,
and
lf, wf, xf and yf independently of one another are a number 1, 2, 3 or 4,
k is a number 0 or 1,
l, w and x independently of one another are a number 1, 2, 3 or 4, 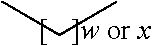
independently of one another may when w or x equals 3 carry a hydroxy group,
or one of its salts, its solvates or the solvates of its salts.

5. The compound of claim 1, wherein
R³ represents a group of formula 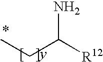
whereby
* is the linkage site to the nitrogen atom,
R¹² represents a group of formula *—CONHR¹⁴ or *—CH₂CONHR¹⁵,
wherein
* is the linkage site to the carbon atom,
R¹⁴ and R¹⁵ independently of one another represent a group of formula 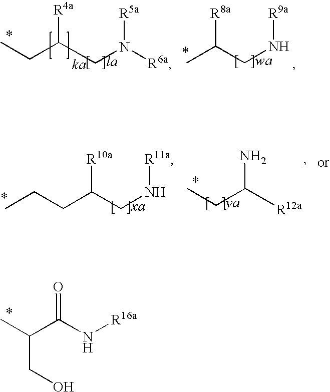
wherein
* is the linkage site to the nitrogen atom,
R^{4a} represents hydrogen, amino or hydroxy,
R^{5a} represents hydrogen, methyl or aminoethyl,
R^{6a} represents hydrogen or aminoethyl,
or
R^{5a} and R^{6a} together with the nitrogen atom to which they are bonded form a piperazine ring,
R^{8a} and R^{12a} independently of one another represent *—(CH₂)_{Z1a}—OH, *—(CH₂)_{Z2a}—NHR^{13a}, *—CONHR^{4a} or *—CH₂CONHR^{15a},
wherein
* is the linkage site to the carbon atom,
Z1a and Z2a independently of one another are a number 1, 2 or 3,
R^{3a} represents hydrogen or methyl,
and
R^{14a} and R^{15a} independently of one another represent a group of formula 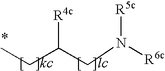
wherein
* is the linkage site to the nitrogen atom,
R^{4c} represents hydrogen, amino or hydroxy,
R^{5c} represents hydrogen, methyl or aminoethyl,
R^{6c} represents hydrogen or aminoethyl,
kc is a number 0 or 1,
and
lc is a number 1, 2, 3 or 4,
R^{9a} and R^{11a} independently of one another represent hydrogen or methyl,
R^{10a} represents amino or hydroxy,
R^{16a} represents a group of formula 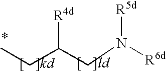
wherein
* is the linkage site to the nitrogen atom,
R^{4d} represents hydrogen, amino or hydroxy,
R^{5d} represents hydrogen, methyl or aminoethyl,
R^{6d} represents hydrogen or aminoethyl,
kd is a number 0 or 1,
and
ld is a number 1, 2, 3 or 4,
ka is a number 0 or 1,
and
la, wa, xa and ya independently of one another are a number 1, 2, 3 or 4,
y is a number 1, 2, 3 or 4, 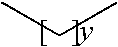
may when y equals 3 carry a hydroxy group,
or one of its salts, its solvates or the solvates of its salts.

6. The compound of claim 1, wherein
R³ represents a group of formula 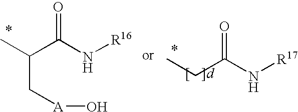
whereby
* is the linkage site to the nitrogen atom,
A represents a bond or phenyl,
R¹⁶ and R¹⁷ independently of one another represent a group of formula 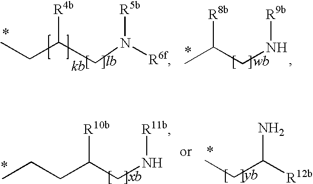
wherein
* is the linkage site to the nitrogen atom,
R^{4b} represents hydrogen, amino or hydroxy,
R^{5b} represents hydrogen, methyl or aminoethyl,
R^{6b} represents hydrogen or aminoethyl,
or
R^{5b} and R^{6b} together with the nitrogen atom to which they are bonded form a piperazine ring,
R^{8b} and R^{12b} independently of one another represent *—(CH₂)_{Z1b}—OH or *—(CH₂)_{Z2b}—NHR^{13b},
wherein
* is the linkage site to the carbon atom,
R^{13b} represents hydrogen or methyl,
and
Z1b and Z2b independently of one another are a number 1, 2 or 3,
R^{9b} and R^{11b} independently of one another represent hydrogen or methyl,
R^{10b} represents amino or hydroxy,
kb is a number 0 or 1,
lb, wb, xb and yb independently of one another are a number 1, 2, 3 or 4,
d is a number 1, 2 or 3,
or one of its salts, its solvates or the solvates of its salts.

7. The compound of claim 1, wherein
R³ represents a group of formula 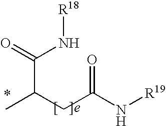
whereby
* is the linkage site to the nitrogen atom,
R¹⁸ and R¹⁹ independently of one another represent hydrogen or a group of formula 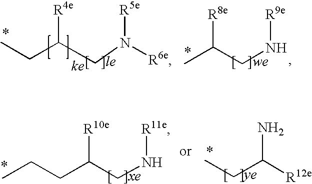
wherein
* is the linkage site to the nitrogen atom,
R^{4e} represents hydrogen, amino or hydroxy,
R^{5e} represents hydrogen, methyl or aminoethyl,
R^{6e} represents hydrogen or aminoethyl,
or
R^{5e} and R^{6e} together with the nitrogen atom to which they are bonded form a piperazine ring,
R^{8e} and R^{12e} independently of one another represent *—(CH₂)_{Z1e}—OH or *—(CH₂)_{Z2e}—NHR^{13e},
wherein
* is the linkage site to the carbon atom,
R^{13e} represents hydrogen or methyl,
and
Z1e and Z2e independently of one another are a number 1, 2 or 3,
R^{9e} and R^{11e} independently of one another represent hydrogen or methyl,
R^{10e} represents amino or hydroxy,
ke is a number 0 or 1,
and
le, we, xe and ye independently of one another are a number 1, 2, 3 or 4,
whereby R¹⁸ and R¹⁹ are not simultaneously hydrogen,
e is a number 1, 2 or 3,
or one of its salts, its solvates or the solvates of its salts.

8. A method for preparing a compound of formula (I) of claim 1 or one of its salts, solvates or solvates of its salts, wherein
[A] a compound of formula 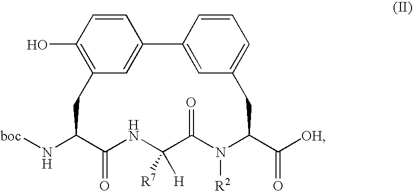
wherein R², R⁷ and R²⁶ have the meaning indicated in claim 1, and boc is tert-butoxycarbonyl,
* is reacted in a two-stage process firstly in the presence of one or more dehydrating reagents with a compound of formula
H₂NR³ (III),
wherein R³ has the meaning indicated in claim 1,
and subsequently with an acid, by hydrogenolysis, or with an acid and by hydrogenolysis
or
[B] a Compound of Formula 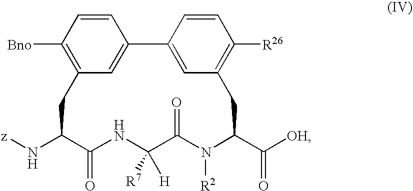
wherein R², R⁷ and R²⁶ have the meaning indicated in claim 1, and Z is benzyloxycarbonyl,
* is reacted in a two-stage process firstly in the presence of one or more dehydrating reagents with a compound of formula
H₂NR³ (III),
wherein R³ has the meaning indicated in claim 1,
and subsequently with an acid or by hydrogenolysis.

9. A method for preparing a compound of formula (I) of claim 1 or one of its solvates, wherein a salt of said compound or a solvate of a salt of said compound is converted into said compound by chromatography with the addition of a base.

10. The compound of claim 1 for the treatment and/or prophylaxis of diseases.

11. A method for the production of a medicament for the treatment, prophylaxis or treatment and prophylaxis of diseases using a compound of claim 1.

12. A method for the production of a medicament for the treatment, prophylaxis or treatment and prophylaxis of bacterial diseases using a compound of claim 1.

13. A medicament comprising at least one compound of claim 1 in combination with at least one inert, non-toxic, pharmaceutically suitable excipient.

14. The medicament of claim 13 for the treatment, prophylaxis or treatment and prophylaxis of bacterial infections.

15. A method for controlling bacterial infections in humans and animals by administering an antibacterially effective amount of at least one compound of claim 1.

16. A method for controlling bacterial infections in humans and animals by administering an antibacterially effective amount of at least one medicament of claim 13.
